SEQUENCE LISTING (115 sequences)

SEQ ID NO: 1 - DNA, 401 bp, Lycopersicon esculentum
  Polymorphism (201)..(201): A=susceptible allele ; G=resistant allele
gttgggaggcagaagggtataagattggacactgaaatttttgctacttaccgtaagctcatcaacatcgactgttagaaacatcagagaaagatatttctcagacagctcacagtagaatggagagatcattctacatggaccacaccatgaagcactgaaatttgcaataacctgccaaaatttcacttgcgtaagaarggtccaaaatgccaaagccagcaaaaatgtaaaattttaagcaagcaaaaggcgagggatccatatggggaaactgctgcaccattctgctattatccttctcttcgcatgtgtgcaaagcattagagttaaagtgccttgagtattgttttttaacgtcttttgaattttagtgcagctgggctagaggattaagttac

SEQ ID NO: 2 - DNA, 401 bp, Lycopersicon esculentum
  Polymorphism (201)..(201): A=susceptible allele ; G=resistant allele
tactgtaccaatattcagtgcaaggtataggtgtccattcatgagggctgtcacccatgaacaccgaaaatgctgctcacagtacttgctgagagcttcagacgaaccatccatttgcagataatatccagtgctcaagataggggtgtttgttggagccggtgaattttttccagaagagagtatatgtttgggatcacrgtctgtgacaatgacctcgggatgtccatgtagcctaataatttcagaagcaaatgaagaagccatagtttgagctgtgaaggtagaagaaaaggcaatgattgattgcccatatttgagttaccagtcaaccaacgtcaagattgttaccttcccttcgaccccaaaaggcaagtaatgcattggtacctccttaaaca

SEQ ID NO: 3 - DNA, 401 bp, Lycopersicon esculentum
  Polymorphism (201)..(201): G=susceptible allele ; A=resistant allele
tactatcgaagggtaaaaataaaaaacaagttgttcaaatatccattagaatgaagaaaagaaaagcaagttaggggctcttggaccgggtggtatcaatgtctaagcaatggttctccagaggcgtcacaccattccaagccttattcagcatggagaaaaatccctgaaatctaatggctcaagtctagaaatagcctrgtcgatagacacacaagtaaaaattacctttaccgtcttctacatcattagcattaggcgttctctgaccactcccttcctcttctccttttgcaaatgacgacagaacatctcgacttcgagggaagttttgggtgggattggagtaaaatcgtgtattcactccccatactcagcctccaagtcctatttaacctgtg

SEQ ID NO: 4 - DNA, 401 bp, Lycopersicon esculentum
  Polymorphism (201)..(201): G=susceptible allele ; A=resistant allele
aagtaggaatacggtccatggtctgtgttcatggattgagacataacttacccaatcacttaaatgaacaacagatgaccagcgcggagcattgttcaatctattgtccgtatgtttgatcataactgaggttagcagttagttacgggaaatttacggattggatcaacttaaaattgtcataaattttagcacaaaatraattaggttctccataacccatgatatgatagataatttaatattctctccatatccactgagtttgctaaaatttgacctccgagtgaaaagttatgcctattttagtgaaggcctattgagtagaccctaacgacagaccgtctattgaacaacggcccatcagtccaggtcgtcgatttccgcgacaacaattagct

SEQ ID NO: 5 - DNA, 401 bp, Lycopersicon esculentum
  Polymorphism (201)..(201): C=susceptible allele ; A=resistant allele
aacaacccaatattcttaggaaatgatccttaaaatggtgcatcttgcttagacaaccaatgtaagggctacttgactagagagagaagtctctgggatgattgaagctaccacaacatttggtttagctcccctccatgtgtctattgatgatttgaccgctagggtcacaacttgtgagagcaagaaaggggagaaatmtgaggctttgtcttttaaagacaaggtagcaaagcagaagaaggacatagactatccaaagtctactgacttcacttcattactagaggctgctaatgatttagaaacaattgatactttagagattacttcgcctaccaccagagaggtacatagggaggatgctagaattgatgtattagatgctgaaactgatgagg

SEQ ID NO: 6 - DNA, 401 bp, Lycopersicon esculentum
  Polymorphism (201)..(201): G=susceptible allele ; A=resistant allele
cattgcgaaattgcatttgttctcttaatcaaagtcttgtaactttgggtgatttagtgtgttctcggtgaaccgatcggcaactcaccgactacacatttatgtcgccgacttgatttttccccctcccctccgggctagtatacttgaactgtaggcgaattggggagccactcggcagttcaccaattggttttggcrattaccaggatcttctttttctccacttgttcagctcgtttttctcccttttttctagaagtgtcctgccttgcttattaactcatataccttaaaattaatgctttaacaatagtttgtagaataaaataggcatttaaggacactcaaactaccaaaaaatatccctaaatgagtaaaatctttgactcattatttat

SEQ ID NO: 7 - DNA, 401 bp, Lycopersicon esculentum
  Polymorphism (201)..(201): G=susceptible allele ; A=resistant allele
gtaatagggtcataaaatacttggtgtgaatttaccaaacaccctaagcattggcagaaaacatatagtgtttgaacccctcaaagacgagacctaacaaatagaccatctgcgcatcaacggaccgtttgtggggtctcgtgggttgacacttagctttacatggagcctgactaacaaaccttgaagatccaacagatrgagcaagaagacaggccatcggttcaaccattggctgtcgttgcctttgttggtgaacacctcaaggaaatttttaaccaagatttgggctctttctctagggccccttgtgggtcaaaggtggggtcgtactaaacaattaaattccttaactatgtaatataggtttaggaatatctcatgcaggttccaataaaaaa

SEQ ID NO: 8 - DNA, 401 bp, Lycopersicon esculentum
  Polymorphism (201)..(201): A=susceptible allele ; C=resistant allele
tgaaatgtttaaggtttagattggttggttcgctcacataggagggtaagtgtgggtgccagccccagcccggttttgggtcgtgacaattggacttccatctgtggtgattttaatacaccgatatgggtttttcccatgctattatcttcactttagtttcatgagtgcaagcatcccctagttgaaagagacctttcmacattgaaggccatttgatttaaggaaatgttacagccaacattctgtatgtatccataataataacatattttactctactcatgtcggatgctcttccttaatatatgcttgcgcacctattctttcacaagatccaacatattatgacatgtctggcttataagagtagtttgtgtgccccattttggtgttttgtt

SEQ ID NO: 9 - DNA, 401 bp, Lycopersicon esculentum
  Polymorphism (201)..(201): G=susceptible allele ; C=resistant allele
gacttgactcttaacttttagtcaattgaattatggattcaaagttaattatctcatgtttatggatgattttaagatttttgagtgtaccttaaagtgtgggaataaactagaaaacataggtatgttgctagggtactagtacgaaatgaagtgggaaaagattgatgaattggtaaccctggcgcattaagaggtgcsggccgcaagtgggtaacatcagatactagtgtagggttttttggctggtatggagccccagattccaaacttcagcgcccctaatgaggcgctttggctggaacagcgccttagtcccttgcccagttgaattccaacttttcttgctcatttttcaactctaaacccctaatttcaacttgattctttacccaaacaca

SEQ ID NO: 10 - DNA, 401 bp, Lycopersicon esculentum
  Polymorphism (201)..(201): G=susceptible allele ; A=resistant allele
tatgagagagaattttttaaaccatctaatccatgaaaaggcttcacagacattagatcagtacaaggaccgtcaatggggttcgtcaacctaaaactacagacagtgtgatatttttatttttttccattgttagctcgtcatcctaaaagtgttttgagtgtgccgttaagatatattggtactaaaaatttctccgcrggtactatgtctcccaagaggcaagtggtttatacaaaaaggggcaaattcaagttagttggcccttcttcttggttaattgatgaagacctagactagaagagggacccagcctacattccttcggacataaaaacaccaccaacaaaaccctaaactactaagagcaccattctaatgaggaccgcacactgttcggc

SEQ ID NO: 11 - DNA, 401 bp, Lycopersicon esculentum
  Polymorphism (201)..(201): A=susceptible allele ; C=resistant allele
gaactatctaacttctttcatcctccttttatgcgatagagttcatctttataaaagtttctttctaactcgtgcttgcacgtatcttttagatcatgcctccatgaagatttgtctgagctagtcgttctaggagcaacgttaaggatcaagaggtgcccaatgcactagaagtgcgaccctaaggagaggtcacttctmggatcggataatgagacagattgcgaccaacaaagctatgaaataaagaaagaattgacaggagctagttgatacttctagcattcgtgagtacttaaggatgaatttttcaagcttcaataattcaagtgtgactgaggatccagaatattttataaaatagttgtagaaagtgattgaggtcatgcgtgttgcttatg

SEQ ID NO: 12 - DNA, 401 bp, Lycopersicon esculentum
  Polymorphism (201)..(201): A=susceptible allele ; C=resistant allele
aaaatttaaccatcaaatcttcattaaacatagacgtatgggatctaattcacctttgagacctctttcctcaaaggttttcttcaaagtattttcaaaagttgagttttcttgtaggtgcattcatccgaatttggacctcccacagtggttcaaagttcaaaaatttgatgtcattggaggaatagggaaccccttagmgcatctaaggtcctattgttacaaactaatgggagttaggagtgataaagctatattgatgcaactttttggcagaagattgagcggagaagctttggaactgtttaaatctcacgagaccagacaatgagattgctggaatgcctcggctaatgaattcattgagcgagttgcttacaacgtagagatcgtccctgact

SEQ ID NO: 13 - DNA, 401 bp, Lycopersicon esculentum
  Polymorphism (201)..(201): A=susceptible allele ; G=resistant allele
agttagtgagttctatagtctttggcaagagttttggagtataatatgatggtaaaagcttgtaaaaatggtttccgtacttacaataggagtttaaggggtccgggtacgactccccaaataccaccaaggggtccttgaggaggaccctaacactgaccaaagagatttcaaaacaacttggttgatggttgcaattcrtgaccagtaggttggtctacaccccgtaagtcaagtcatgcaccactcctacaaagctgatcctcgggtgcaaaccacaaaggtggttgacgtacagtaacttctcctacagtccgtaggtggaggggtgtaggtgtttccagttttaagtaatttattttagttaggtgtttggtaatcgattagttatacaattacgg

SEQ ID NO: 14 - DNA, 401 bp, Lycopersicon esculentum
  Polymorphism (201)..(201): A=susceptible allele ; G=resistant allele
cattaagagactaaggtcgtggggtaacaatccatggaccaggaccacgacctgtgacctggcctaagatccgtggatctggcagtgggtcgacccttagaggattttctggacctttttgggaagggttgtaggttgaaatccacggaccaccacttacagtctgtgcgtcaccctagtggtaactatggattgcacatrtagcttctggaattaacatgcaacttttggatgttggttggtctttttaggataaaaggtgttacattatctcccccttggaaataatcttcctcgagtgaagactaaactagctgaatatgaaggaaaagagcttccaaccctaccactaattactagaaactgatttctgactgaaataagttccaaggacatgcaaa

SEQ ID NO: 15 - DNA, 401 bp, Lycopersicon esculentum
  Polymorphism (201)..(201): C=susceptible allele ; A=resistant allele
gaactatgagcttctgtcaacatctaatttgtcaactcatcagcatttaaaacacataattgactttgatatcgaggacaccatctccccattgtgcaaaagcctcaatgaaattttcagatgttgacttctataagtcaactcataccagatcaagatcttgcttacccttcacatctgatacaaaagatgattccaaamaattttgaacctctacacaaccttcattagaatataccatctaaacatccaatctggaaaatctatgaaaatcatgaacagaatctttcttatcatcctcaacaggagatacactacctaactataaccgactaagaacatttgtaaccaaatttatatttttaggatgatatagggaacttatgtcataatccttctac

SEQ ID NO: 16 - DNA, 401 bp, Lycopersicon esculentum
  Polymorphism (201)..(201): G=susceptible allele ; A=resistant allele
gtcattattataattagaatcaataggaagtgataaaacacttcagaaagacttaaacggacagaatagcgaaaagctggtaaaaatgatgatttgggtgcatcttgggcgcaccccaccagctccgaattgattagggctcaaaaatagcgcactgctagcatgctactctagtctgcgtggcgctatttgggactgtcrtgccatcctttacttgggaaaagattggcaaacttttgatctcattttttaagttttaaatcgattaatttcgattacttttctaaaattcactttaatgtaagatatccaaagcatatacacaaaatctaactcaaataactcaaggaaacaacactttaatatataaaaaaaactctttaatctcaacctaactcaag

SEQ ID NO: 17 - DNA, 401 bp, Lycopersicon esculentum
  Polymorphism (201)..(201): G=susceptible allele ; A=resistant allele
ttagctagtggatccacctaaaagatgaaatatataggtttaccttaggaaaataggacatatgtttttgtgtgggacatgacaccagattctatacatctcacattatttatgtcggttaaggctaattccctacatgacaagatcatgaacataaactatatttatgacttctcaaggggttctacttagtgtgagtargggtatgagacttcactcctgtattccacgagtagctttgaaaaggttttatagtgtggttttcttaatctatgaatatatatgaatttatgtatgtacaatatggattattgctataagtggatttcgttgttacaagttgattaacatatatgtttcattgtttatggatgaggtcaaggtatgttatgtatgattag

SEQ ID NO: 18 - DNA, 401 bp, Lycopersicon esculentum
  Polymorphism (201)..(201): A=susceptible allele ; G=resistant allele
gcagcatgtctcattaggtctatctctcagttagtagctagccttttaaaataatacttcaacctagacagacaaactagccaataatatttatttatgttagaaacaaccaacaaactcactatttttttaatttataattcttgaaaattaccttcttgtcgatttctttatccaaaagaagaagaaagaaaaaataaratagtctcattgtcacttacactcacatcattagtggaatatcttttgcataaaaaccacaaacacatataaagggtgattccaattaaacttccctataattgacaattacctattcgttaacagaatctagagatattatgacaatgatattacacatacatacactgaatattaaaagtctatatgattaacagaaa

SEQ ID NO: 19 - DNA, 401 bp, Lycopersicon esculentum
  Polymorphism (201)..(201): C=susceptible allele ; A=resistant allele
tcatgaggcttctctcgggtagcggcataaggaaatcatacagagggtgtagtatcatcattgtgactggccattttgagagagattacaaaaaataaggtaagttaggatagtatagacacgatagggatgcctagacggaaagagaatttttttgggaaaattaagccaatctactgacctaagtcatcttgatgtagmggcaatcatcccgctacaacgggatgatgcaggccactacaacgaccttaatctaggacgaacttcaaacgcaaagagggcgtatttttgtcccaaaatctgagttttatcagttttttagcatatcaaagtctataaactatcctagaacaagaactaactaacatgtcaaccaaataactcctaaattgtgtctaaat

SEQ ID NO: 20 - DNA, 401 bp, Lycopersicon esculentum
  Polymorphism (201)..(201): G=susceptible allele ; A=resistant allele
ttgattactaacaacaagtgacttgattaataagacaacaattgtttcatgatagaagattgaatcactagtgaagtcttctcgaagtataacttcaatttttaagaactccagttatgaacttcatgataattctgagttcaagattcctcatcatcttttgtcttgggggcaacatttaaatcaaacttcagatgctcraccaaaatagattttgtgtttctttttttactttctccatccttaacttcaatgtcaacttattctccttcatatgcttgtcttgaggtaattctgagcaaaggtgcagaatctcatgcattagcactctctttttttttttccgttacatgtttgcaattctttatagagatcttccaatgcctcaaaattatttgatt

SEQ ID NO: 21 - DNA, 401 bp, Lycopersicon esculentum
  Polymorphism (201)..(201): A=susceptible allele ; C=resistant allele
gttttcttattgtctattccaatacattacttctaacttttctatttccagatgaagatacattcatattaaagagatttcaccatctcacaaagagctagcctagatgtaaaatgttaatgcactaatcttatcgaagatgaaaacataaatggaaattaatgaatgtatgagcgttttctttgatttatcacttttccmatattcactagttattagaaagttcaactaaatttatcaatttaggcaatataaggcctcattaaagtctgaggatgaaaaaagctgacttaataacataagaaatccataatctttcatttttaatctaaaacctcatatcgatggataattgattactaacaacaagtgacttgattaataagacaacaattgtttca

SEQ ID NO: 22 - DNA, 401 bp, Lycopersicon esculentum
  Polymorphism (201)..(201): G=susceptible allele ; A=resistant allele
taaagtaaagttaggcggcagaagataacacatggaagggacaaggtctttatctgaaggataagtaagtgactaggagactttaattgatgcgaaaaaactcaaatatgccatcgaaattttagaaaagacttatttatatcatcttctagatatttggttaatttatgtcattgttgttaaagaaaagacgcattcacrttttactccaattttacaaaaccattttctacacctcaaataaggtttgacgcttttcaattggagtttttggatcatatgcacactttttgcttctttttctttaagaacaacaagaacacatgaagaacataaacaactcccaaaattttcaacatcttcaactcttcacgaaatcatctcaattttcggctacatca

SEQ ID NO: 23 - DNA, 401 bp, Lycopersicon esculentum
  Polymorphism (201)..(201): T=susceptible allele ; A=resistant allele
acatttgataaatgcaacataaaaaataaattcaatccattcacagaaccaacacatacaaagcaatcaaacttttatcagatgtgatattcgatccaatcaaaagataacccatatcgatgtggtattcgagataatcgatatgtatatcaagggtggtatccgagcctatgaaaagatatcatgtatcaattgtggtawttagaccaattgaatataccacatcacacaatcagaatcataataaatacaatcacatttgtataaacaagtcaattggaaaagtttatcgcatgagattgacgacaaatatcatttcaatttcatccattttctagtccctaaccacatattgtacaagtctactagcaaaataattaacttgaacatattacacaatg

SEQ ID NO: 24 - DNA, 401 bp, Lycopersicon esculentum
  Polymorphism (201)..(201): A=susceptible allele ; G=resistant allele
tatacatgagaaaattgttcgagaagggtcgtcgaggtaggtgatggttgaattatttttcattgtgtaatatgttcaagttaattattttgctagtagacttgtacaatatgtggttagggactagaaaatggatgaaattgaaatgatatttgtcgtcaatctcatgcgataaacttttccaattgacttgtttatacraatgtgattgtatttattatgattctgattgtgtgatgtggtatattcaattggtctaaataccacaattgatacatgatatcttttcataggctcggataccacccttgatatacatatcgattatctcgaataccacatcgatatgggttatcttttgattggatcgaatatcacatctgataaaagtttgattgctt

SEQ ID NO: 25 - DNA, 401 bp, Lycopersicon esculentum
  Polymorphism (201)..(201): G=susceptible allele ; A=resistant allele
atagagccttaattttggtcatgccatctgtggatagtggaactgcacattttgaaggctagagttaagattcaccgcaatagtagaacttgttttagatgatgagttacatttacaccatcatatgcacttactagtagtagatctaaccgggggattcacaaatcccatgatgattggtttcagagactaacgaggtaragtgatgtgatgaataactaacctttgagttttgattggttaaaattagtgtatacttgagtgattccataattatgccatctcctaaaaacaacaagaaggtacgcattaaaactattttagcattcgaggtcaagaatcaaagatctcactttgattataaatagaattaaagatctcgccttcactgtagcttgaat

SEQ ID NO: 26 - DNA, 401 bp, Lycopersicon esculentum
  Polymorphism (201)..(201): G=susceptible allele ; A=resistant allele
ttggtgagtctattctagctgagattgtatcgtgattgtaccatttccgtcaatcacaaaagaaccatggatgacttagttgagttagatataattttgaattcattctaggtatgaatggtttcattcttgttatgactcaatcgattgaagaactcgagttgtgaagttcaaaatccctaatgagacacttattgagtrgagaagtagttttgtagtgcctaagggtcatttttttttgtatcttaaggcgataaagttagtttctaagggttgttcctatcactttgttagagttaacgactctagtgttgagatgcctcttattcaacaagtcgcagttgtgagtgaagtttccagatgatcttcccgaagtccctcatatgagaaatagacctcga

SEQ ID NO: 27 - DNA, 401 bp, Lycopersicon esculentum
  Polymorphism (201)..(201): A=susceptible allele ; G=resistant allele
ctttaaatatgtttttcgaactaaaaaaggataagaaatactccaactagcacaggtattttgtacagaaaataaagaagagccaagtaactttttttaaaaaaaatccaaaagaaactctcatgtttgagcaacatctaatacttctaggacaacaacaaaaaatgcttaaaagatggtaataacactatgaatcatcarattaaactaattgcatggtaacataatgaacagaacacgtatacacctaaagacatggatcatatgcttggactaacgagaatacaataaaataaaataaaacacattgtgtagtgaatggggataatcttaaataaagaaccaacagattgattaattttgaggttctgcctcatactcttgttgcttgaatattggca

SEQ ID NO: 28 - DNA, 401 bp, Lycopersicon esculentum
  Polymorphism (201)..(201): C=susceptible allele ; A=resistant allele
tttgtaaacgaggatatgttttcaacaatattaacaagatcaaatcacattatatttactctcaatacctaacattgatgactacaagacttattctcaaagattcactccttactatcaccattatacttaaggttcagggtattaaaatcacaattttcattagtagatgattctaggcataattcgccatagaggttmaatttcaattttactattcatgatccatattatgcactaaaatctagttcaattcatttctatatgttcctaatcttgaaaattatgaaagttgatatcaaatttagaagatcacccttgaatcctcaattttacctctcatggcataaacccaattaatatcaattctatcaactagactagggttaatcatgaaaatc

SEQ ID NO: 29 - DNA, 401 bp, Lycopersicon esculentum
  Polymorphism (201)..(201): G=susceptible allele ; A=resistant allele
ttaattctgcactagttttcacgtcttatgtaatgaatgatagcgaaaaggctagtacaagaccttcgaaaggttaattacacaattgttccgatccaagggtgccttattctacggtgtactacgggtcatgaaaaattttacccatagagtccattcagaacttgatttctaagcttgcgaatttccccaaaaattagrgttcaatctaagtcaaaatttctctttcaagacctttctaatatttaatttatcaataattatgattgaattgttgattaaatattgatttatgaatccccatatttctaaattgtgaatttatgatttgggttgatcgaacataccaaaatgtgaagttattatgtctatattgattgagttattaactaatgatatta

SEQ ID NO: 30 - DNA, 401 bp, Lycopersicon esculentum
  Polymorphism (201)..(201): C=susceptible allele ; G=resistant allele
cgaggagttcttggagatgagaagttatgaataaatgaatgttatacgcaaaagcctacaatttgggcattttcaaactctacaccacgatctgattactagcaattttgcagtttagcttcaactgaatcccttttgtaaaagtcatcaatactctacttgtttaatcttttcttcataaggacatatttcaggtgaatstagagaaatcgtcgacaatcacgaagatgtatcttttcccacttctgctttaaattctcatttgtccgcaaatatccatgtggagaagctctaatggattgattgcggtgacacctttctttgacctgaaagatgatcttctttgattccttcttacgcaagcctcacaaattttattttctagagaacatttttattcc

SEQ ID NO: 31 - DNA, 401 bp, Lycopersicon esculentum
  Polymorphism (201)..(201): C=susceptible allele ; A=resistant allele
ttaccattgcatgtactataatccatggatttttggtgatacgtcaatccaacttgttttttgacgaatatttttatgggaattggttaggaccttattaattacgtgattggtaagtaggggcaaacaaacactttttaaagttcgaaatagccccaatgtggaagtgaccgaaattactattttacgcctgttatagtmcatgaatttttggtgatatattaatccgtcttgtttttggtcgaaatttttatggacgtccgttaaaaacttattaatggcatgaattggtcaaaagggacaaagtaacaaatgcattttcaagttcaaaatagtccaatgcagtaagagcatccccaagagtttgttctactaactaaaaaacatgttgaggattcaaa

SEQ ID NO: 32 - DNA, 401 bp, Lycopersicon esculentum
  Polymorphism (201)..(201): G=susceptible allele ; C=resistant allele
atgcacaaacaaatttggattatttatgccacctactagaaatatttcactgacttaaagacttgatcctacaataatgtgagcatatatttcttgagtgacatccaaaataggcaataatgcttgtagatatttcattgctagttgtcttctttctgctgtttgattgcatcaacagaaccattactgaaacggctgttscttcttttgtttctatgtcgatattgggaccataactttgctacactctgaatgaaatcaacttgtatacttctatcataaacttgagtattatccattttaaaatatgcttttgcgcaggactctttgtcctctaactcgatgaaagcataacagtgtctatccccagtttcgtgatctttgataattttagcaaatat

SEQ ID NO: 33 - DNA, 401 bp, Lycopersicon esculentum
  Polymorphism (201)..(201): A=susceptible allele ; G=resistant allele
tcgtaccattttttgtcttaaggcttgtaagatgatctctaaagagtgttcatatcatattgtcagagtcaaatatttagactctgaaactcctcccatggagttagtctccatagtgaggaaatttcctaaggtctttcctaatgatattcccataactcctctcgaatgagaaattgattttgtatcgcattgctaccrgacacaaatcccatttcaattcctccttgtcggatggatctggccgaattaaaagatttgaagcttaaactcaagaatttactaaagaaaggattcattaaaccttgcatttccccatggggtgctctgtttctaattgggaagaaaaaggatggatctcttaggatgtgtattgactaccagcaactcaataaagtgac

SEQ ID NO: 34 - DNA, 401 bp, Lycopersicon esculentum
  Polymorphism (201)..(201): A=susceptible allele ; G=resistant allele
aggaagtttcttccgtaaaagttcaatggaggaatcatttagttgagggtgctactcaggagccaagtccgatagtatgttgatatcctcatctatttccttctactcctactctagtttgaggtaatgatttcctcttggtttgttttatgtgttgtgagtcatgtacattttatgtgtttccatgatctcattttgttrtgcatgttcttgaaaaagcctatgttttgaaagaaatgacttatatgatttattttcctcatgatgtgtgcatttagtatgagttgcctatagacttcataatatgaatagatgaacatgtataagataaactttttgagaatgaattgtttaaaggctccatgagattgtgttgagcgtgttttagcttggagaagtta

SEQ ID NO: 35 - DNA, 401 bp, Lycopersicon esculentum
  Polymorphism (201)..(201): G=susceptible allele ; A=resistant allele
gaagtgagtcttacatctttttgttcaactctttttttctttcttttctctttctcttttttttccttgtaattgataaattttcgtgagtcacctctcttgattaatttttctattttttcctcattacttttttcatcccgactttttgggtcatcactcataatagccaccctccacttatgaatttgccatgagtcragctacatatttcccaaggttaggtcaaggccaagacaaggccagtttctgcattaacctctccacttatgattttgacctaagttgagttgtacattttcaagtagggaccacggccaatacattaaactcatggtgggtgacttggtaaaaaaagaaaggtctattataggttcaacaactttggatcaaaaaatgat

SEQ ID NO: 36 - DNA, 401 bp, Lycopersicon esculentum
  Polymorphism (201)..(201): G=susceptible allele ; A=resistant allele
ggatgtttggactagacccatgatatcctgaaaaacataaaagaagatggaatccttcggaagcaagaaccctcaccatagttaacttgaactccctattgtatcaacagagctcctattgatgatcatgaataattggttcttcattataaaatgatgtagataaattgtcttagtatgtgaaatatacgaatatttaargggaattcaaaagcgtaaacataagattgaactggtgcaatagaaacacacttaactcttctaaactaagtcgtctcaacgaaacatagttcaattctactcaattatatgatattcaaatcagtgaaagaagagtcaaatcaacaataagatattcaacgctaggtactcatcgatcactagtttttccacttaggata

SEQ ID NO: 37 - DNA, 401 bp, Lycopersicon esculentum
  Polymorphism (201)..(201): G=susceptible allele ; C=resistant allele
atgcacccatatatttcccactcactttggatgaaaatgacgttgaaagatatttttaagtcgcatgtattcaatggtaaaagatgtgcaccccgtctatttcatgttaaagtcaggagagtctgacatagaatgatatttttatgttacttttattcaaatgtggatgatgtgcaccttatagaatacgtattgaggtasgaggagcccgatgttgagggatattcatagtaaattataggaattagaagtctttacatatatctaatcgatgaactatgatatgtcgaattgatggacatgatttatttttcccaaagatactaccagttgctcatcttattctttctacagatgtacatacattcatatctgacgaatggattatcttcattcatcct

SEQ ID NO: 38 - DNA, 401 bp, Lycopersicon esculentum
  Polymorphism (201)..(201): A=susceptible allele ; G=resistant allele
caactcataccccaaacaacaaatttcatgaaagaagaaaaggaatgaaacaaattgctagaatgctttatagcttcctgaagattagatgtggactcaacacatcattctggatgattctattgcacattttctcttctactggagaccggtaacctgcgctctcatacaaatttttttacgacccaacccactggaccrtgcgggaacctactctaatatcttaaaaggagaaccctattttcctaattgttataatatacagaagaataagaaaattaagatatagaaataatttcatatagatttatataaataagaatttaaatttttcaaaattaaggcttgtaaagtaattaaaaccctactgatctagtctaaataggtatattatccagtaa

SEQ ID NO: 39 - DNA, 401 bp, Lycopersicon esculentum
  Polymorphism (201)..(201): G=susceptible allele ; A=resistant allele
ctaatttaagcaaatattaagacgttaatcacgagatgattagtcgcaacaaattgaatgaaaacagattatgacccaattaatacaactaaattaaacgcaaagattttcaagtatgacatagtgatgacaatctgggcaaaatttaatcaaccccaatatgcaaataatagagtaaaaagagcaaatattgtagggtcrtaaatgcttagaacaaggttcataactattattcaaagaaatcacagaaccatagatggaatttaggaaaattttgtgagaaaagtagatagtaatttaaacaaaagcaaacccgaacggatctgtcgagatccccttcagatgaaaactctagaacttcatgtgaagctgatgactgatgtggtggctcatcgctgttg

SEQ ID NO: 40 - DNA, 401 bp, Lycopersicon esculentum
  Polymorphism (201)..(201): A=susceptible allele ; G=resistant allele
ccacttcccgtgtagcctcttcttttttcttgttcctccaaagtattttgaccaaagctatgtctctactccttaacttcatgacttgtcggtccagtatagccactgggacttcctcaaatgataggtcttgtgtaggtttagtgtcttcggttttgatgacatgggaaggttgtactaaaaatgcataacatagatacrtggaagattgggtgcactgcagctaactcaggaggtaacactaactaatacgctacctggccagttaagcataaaattttataaggcccaatatatctaggactcaacatatctttccttcaaaatagcataacacctttcataggtgacaccgtaaaaaatacaaaatcatcaatctaaaaatctaaatccctacgccg

SEQ ID NO: 41 - DNA, 401 bp, Lycopersicon esculentum
  Polymorphism (201)..(201): A=susceptible allele ; G=resistant allele
ccatactacccaggcccttggagcacttagacttactccgatttggccgaaatgggcatgttgatgtcttttatccttaagaatgacgtcaattgctccataataggggaaaatatgacatttggcacaatacggggttactcagacttcaccctactagaccaaacaaatattcaacacaatacaataattttgactcartttgaacaataaaagaaagggaatcatgattaaagatcgataaataatgatttcacttaaagttaggcaaaggaatagtcacacgatgattacccagtgatattcaaaaacaaaatacagatttcattcgagttcaaacacatatagagtttgagtttggaaaccaagcttttttgttgaaatatatttgaaatgttagg

SEQ ID NO: 42 - DNA, 401 bp, Lycopersicon esculentum
  Polymorphism (201)..(201): G=susceptible allele ; A=resistant allele
ttttttgttgttggcgaaaagttgttgccaattctatattttgttgtaatggacatgcatgcatacacgatttacttaattttaggttactcatgactactttcatggatggtataacctctccatattttgtaatgatggctaagcctcctcactcttaagggtgtgattatgggattggtattgatttaacttagtggrtttattgaattgctctattttatggtcatttatcatgcatttgaaaattttgcaatgtgtttaattcataaattgaggtggccaactgatttttgtatcgatcttacattgatgcttgaaagtgaccaatatgagtgggtgattagttcattgtttgataactagtagtaaatccaaggtaatggattggatttaaaatt

SEQ ID NO: 43 - DNA, 401 bp, Lycopersicon esculentum
  Polymorphism (201)..(201): G=susceptible allele ; A=resistant allele
atgatgtaatgttgttgttattggtggtggtgttggaaggctctacttctacaaaggaatccattagtttttttatttttttttgagagttttttagggtttaataagagaaggacatggtgtggaggttgaggttaattgttgatttgaataatcaaaagatttctatatttgtaagatacaagatagagactagagaargattgaaaattggagaggcaaaaaaggaagagtttgatgaaaggattgatgggagagtgaatgaaaggattcctttttctctctctttgtttagctacgataaaaataacttttaacgataatatatatatatatataaataaaagtactaaatcttaacgatattagtcaatagtcaaaggattcaatgttaatataat

SEQ ID NO: 44 - DNA, 401 bp, Lycopersicon esculentum
  Polymorphism (201)..(201): A=susceptible allele ; C=resistant allele
tgtgcaatcaaaatacaaatccaatgtaccatgttttattgttattttttgattctcattctgcgtgcagagtccatattgaagctccgatttaatatgaatcaccactataggcaattcaggggtgacttattttctcacaatccaaaactcaaatttgaaatattaattaagaataaaacaatttcactaccgcaacgmtacctttatcaaatataacatatttatagagtctaacaactctaaacttagagtccaaaatccaaggccccacggctatgtaaaccgtacaaacaaacccattgtctcgtccacatcaaaactaggcaaaccacaaacgggtcgaattgctaacacactcagatccgtgggcctcattgctcctaccacttaagataata

SEQ ID NO: 45 - DNA, 401 bp, Lycopersicon esculentum
  Polymorphism (201)..(201): A=susceptible allele ; G=resistant allele
caaatccaatgtaccatgttttattgttattttttgattctcattctgcgtgcagagtccatattgaagctccgatttaatatgaatcaccactataggcaattcaggggtgacttattttctcacaatccaaaactcaaatttgaaatattaattaagaataaaacaatttcactaccgcaacgatacctttatcaaatrtaacatatttatagagtctaacaactctaaacttagagtccaaaatccaaggccccacggctatgtaaaccgtacaaacaaacccattgtctcgtccacatcaaaactaggcaaaccacaaacgggtcgaattgctaacacactcagatccgtgggcctcattgctcctaccacttaagataataagataataatagtat

SEQ ID NO: 46 - DNA, 401 bp, Lycopersicon esculentum
  Polymorphism (201)..(201): G=susceptible allele ; A=resistant allele
attatatcatattagtaatataattaactatattgtatcgtatatcgatagttaaatattaaaagttcaatcgattaactattttaccgttttttttataaattagaggtggcaaagctgaactcgagcgaatggtacttcttcagttttcgagatcgaaaatacgctactggttttcgtacaaatcgggccacaatttcrggttattggaaagcgacaggcaaagatcggacggttgtagatccgaaaactcgttcgatcgtagggatgagaaaaactctggttttctacatgaatagagctcctaatggaattaaaactggttggatcatgcatgaatttcgcctcgaaaatccacatataccccctaaggtaattcttccttcattttaatttattta

SEQ ID NO: 47 - DNA, 401 bp, Lycopersicon esculentum
  Polymorphism (201)..(201): A=susceptible allele ; G=resistant allele
tatttatggagttcagatatccctctcctaataagaagtctgaggtgtaacaattctcctctaaagagagctcaaagaacatagagcataaagctgagattgcaaaaggaagtattgatcttccttcgtatgtaggtccttataccatggttgggtaataaacatctaaatacacaaatctagatttttgcgattttaaaraggaaaagtatgagcactgcgataaggttgtgaaggtattgtctgccaatgataacctggatgccattcctagattctatgcaatcatcaagaagattttgtctcctgcatttaaattgcacataacaaggttagttctagagcctctaaataaaggttaaaccaaatgactatatgagggatttctagcttcttgtact

SEQ ID NO: 48 - DNA, 401 bp, Lycopersicon esculentum
  Polymorphism (201)..(201): G=susceptible allele ; A=resistant allele
aaacatcaaagatcgccaattgttcacatagtttagtaaatgcatgttttggttgaaatttgtggtatttttaggtcacatagtatctagcaaatgtatagaggttgatacaaagaaattggactccgtagagaaatggcatagaccactattgcagatcgatattaggagattttttggtttaataggacattattggargtttgttgaagtattctcatatgttgcttctcatttgatgaccttaaccaaaataaatataagtttgagtggctaaaagcttgttagaaagtcttccatgaattgaaggataggaagcttgtgagaaagacttccatgaatagaaggataagcttatctccaccccagtgtttactttaccggagggtactgatggcttt

SEQ ID NO: 49 - DNA, 401 bp, Lycopersicon esculentum
  Polymorphism (201)..(201): A=susceptible allele ; T=resistant allele
ttgttttgggttttgcattatgaaagttgaatatatgaaaatgtgcataccttctctttatgaaaatgatgtaaataaatttgatgatgctttcacttttaaaatatcataagtaacatgaataattaaatgaatatatgatcgtgaatgtatgtttatgactcaagaaagattatagatgagagtgtgtatatgttctawgaagctttgccacatgacatgaatcacacatctagacttgccttcaacgctttttctgctgttttcttaaattatgtcttaattaggatattattcgtatatgtcaccataattataagtcacgttttcaatattatttccactagtagtaaagcatctctaacatgaagtgaatcaatctgggccttaaaagctctttt

SEQ ID NO: 50 - DNA, 401 bp, Lycopersicon esculentum
  Polymorphism (201)..(201): C=susceptible allele ; A=resistant allele
gacaaccatcagtatgagaattttgtttatacaacgttatacaaatattgccagagtcgccctatacatttaaaaagtataaaactgaactgaattgatggatcgactaaaacactttcttgaggcaggtgaggagccgcccgacttgcggtttgaatctatctacgattgctgcatactttatgattatgtaaaatactmaatcgatttagataattactcaaacttgtgtttttacaaggtaactcaagtgtactcaatctctatgaaaaccctttacatttcgggaagtagtacactcttttcaaagtgaaaaaaatatctttttaagggtaaactgaacttctctttgtctagactgaaaagctttgaaaaattgtttattctcttctagaaaatag

SEQ ID NO: 51 - DNA, 401 bp, Lycopersicon esculentum
  Polymorphism (201)..(201): G=susceptible allele ; A=resistant allele
gacttttgtagagataccaattaaacgattaaaagtcctaaaactggagaaaagaaactagagtatatcaaaaggaaaaatacaagtatatatatatatagaactaaaataagtaaaatacatcatgtgctcacccccctgtccggatcaaaatgctccacactagatcaatgttggctactggtgctcagactttcatcrcaaaaatagatgcagagcgtagcatgaaaaccaaacaacaggtaaccagtaagaatactaaaatgactaaacttgaaattagaagtaaaacgaaaacaaggaatagcaagaccataggtcaagaacagaagtttaagtaacaatgaaatgcacacaagtgtagaaagaactacaaaagtaatgcataagctatctacaac

SEQ ID NO: 52 - DNA, 401 bp, Lycopersicon esculentum
  Polymorphism (201)..(201): G=susceptible allele ; A=resistant allele
tcaggcactttataatctctaggtacttgtcaataaccccatctatagacacacatttttcatcaagaatcatctttaaccactctgccttttatcttttcattgactttggcggaggaccatgtagtggatgtgccaacactataacctgcacaggtattggaggagtatcagtggtagggtaagtctttgtgatcctaratggatcatttagcttcttagtttattgttcattcctttgagttattgatagatcatccttatcagaacaaacaccatcacattctgatttgtgtgggtcatttttgtaatagccatcactgtcagtaatagaatctgataactcaaagatctggtcttagtctttgcctttctcatcagttttagaaagtttaatagcc

SEQ ID NO: 53 - DNA, 401 bp, Lycopersicon esculentum
  Polymorphism (201)..(201): G=susceptible allele ; A=resistant allele
ttgctacatatgactattctatggcaagatgtaattcatatgttgatacataaaattgttatgttattacaaataaattacatgtgacaacataagtaagtcaaatattgcaacatatgactattttgtgatgaaatggaagtcatgtgttgctacatataactgttcgattgtgacaaataaatcacatgtgtcaacatrtgtaggttatatggtgtcacatatggctttctgttgtgaaatacaaatatgttattacattaatatataaatcacatgttttgataaaatgtataagtcatatgttgcaacatatgtctattctgtcgcgagtaaaataaccactcagttgcaacacatataaccatctgttggaaacacgaatgtgaaatattgaaaaa

SEQ ID NO: 54 - DNA, 401 bp, Lycopersicon esculentum
  Polymorphism (201)..(201): G=susceptible allele ; A=resistant allele
agaatcaattggagcaacttacgtccctttcatcgaccaacagaacatcgattccaccatacctcaacacctagcaaaaccattggtggaagagtacttacggaagaaacaacgggccgtacatggattgaaggggcatcgatgcctccgtattcccaaacttagcctggtggtgaaagtccccaatattacaagtccctraccaagacgacggatgtgcagcacgaacgaggggcattccttagagtcaccaacggaccgtcgacggggtctcgttagtgaggtcagttaactttgagtcacattctatttgggtcatttaaattagttaggagtttagggggttaattaagggattaagagaggtctaattaagtttattagtcacccaataaagtcca

SEQ ID NO: 55 - DNA, 401 bp, Lycopersicon esculentum
  Polymorphism (201)..(201): G=susceptible allele ; A=resistant allele
gggtattgctcctagagtaaacattgttgagagagatattacatatttattgagatattttgtgagaatgaatcctcctattattcatggctctaaggtttgagaggatccctaagagtacctagatggtatgtagaaggtgttgagtgacatgggggtgacttctagggagaacgctgagttagatttgtacaaaatgarggaggttgctaaagtgtggttcactcaatggaaatataataggccgctagagttgatccctataaagtgggaagaacttaaagaagattttataggaaagtactttccacatgagaagagggaggtgaaggtcgaggggtttataaatctcaagataggaaatatgagtgttgactaatactttttaaagttctctatga

SEQ ID NO: 56 - DNA, 401 bp, Lycopersicon esculentum
  Polymorphism (201)..(201): C=susceptible allele ; A=resistant allele
tcttaggaaatgacctatgatgctgcccaatacaggtactcttgtttattttgtagaacttgtcatgttttattttggtatcatcttgggtatggattagctttatgagtgttttgcttccacaggttgtagaacaaggtcagtgtagttccaatttcctaatgaaactattctagagtggaaggcatgaaatttgatgcmaaggggtcaaatcatttcttctctgatgtctcgtatgatcatagccaaggttttttataccatgtggtgctggtaaggacctagaatgtcaaacttcttttattgagtcaggcctatgcttagagaatacctagaagtttttcctattgaccttccaagagttcctccaaaataagaaattctcttcggttggcatttat

SEQ ID NO: 57 - DNA, 401 bp, Lycopersicon esculentum
  Polymorphism (201)..(201): A=susceptible allele ; T=resistant allele
ttttagttaaattaattaactattcttcccttaatacctaaataatcacagaaccacttaatttattatatgatttattataaaaacatgcaggaaatttaaccctcacaggcgaaacccagttgacaatccatttgtgagtcaacggttggtgcttgcctccgtccacaacaatcgttgtcttggtcagagactggtgtwggcaagggcttcctccatttttctaagtgtggttctacggtttgcaattacaccccatcgatccatacacaaatcgttgtctgtatctcgtcgatgcatatttcctaggatgtgatgacatcaactttctacgatcctcctcgagggccattgattggtcattgtggagtcatatccggacgttttgaccatgaatcacc

SEQ ID NO: 58 - DNA, 401 bp, Lycopersicon esculentum
  Polymorphism (201)..(201): G=susceptible allele ; A=resistant allele
atgacttaaaattctttgattgaagatttaatttacgattttgtaagaggtagttgaattggctaaggttgcattgtgtagtgtcggttgggctgcatataagtaaaagaaatatgattggagaagaggtgactgatagtcaaccaggtcatcaggacaatatagatgataacaatagtgcttataagtcatatcatgctrgcaaaacgggtgctattttcttacaaaaagatcatggcaatgtaacatttcaagtcactagcatgagtctttatctccttaaaatgaaggaacattatcgagggctagaccatgaatacccgcatcagcatgtaagaaattttatagagatatgcagcccgttttaattcaaggagtcaacccggctttggttatttcaa

SEQ ID NO: 59 - DNA, 401 bp, Lycopersicon esculentum
  Polymorphism (201)..(201): A=susceptible allele ; G=resistant allele
aaatttctatttgttggagaagagaaccatcataggcaaatgattagttccattatacagccactactaaaaggtattgtgatactcttaccactatcataggcaaacatatgcgactcgcgtagcagtacctacaatcaaccccacaacaagccaaaaggtttttccctgaacaacttcaacctttccttcaatattgcracttctaggtccatcaacattccaagttcaaagcagtaaaacaacgggagaaaaattacacaactacaatccaaaatcactagagtcctccttcaacacaataaaacactaatacaacaaaaacaatgtatagacttaactttcaaagattcaagataatataataggttcagaatttttacctagaacaccaactcaag

SEQ ID NO: 60 - DNA, 401 bp, Lycopersicon esculentum
  Polymorphism (201)..(201): G=susceptible allele ; A=resistant allele
atgcttgtgatattccttgtatatctaacatatctgtattgctatgctcatcatgggaggcaacaatatctaataactcaaaatttgattttgtatcagtgttggggttaagtaaggcttcttcgagctttctttttaatcttttttagtcataatccttttgagatgatgaacttgtacttaacttatcatcgtagacarctagagcttatcctttgatcttaatgattttgcttaaatatttgatcctctctaattcatgaatattgaaggaccaactaataatataagagatctttttggtgataaagaattgcataatttaatatattctgataaggtaaatatatttctctctacttgaaacttctcaaactgtatttgatgttgagttgacaata

SEQ ID NO: 61 - DNA, 401 bp, Lycopersicon esculentum
  Polymorphism (201)..(201): G=susceptible allele ; A=resistant allele
aaacactataggaaaaaatccttgtacttgatctgccagcaataacagcttttatgtcctgattttgaaactgaaatttctcattcttcaatcttctcggggatagaccttgtttttgagtcacacattagatttcctcttccatgtcctgattattttaaggagtaaatcagtagtctaatcatcatcatcaagtttctrtcaatattgattattcatgtatacttcatattcttaattcataacttcataatcatcaatcaaagggacaatatttacttggcaattgccatgtactttatcaggaggtcaactaggaggaggttgttaaaatattggtctgctcagaatgttacgcgtacggtgttgattatcctaatcatcaagttcatgtcttttat

SEQ ID NO: 62 - DNA, 401 bp, Lycopersicon esculentum
  Polymorphism (201)..(201): G=susceptible allele ; C=resistant allele
ggagggtgacgattctaacaaagaaactaaaacagatcaataacctcaaagaggaccttggctaaatagtaacaatcatgagtaagtgtaggaagatactagaaagcatggattttctcataggggtagaaaagaatctacgaaaaataaaactgatagttttaacagacaacatagtagggctaggagtagaggtgtttsatcattaaagccatattttttaatgctaggagtataaatacagttggtgcatttgaaagactataaaaccttaaaaaacttcataatttgactatgattgatgtgttggaaccttttgcaaatcattggtatattaaatcaattatgtgtcatttacaaatggatcatgcttattggaatgtcaataataaaatttggtc

SEQ ID NO: 63 - DNA, 401 bp, Lycopersicon esculentum
  Polymorphism (201)..(201): A=susceptible allele ; G=resistant allele
caactagactagcttccggaggtctttgttgttcttggacgtttcactttcaaaatcctctaagtgacttgtaaagctaatattcaacatttttagaccttacaacctcatgaaacatatattaggctctatttatacctaaatcattttgggttcccttcagttatactttatggacccaaattttgaaaaacttaagcrcatctcctaccctaaacaagtatttcaacattcaatcatgagaaattgtcatactgaggcttccaagtacttaattttaaatttgaaccttaagtcaaaacttaaatacagatcatgaagttcttttttacagtatgaacagactatctaacttggtgttgctaattaaacaatcgcatatagttatggacaatacaaaa

SEQ ID NO: 64 - DNA, 401 bp, Lycopersicon esculentum
  Polymorphism (201)..(201): A=susceptible allele ; G=resistant allele
aatttgaaccttaagtcaaaacttaaatacagatcatgaagttcttttttacagtatgaacagactatctaacttggtgttgctaattaaacaatcgcatatagttatggacaatacaaaaccccatggtcaagacccactcctgaattccaaaaaatttaactatgaacaaatattcatgttttctaaaggaaattgttrccttagacgagacaaggagtggtgtgaatgtgaagcgacactaagtttccaattaagaacgcacaacttcactgaacccggacaccgaagtaatatgggaatggaggttaagaagaatttgtgtgacttggatgatatagggggttttggggatgatatgagttggagtagggattgattggtgggaaaaaggagtgaat

SEQ ID NO: 65 - DNA, 401 bp, Lycopersicon esculentum
  Polymorphism (201)..(201): G=susceptible allele ; A=resistant allele
ttcctgatcactgtcattgtcatgttgcttacattgtctccttttagccgcatcctttttttgttttctaggcttgtttttactcttgggagtcctttccagagtactagtatcaatctacttccctttatctttatttttcactttttcaagttgtttttttttaaaaaaagaattatgtttcttgttatccctgttctrcacctcttgttgactctgttgttaatctatttgttgattcctatttgtagaattatcaatggatctaaggtttgaattatggtcagtatcgaacctctcatacaaaaaaagaggcaaactccctctttggtatccccttcctgcatgttagtggttttctcctatcaccccccatcaatacctccaaccacttcatcagt

SEQ ID NO: 66 - DNA, 401 bp, Lycopersicon esculentum
  Polymorphism (201)..(201): G=susceptible allele ; A=resistant allele
ctctacagcctgatcattcgaaggacttaaggctttaattagagactcaatagtatcatcatcctcatacgtatcatctagagaatggttgtccctatccaattcatcttcagaaatattcacagcatactcatcaatcaaaggtattgtattaacttgacatatctgtttcatcttatctggtggttctattgacggtgragtcatatatctagtctgcaaaggtataatatctaaacctttgttgtcattggccatcacaaatttttttcatggttcatgatcctttcctaagcttgttactattcactgttctgctgtatactttctctcctattagcatcatcccttttttgtttgctttggcttattttttctcttgggagtgctttcaggagtac

SEQ ID NO: 67 - DNA, 401 bp, Lycopersicon esculentum
  Polymorphism (201)..(201): C=susceptible allele ; A=resistant allele
tgttataaaaaaaatgagatcatctcagataatattttcagtatataaccagtaatatcatatccagtaatagaattgtcccacatgcaagttaaaaaggttcagtaacacatttactttatataccatgtgaaaatcatccaaattataaaatatcaattgtggtaagattattactcacagtacttgtgttgatgcacmaaacttgtcagagaaatcaataaggtaccaaactcgcagagaaatctataaggcttccttcgataaatcacaatcacatgacatggatctcgagataattatttgctaagactaattcaaaactaagataaatattattttaaactcttttggactccattttccatcaaatctctaaaataaaattattcaactcataa

SEQ ID NO: 68 - DNA, 401 bp, Lycopersicon esculentum
  Polymorphism (201)..(201): G=susceptible allele ; A=resistant allele
aaaaattttaggcatgggtagctacctgcattgcggttcttgttatcacctagatatactcttccagacaccttttcaatatttacctaagccccgaagaaatgacctttagcaggaaaaatagagaaataagttattatacaggaaacaagattgcaagacaatattcaattagaagttttaccttctcaaatctttctrcgtacactgtgtgaaatcaaagagaacacacaaataagacatcaaaacataaaatgaccatactcacaaaaaagcagagaaaataaaccctcaaaaacggtacacaggaaaccaacatgtgctcctcttctgtccaaggagtccctcttcgccatttaatatatgctttcatattttggttgccattgttttgcaagttt

SEQ ID NO: 69 - DNA, 401 bp, Lycopersicon esculentum
  Polymorphism (201)..(201): G=susceptible allele ; A=resistant allele
catcatatgcttgagagtcttacctttatcattgacaagtgatgttgcagtgtgattttctaagcttgcatataactcaattcatacattggaacaattgcatacggtgttcatgtaaatgtacttcccgttgtctaagaagttgaaacacaaagacaaacttaacaacttcatggaactacctggagaatctgtgagtarttctggtacaagttcattgcattcataagaagtgttttgaatcaccgcattgattatgagtcactcaaagagtattttggttaaggacaggatgacaatcgtaagaaaatgcttgatactatcatggaggatcatatggtgaattcacttttgaggagatcggtaaaaaattagagaatatttcccaaaataataaagta

SEQ ID NO: 70 - DNA, 401 bp, Lycopersicon esculentum
  Polymorphism (201)..(201): A=susceptible allele ; C=resistant allele
tgtggtacatcaaaaccctcaactaagaattgttggtgtcatcaagcaacatattgagaatctagacaacacttgtatagaagcaacaaatttaaaaccaagcaatcatatgtctctccatattgaatcttattttaagtaaagttttgttctcttagacgcaaacagtctaatttttttaacatccaaacaagacataamaaaccaataaataacaccaaatataatgtttctctatccctagagtaccaattttcagacaacgtcaccattgcccttatttcaacagaattcccctttttcgctcaatgtatatgaagatttagtatagggattccttcaactctcatgctcagaagtaattatcaaggacatgtaattctcaataccataggattgac

SEQ ID NO: 71 - DNA, 401 bp, Lycopersicon esculentum
  Polymorphism (201)..(201): G=susceptible allele ; C=resistant allele
caaagtcatatagtaacacaccattccacaaaagactcattgaaactataacaactcctaacatggtttaaccataacatgacatacatttcccaacataaaataccacttcacatatatctcatatatacccacaggggaactatataaaaataagccaaagttaacttttacacatactcatcaaaccataataccaasataagcataaaaacatacattttctccctcaggcataaagataccatctcatccaagcaaacacatctacaagaccacaggacaagaaaatataagagagagatcatatagatttaacttctttgtcacgacatgagaatgatgaaaaagggaaaatctatgttctatatcatcacgctcatggattatcagtcaccacc

SEQ ID NO: 72 - DNA, 401 bp, Lycopersicon esculentum
  Polymorphism (201)..(201): G=susceptible allele ; A=resistant allele
ttctctttgagcaagcgatgttggcaaatgaaaggcagtgtaaccctagtcttgcacgtataacatatgccatgtgcgcattggcagggcaacattagatcgaccaagggcaggcaacatcatccaagtctattgtgcttcaggaaagagacaacggtcaatgtcaagtagtatcgtcaaggtgaccttagttatggaatrtcacattagttgttgatattgcacgtcgctcaatcaagggtaggcatggatcgctccatcatctgtggtctgtgcacactactcagcagttgttaggcttggtctttctgcatcgcccttggcctgcatacaatcactagacaacatcagatgtgggaggcactcattttttcatggcctgcaaattgctcagccgacta

SEQ ID NO: 73 - DNA, 401 bp, Lycopersicon esculentum; identical to SEQ ID NO: 72
  Polymorphism (201)..(201): G=susceptible allele ; A=resistant allele

SEQ ID NO: 74 - DNA, 401 bp, Lycopersicon esculentum
  Polymorphism (201)..(201): C=susceptible allele ; A=resistant allele
cacctagaccatcaatatgaataataggcgcgggataacagatacacttcttaagaatggataaacggaaaacatgatgaatcgaatccaataaactaggtaacctcaactcataaggaaccttacgaatatttttcaaaatttcatagggacacacataactagtactcaacttacctttcatgccaaatctcaccaccmctttcatagaaatttttttttgatacacgttatcattttcttcaaactctatatctcctctccaattatcatcataagaattttttaaactatagtctgtttgcaaccggttcctgatgatatgaactttctctaaggtcttttagataaaattgggaccaagaagcgaagactccccaaccctaaaccacacaacctga

SEQ ID NO: 75 - DNA, 401 bp, Lycopersicon esculentum
  Polymorphism (201)..(201): A=susceptible allele ; C=resistant allele
ttataacttagatgacagaaaagatgttccgtacctctatgatctcttgtttgggatttttttattgtttggtttgtgaattggtggaggttgtggattttacataacaaaagtttaatagaggagttgacaaagcgatacatttgtatgtgtgcaaccataagattttaaaggctgaaatggtgtgtgtctgagtgaagmaaaatcaagattcttaagtaaaaaaggagggcgggtaaaagaaacaaaaaaatgatgagaagtactccctccatcactttcacatatattctttggaattcacacacctcttaaaaaaatgatttaaataagttcttactattatacccaaaatgttccattaacctcgttaatggggttttcttatcttcactggatta

SEQ ID NO: 76 - DNA, 401 bp, Lycopersicon esculentum
  Polymorphism (201)..(201): A=susceptible allele ; C=resistant allele
acaaatgctttgtcttaacacatactcataggtcatcaagaccttcacattatgcacatgcttaagttataatacgataataccctaattaataaagtaatgttcgatgagtccagatagtttcaaaatatataaagtaacaccgacgatgccacattgtttcacaagcatagcacataaacctataatatatattatagmgaaagataggtcataataccaccaataaaatctcacaactttactccacaataaatcaataaacttaatatgccaaggctcttacccatagccatgattactaattcaatcaaataattcaaatctacaattaattgaagatatgaatttatcattttaggaaaccttacttacaattgtatcataatttttcataattc

SEQ ID NO: 77 - DNA, 401 bp, Lycopersicon esculentum
  Polymorphism (201)..(201): C=susceptible allele ; A=resistant allele
attttttaataaggttcacacacaatataatatatccttaatcttatattgaatccttggataaatagtcctaatctcgtggtccctattcttcttcttctcatatggtgttcgtgagagttccaatgagagagacatgggttttgatttgaagtctagattctaaggggttcttttattttttaaatagtcataaaacamctaaaaataccttaagtaaccacatggtttgacttttccaattcactcttttcacttaaaagtgaattgtgagaaatgcaggctccatagactatttgacattgatcgaccactggtttgaaaattgtccatcctgtatatacgtagtcttggtaagagacttctacaaggttcccttttctcaggagagagtctcaatt

SEQ ID NO: 78 - DNA, 401 bp, Lycopersicon esculentum
  Polymorphism (201)..(201): A=susceptible allele ; T=resistant allele
gcatatatgtttgtatttaataataatatgaatcgtaccaaatgaaatgaatgaatatgaataattgtatcccatgaatcaacatttgattgtttaattatgagaattaacaatgtgattgtgattgttacatgtagaatacattaggtatttcgttccaacacaatagctgggagcggatatcaggtatcgacatatatwttatactgggcgtcaaaatccatcatactaatagtttgggtgtaggtttaataagagtgacattaacttgtcatataagcatattgtatttaatgtgtaaatggacatcttttcataaagataattggattttatattatcttcttatatgtgacttcatagtgttgtaagctaattgcttatatctcataatgaatgac

SEQ ID NO: 79 - DNA, 401 bp, Lycopersicon esculentum
  Polymorphism (201)..(201): T=susceptible allele ; A=resistant allele
gacttacactattttttttggagaggctacatgtgtgtcaatattgtagcttgaataaaaagaaggtaatatgtagtttttaggtgtgtaataccctatataaaaatatataataagggcatatatgtcatttgatactattttaaaaagaactaagttgaattgtttttatattgacctaatggagttgtttaaaaaaawtgggctataaccctataggaaaagttgtacctggcaacaaataaaaaaagataatggggatcaagaggggttcgctgcgtacatcaccaagaagaaaaagaaaattttgaataagaaagagaggaagcgtgaaaaggagaggggacatatgtagaaagaaaagcaaagaattcaactgaagaacttagaggggagacttt

SEQ ID NO: 80 - DNA, 401 bp, Lycopersicon esculentum; identical to SEQ ID NO: 79
  Polymorphism (201)..(201): T=susceptible allele ; A=resistant allele

SEQ ID NO: 81 - DNA, 401 bp, Lycopersicon esculentum
  Polymorphism (201)..(201): C=susceptible allele ; A=resistant allele
atgaacccccaactctgtaacagagccttatggtataaatgtgtacttttttaaaaaatgtttgggatattataaatgaagacttgatgggagtggaattagattttttcagtgacaaggagatccccatgtattttactcattcttttatagtgcttctactcaaagtgaatgatccttaaaatttgaaggagttaaggmctattagtctgagcaacttcattagtaaaatcatttccaaactattatgcacaagattgagccctattctgcctaaattaatctatctcaacaagtatggttttgtaaagggaagaagtgtaactgaaaatatcatgtcggcacaggaaatcacccatcaaattaaaaatactatcataggtagcaatgtcattatcaaa

SEQ ID NO: 82 - DNA, 401 bp, Lycopersicon esculentum
  Polymorphism (201)..(201): C=susceptible allele ; G=resistant allele
agcaaaatgactgacttatttctcatcctcctatgaatcaattacaagaagtggtcttctctatgaacccccaactctgtaacagagccttatggtataaatgtgtacttttttaaaaaatgtttgggatattataaatgaagacttgatgggagtggaattagattttttcagtgacaaggagatccccatgtattttastcattcttttatagtgcttctactcaaagtgaatgatccttaaaatttgaaggagttaaggcctattagtctgagcaacttcattagtaaaatcatttccaaactattatgcacaagattgagccctattctgcctaaattaatctatctcaacaagtatggttttgtaaagggaagaagtgtaactgaaaatatcatgt

SEQ ID NO: 83 - DNA, 401 bp, Lycopersicon esculentum
  Polymorphism (201)..(201): C=susceptible allele ; A=resistant allele
ctggcagctgttgatgaggagaaatctatattgctagaatgtttgaaagttggctagtaggtgcctgccctattagcttgcttcaactccatttcccttcctcctgaaattcaaccaattttgagttgttaaatctgttactatttgtggtgaattgttctaatggtccaattccaagccaattatctcaccaaaaggaamaattccaagcttgaagtttccattggatgtattgctctacttgttgtaggttcatcagcatgtatttccaagttaaagaatctccagtatcccaatttttgcttacagggttggacctttgttggtattttactctaaaaaattctccccataaggtttgtcttgttcaaaaaatccaccatcgtttgaattggaaggcc

SEQ ID NO: 84 - DNA, 401 bp, Lycopersicon esculentum
  Polymorphism (201)..(201): A=susceptible allele ; C=resistant allele
agtttatgtgatcaatataggctatattgatgccttatatgtctatacacaatgttgaattttgaatcttcctaattgactatatgaacactatgatgagttattgtgtttattttgtagaagtgtgtagggggacttggagtattctaaagtttaacctacgtgatacttgaataatattgtacatgtgataatatgatmactaagtgtgtcttgtttaggtaaaccgatatcccttacttgatacatgaaagaatgtgaataagaatatgcatccttgacttaataagaaaagcaccgttttgattaatgtatatgtatgaataaatatgatctttaatatagtaaaggtcatttatgtggaatcatttagaaagaagtttacgatatctttgatattg

SEQ ID NO: 85 - DNA, 401 bp, Lycopersicon esculentum
  Polymorphism (201)..(201): G=susceptible allele ; A=resistant allele
atttttagttcctttagttactccttttgacattatcaatttatagatttgcacttttaacataagacaagttcgtcatttctagggttccaattatattctattattaacctacttaagtcacaacaaggagctctaagttggaatatttagacagacagagacacggactttttagatagacttaatagactacacaarcgcattctatcattccaagggacatcactacactagttttagcatgcaatttcaaataatttagtccaaaggacaagaccaccctctgtatttctgtaacaaactatgaactagaattcaatgttaagggaaaacaaaataccatcaatgcagagtggtgttgttgtgagttgaactattaaggagtatcctacgcacac

SEQ ID NO: 86 - DNA, 401 bp, Lycopersicon esculentum
  Polymorphism (201)..(201): A=susceptible allele ; T=resistant allele
atcatcactacttaatgatacaaggaaatgttttgccaaattgttcaaaactcaaatcactaagtatagagcggaataatatatggataaataagtaacattgggcatgacacataggcaaactagaccaaaagggagcatgcaagttatacaagcattgcgcaatagaattacacggtgtactagtgcatatgacctatwgggtagagtgtgagcgaaagaactaaatcaaatgtaggaatggagtagatgatcacatgaatacataatttaagacatgtagactatatgacatgcagacatataaaaatgaactactgaccagaaatcacattgagtaagtgtattatatcgcataaataagtggaataacatggcaatgagtacacatatgtaatatg

SEQ ID NO: 87 - DNA, 401 bp, Lycopersicon esculentum
  Polymorphism (201)..(201): A=susceptible allele ; G=resistant allele
tcaaggacccttcgggcggtccttagggattcgtgactagtcgtttggaccctttatactatatttgacctatttaacttgtttttacaagttttagacttcatatgaaactcccaaacctccaccacaattagggagtctactacttcgatttagctattttctagacgtcaatgtttttagttttcattttggatctarccttttcagtaacatactttattacatcaaaataggcttggaaacatcattttaaccattatgtaaaagtcgacgcgcaagtctaagtcatggaacttcaagagtgttacacttaagcccccacacaacagtctatgacaataacaatggaccagcaggatggtccattggtaagttgatcttccttcgactgtatcgtc

SEQ ID NO: 88 - DNA, 401 bp, Lycopersicon esculentum
  Polymorphism (201)..(201): A=susceptible allele ; C=resistant allele
cttggggttctcattcactttctacacaaagaagataggagggttcatcctagtgaattccttcaagcgggaagccgtggtactagcattttgttaaacacggagtccaacatccctatgtacttgaacctccatggctgcgacttgtgatttggcagtcatagcttgatccaaagtaaaaaatgtggaccatatatcttmaatcatgaaaggtggaggaatgaccgaaggttgttcttgaggaggcacttagttatattgtggagggacttggttttcttggggaggaaatccctccttagacaagtcttccccaacttttctttcagctgcacgtgtagtagtcatatcctatatccacaaaaaaaatgattagaaggaaaaagacatgagaggatttt

SEQ ID NO: 89 - DNA, 401 bp, Lycopersicon esculentum
  Polymorphism (201)..(201): A=susceptible allele ; G=resistant allele
actttgaatgccttgtacggaccataggaccttgtatagtctgtacttgcccttgtttaaacttgtatgagcacactcttttgcacttttattttcaactgaacttttaatttcggaggtgctacaaaaggctcattcatataattatttattcactcaaatgaaatgaatgaacaatttttttaatcaataccattagcrctatatatcagtcggttcagctaagttttgaagtttatcgatttgacttatcgataatcagtttgtagatattctaaaccattgagatattttcattagtgcggttatcaatttatcaattaagtgtgacataaacaacaatctttgagaaattgcatatgagttgagaaattgcacttgctaagaccatctccaacctc

SEQ ID NO: 90 - DNA, 401 bp, Lycopersicon esculentum
  Polymorphism (201)..(201): G=susceptible allele ; A=resistant allele
ctagttatcagtgtgaagcatgccatatctataatttaggagactacaacactgcacaaaatttctagcttctttcacactcatttcgtgcattagagtttgacctccgaaaagtttcttcctaatacgtgcattcgtgtgtttttaagttaatcatgcctccacaaagagcacttaaaggttgtcctcctaggataaatrttgaggattaaggggtaaccaatgcacctgaagtgcaaccccttgtaaaagttaacaatgttgagttctctaagtctatctcgatgtatagtaaaggtgttactaaccatgtttggaaaaaatgaggggctagaaaggatgtagcgcaacttcggggatacgagagttctagaggataaatccttcaagtttcactctaa

SEQ ID NO: 91 - DNA, 401 bp, Lycopersicon esculentum
  Polymorphism (201)..(201): A=susceptible allele ; T=resistant allele
aaagtttctctaaatttgtaactacaaagaaaggaaactttgggtaaggaaactgacatcttttacatctctgcttattagttaaagagttacaactatagattcacacagacatttaatttgaagtgttccactttgtttaagaacataagtcatcaataatcagtagtggatatatctacagaatgcaataatttgttwactttgtgacaggtctatttgaaactccaagcacatgctgcagcaagaatgggaaataatgtcaagaactcatcagctttcctaaagtctccaacttctacattacttcatacaattagtgaatctgagaaggcatcatatgttgcccatattaacaactatctcgtgggagatgaatttttgaaaaaattccttcctat

SEQ ID NO: 92 - DNA, 401 bp, Lycopersicon esculentum
  Polymorphism (201)..(201): G=susceptible allele ; A=resistant allele
atttggttatcttatcttaatgtatccttgctaatgccatacattgacagaagaggtgctcaaacatgattttcagcaattattttgctaatcaagaacttggtttttgctcaatatgcaaagagtcctttacactttgtcctcacgtctttgttacgatgtattaataccaatccattcatgtgaacgatttctcaaatrtgtccaccatatctctctctcaatgaatgacaacgaatatgtattgaacatagtaggacttagaagttttaaagggaattagtttggcaacctctcgcgaactacaattatcagggaactacatgcaccaaggcaaaacagatccaaagaatgactgaatgttttttcgctagtgagaccttagggttatcaacaatttc

SEQ ID NO: 93 - DNA, 401 bp, Lycopersicon esculentum
  Polymorphism (201)..(201): G=susceptible allele ; A=resistant allele
aagatagcctagtagtggtaagttcctttgaaactatgtgtgacttccatggtttaaatccccctggctagaattttactttttttagtttttttttttttaagttccaattccgtttttacagtttttcttttaaaaattaaaacatgctaaaagtgtggttttaaatccaaaagaaatcaagggtcacatttcttgggraaaaaattgttggcttatttacacgtttatatttttattttttgaatccctgaatgaaaatcctgggtctgccactgctaggtgtgtgtttatcgacgcaagtcctaaaaaaattactactgctttcaaaactaataagccagaaaaaactgaaccaaatggacggtaattctttagtttggtttgatttggttttagaa

SEQ ID NO: 94 - DNA, 401 bp, Lycopersicon esculentum
  Polymorphism (201)..(201): G=susceptible allele ; A=resistant allele
gttttttggttcatctattggtgtatttatagtgtatatactggtagaattgttaaatttaggtcatatataaacaccaactagcatgatagttttttggtttatctatttgtgtatttaatgtataatctggtgtagttgatatatttaaggtcatacatagtcaccaactaacatggtagtttttatgttcatcgattrgtgtatttgaggcatctacattgattcagagaaaaagggcttgagttgcagcaacttctattcttaatggacaacaggctatcacagatgatagcctttttactaggaattcagatagtgtattttttggtgtagttgatatatttaggtcgtatataaacatcaattttcatggtatatttttggtccatccattggtg

SEQ ID NO: 95 - DNA, 401 bp, Lycopersicon esculentum
  Polymorphism (201)..(201): G=susceptible allele ; A=resistant allele
ttcttgtcatatctatagtctatagttatatgaacaaaagagttacagaactccacttctctacaatataggatttacaagatccctacttaatgtttttgcttaaggtcttttcttggtcttcgacttgctctgtgcgtcatctgtgagattgatcttgactctctattggcggcttgtggatcattctcaggtaacttragttggcggttcaaactctaaaataaacatcacaaaatgacgaatctgaatgggatgtgttttttttaggaaaagaaagatacgagaatccggaactaagacgtataatcctagattgaaaaggagaaataaggtgtattgccttgagagtttcaaatgagacgtattgtcattgtgggttgacctacgattatgttatc

SEQ ID NO: 96 - DNA, 401 bp, Lycopersicon esculentum
  Polymorphism (201)..(201): G=susceptible allele ; C=resistant allele
agcaaccacaaccgatgagcttagacagcaaagcatagttgtcttaagcagacaagtcttactttcatgacgccttgttagttgtttaggagtgttagtctaagctattagaacaacttgatctgtgtcagtcatagccatcaaaaaacgcttgatctcttttgttgaatcctatttgcatgaggttaactttgacacctscagctgaattattgaagctcttctacatgtttaggcttaatgttgttgatatcttaatggaccttatgaaactgctttgtgctaggctgtggatgaagcggaagaaaatgtttttctgttttctcgtggtatcaaaaatttgtgggtccaaggaaaaaaataatgtgatgttctgcgttcgattctctctattttgagca

SEQ ID NO: 97 - DNA, 401 bp, Lycopersicon esculentum
  Polymorphism (201)..(201): A=susceptible allele ; G=resistant allele
ataccaagttttacacgccctcaacattcactttggatgcgactgataatcaagaactattgttgaccacaagcgagcccttggcgtggcttaattcttagaaggagacttactcatgataagattattttaaaagtaacaataactcaaatgataacttagaatttttagaaataaaaattcaactacccaaagtggtarctcaagtaaacataaaataaatgaaaaggagaagactttagaactagtatctatatatctatctttgaaatctctaaatactatgagagatatagggacaagactaccaaccaccctaacaaaactgaataacaaaacataataaatggatcttccgaaaatcaaggaggctcatcaaatgactctgaactcgactggat

SEQ ID NO: 98 - DNA, 401 bp, Lycopersicon esculentum
  Polymorphism (201)..(201): C=susceptible allele ; A=resistant allele
actagaggctttatgaggagtcacccgaatcaatggcacgatcctatcctacggtggttgtgtagttatggggtataatttatctaaacactttcctaactccgtattcaatacaaaaactctcaaatcaatgcaatgattctcaataactgtataaggttccatgttagaaaaaaatgtcccaaggaaaaattaagaatmtctatcttgaattacaactcaatttaggttcgactctctgagtgttgggaagtgatcttttgggctattgtagtaatcggggaacttccacggatttatggattatgtaggtaaatggggccaaagactacatttgagttcctcaggatagtttgagccaatgtggtgtaataattttcctcgattctaatgttgttagt

SEQ ID NO: 99 - DNA, 401 bp, Lycopersicon esculentum
  Polymorphism (201)..(201): A=susceptible allele ; T=resistant allele
attatcataccagtgttgttgaaactgatttttgaaatgataatttcaaataggacaaatattttttaatgacgatttagacttgtcttgtaaaaatataatgttgatttttgatgcattattttggaaaaggggcattgataatatatgagaagtaccataattttgatactaaattagtggattttttgtggcactaawggggtacaatgatgtaaaatgaatttcaaaagtcattgatggatatgtgtctatccttaacagtgtcataagatgaaaatagctaagtttttgtttaggagtagactgagaacatttggaaataattgtgcatctagccacatatataacattatcacaccaaattaagagtgtccacgatttgtttcacaaacagatcc

SEQ ID NO: 100 - DNA, 401 bp, Lycopersicon esculentum
  Polymorphism (201)..(201): A=susceptible allele ; G=resistant allele
actcattcaacaccatcgtccttcatcagcttctacttttgagcgtactcatacttttgcgacccttcactctaaatttccttataatttcttttgcatacttcttctgtcaaatgaatatttcatcttggatttgtttaacctccattccaagaaagaaagtcatctctccgagatccgtcatttcaaagactttcatcrtctcctttttcaactcttctatgagcacaacattactcctcttactaaaagatcatcaacatacaaagaaattataatgatgtcagtgttaaaaatgctttatgtaaagagttgaagcacttaggcttttttcaaaacctaagtaagaaagatgatcatcgattctactataccaaacccttggaacttgctttaatttg

SEQ ID NO: 101 - DNA, 401 bp, Lycopersicon esculentum
  Polymorphism (201)..(201): G=susceptible allele ; A=resistant allele
ctaaggattccgtacgactttacagaattgaattcgggtgggtataactttcgaaattgatcttagagtgttttctgagtaacgttggttagtggtgcattgtgaaatatggaattgattaaatatatcactttttcgtgtaagcctccctagcactggattttatcaatctggcgggaccattgtggtggatcagatgcractttaagttgtaaatgaaccccaaatgcctttattctgtacttttcgactttggaatctaaggaatggccctgcaactttttgatagtttcccaccattcttaagtctaaaggtaagtgttcacctcccaaatccatttttctatttgtaaaacctattctcttgggttattatcaatgggggaggattttttaattgg

SEQ ID NO: 102 - DNA, 401 bp, Lycopersicon esculentum
  Polymorphism (201)..(201): A=susceptible allele ; G=resistant allele
gttactttaatatttaattgtattataataaattctatatactaagggatgcagttattttataattttgagaaaatacatggcatattcgttcatcttacttgtgaccttttttttaatagattatagtacattccttattaaatgggttaaagctatgatctggaaatctccatgcttaacgtctgtatgttttcaatrctatggtcaaacaatttgctcatataatttgtgtacgtgttatgaatttgggatttttcatatgagtaaaatagtttttaaaaaaaattctgttatgtccttgtgggcataggttcaaccttgaagtaaatttagggatcataataatgggtgttgttaattttgaaatcttattatggtcatttgtttgagtagattgc

SEQ ID NO: 103 - DNA, 401 bp, Lycopersicon esculentum
  Polymorphism (201)..(201): T=susceptible allele ; A=resistant allele
tgcaacttctattcttccttccgcaggtgggagctgtgtattttatttatcttgtctagttgtactcttattagcttttgtacttgtttaactagatccatgagagttgataaattactttacaagttttaagtaaatgtgtgtctaaagatattatttataaaatcatattattgcaattacttctgtttccttaaaaawtttcacatgtttaattattgggatacgagagttctcttacttaggtgttagagtggtgcctggacggctcggtgggttggatcgtgacataatatatgaatttgtcaaaaaaatttacattatttttgtgatcgctatttcaatataatatacagtaagcatgcatatatgtacaatggatatatttttgtcatgctcta

SEQ ID NO: 104 - DNA, 401 bp, Lycopersicon esculentum
  Polymorphism (201)..(201): C=susceptible allele ; A=resistant allele
tgcgaggcactcgccttgatggttcttactacacttggggcaagtggggtaagtcttggtgcttgaaacacttccctaagacttagattctggtgccctaaccttctgatcatacatgttcttagaggatggaatactagttatcatagttctgctatggggttgaaaacttttcctgactctacgagcgatttccaccamcaaatttctgctaagagtagtcatagttcccagtcctaacctcttattctccttggcttgtttcctaagcttatcaccctcaacctgctgatcatgagtcataagcttaaggatgttcatatctcgtagtaacatagaacttctgcactcagttttcaccaaattttgactccgtacaagaaattattcatctgagccct

SEQ ID NO: 105 - DNA, 401 bp, Lycopersicon esculentum
  Polymorphism (201)..(201): C=susceptible allele ; A=resistant allele
agtacaaattgcaactttttcaatgttaacgtctgcttttttagtgtataaaaaagaaggattcatgaactcctcagtcatattctattttaaaatacttaaacatcttttttaattatgtctcaagcatgtcaaacatctaatgcaaaaaagacttacgtttgtcgttgtggtaatttagctattttgaggatttcacamaccgatacgaacccagatcgataattatttaattgtgcaattggtgcattgatctttttttaagtggcttgattatgattcatcaacgagcaacccatcaaaattgagtctcggattcgagacatcaaattttcaaagcgttatcaaatttcaaatattctttatctccttattttatctcatgtacaacttgattgact

SEQ ID NO: 106 - DNA, 401 bp, Lycopersicon esculentum
  Polymorphism (201)..(201): C=susceptible allele ; A=resistant allele
tcatcgtagtcagtaagattttaccaaggaattaaaatttattaagtattaagctcattacattagaattagcaccaagatttccaaaaagaactaaattggaattagcaaaaatctgaaatttgaagagttaagttaagtatgagttttgagtcaacttcaaaggaccataaatattagaacacgatgagttagatgtgmtacaagataccatagggaagatattcgaataacctttgcaaagcctttgagtttactaagtttcaagttcatacaagtgagatatgaccttttgaagttaggttctccagttaagcaaagttagccaaaaatagtgaaggtattttggtccttcccttacccaaacagatttaattcgttttagtcatattttagggtta

SEQ ID NO: 107 - DNA, 401 bp, Lycopersicon esculentum
  Polymorphism (201)..(201): C=susceptible allele ; G=resistant allele
aaaacaatttcaattatgtagaatcatttttcggccacattaaatcatatttttatgtcattgaaccttgagaaaatactcatccatatcaattattaaaagtttaatttatctatacgctatatttcaattaacaataacgacatgcacgaattaaatttttaacaaatgacataaatgtgtcttgtctcaaaattcgasgagatctttcgttttctttaatattcatgaaaaaacattgatccaaagtggaaagttaaggatgagttggacatttttcacttaaaataatgtaacgtgactccctccattttccccaaacgtaagtccaagtcaacttgagacaccatattattataataaaaatagaacaaacacaaatccataaatttacaacttga

SEQ ID NO: 108 - DNA, 401 bp, Lycopersicon esculentum
  Polymorphism (201)..(201): G=susceptible allele ; A=resistant allele
ttccaatgggagaaacttgagaggaagtgaaaatgtggtggagataaacatggttatggagagacatttgaggcaaaatatgagagaaatatttgagagattttaggggattaaataagtgagagagaatattaattattaattattaataaagaaatgccaaatttatttacacaaaacgtgtaaaatggacattttgtrtcaatcttttaaaatctggctaatggatgctataaattgaagtctgagttgacatgctatgtcattttcctcaatgggctgttggagaagagaattgggttcaatatgggagacaaaacttggctagaattgagttaattactaagtttggttaaaaattaaataagataaataaattaatgttacttaattatgggaaa

SEQ ID NO: 109 - DNA, 401 bp, Lycopersicon esculentum
  Polymorphism (201)..(201): C=susceptible allele ; A=resistant allele
cgtggacggtactcgacgaccattgttggtccctaagcgaacccttatcctggctgactacaagcggaagactaacttaagcataccaaaaatcttaaaactgaaatgaacaacttagaataaaataatctatttaactagtcattaaataggcaacttgagtctttaaaacatttaataaaataaattattaataaaaamttctcaactagactatctatggagcctctaactgaaaaagatggaaatcgggacaccacgacatcttgattaaactgaaaagtaaataaggtcctccggaaacaaggaggttcaacgactaactcgaactctaggatgtattaataaagctcttgttaatgatcccgaagacctgtgtttgcatcatcaattatgcaacc

SEQ ID NO: 110 - DNA, 401 bp, Lycopersicon esculentum
  Polymorphism (201)..(201): G=susceptible allele ; A=resistant allele
agcttccttacttatttaaatgtgaaaacatatcatttggggatacttagttcccctatacttttgaagaaaagaacttgaacttaactctttactctttacttagcttgaaacttgactcttaaggaatatttagttcccttatattttttgagaaatgcactcactctttgctctttgctctatttgaaacttaactcrtagggaatacttaattcccttataaattttgagttttaaactcaaatcattactcttttctcaacttgtagtttaagccttaaaacaaagtttaaaatgttgtttaagactctttaaaatagtaagaacttactttgactttctttttatttgtaagacttgattcttaaattctttggctttgatattaactttccttg

SEQ ID NO: 111 - DNA, 401 bp, Lycopersicon esculentum
  Polymorphism (201)..(201): A=susceptible allele ; T=resistant allele
tatatcagaatgatcaaataatcgcgtctttagaattttataggcatcattgttgctgctaagatattttgacctaatgatcaaataatcgcgtctttagaattttataggcatcattgttgctgctaagatattttgataggtcaaatgatgtaatagggtttttgtagaggccagatcgtaaattggtgaaggcaaaawcagaaactgtacaatatgattttgatagagatcagttctttttcttgagatgttatggaggattcatctattaggatctagtacaaggaaaatttgttatgtattgaagtatttgttgagaacatttttgaattaccttgtgtaagtttaagattatgaagaattcttttctttttctttctactagatgggtaagcacg

SEQ ID NO: 112 - DNA, 301 bp, Lycopersicon esculentum
  allele (151)..(151): susceptible allele =  C; resistant allele = CT
agtgatattgtcttcagctatagaatattatccagcccctctagctttggtcgatttttttttataacaaattttatccaagctatcattgtttttttttttttttcaatttacagcaattgtttcccgactagcctatcaatgttagagcttttttttggtttgaacatcgttgtcttgagaagcttaatccatgttcactgtaggcattgttatttaattttcaattgagtttgttaatctgcctatagatattaatttatttcctcttctttcgatgcatatgatcaatccagtctat

SEQ ID NO: 113 - DNA, 301 bp, Lycopersicon esculentum
  allele (151)..(151): susceptible allele =  A;  resistant allele = AT
acaatgacatgacatcaaaaataatttaatttatcatgtcaaatttgtttaaaaataaattgtaatcgagtgattttaaagaataaagatgttaagttgagtgattttaaagacacaaactaaaatttagtgactttacaagataatttcatatagttcattgaccttttaagatattaactcaaaaaatcaataagtagtcacacacaaaaggtgattgcaaagtaaatttcaatttttagcataagcttagtggggaataccaatgacgaatctaaaagtatcaatacaaaaaatagaa

SEQ ID NO: 114 - DNA, 301 bp, Lycopersicon esculentum
  polymorphism (151)..(151): T=susceptible allele ; C=resistant allele
aaatgttaatttttttatttataataaatattaatatgtcttagacttgaacttttaagttcattaaagatagttaaatatcgtatttgatatacttaacgaaaaagctaaaacgtaaaagaaaatataataagtttatatcttcttcttytttttttaaatttggatttgtgatagtagggctttgagagaaaattctttttataattttttttttcatattcaaggctcgaacctaagatctatgattaaggaagaagtagcctttccactatacaatacacgttcataatgagttaat

SEQ ID NO: 115 - DNA, 301 bp, Lycopersicon esculentum
  allele (151)..(151): susceptible allele =  G;  resistant allele = GT
gcaaaaaaataaaaaataaaaaaacgctagacagattgatgactatttatgctcatttcaaaacataactaatttgtatttaatttttttttaccttttttgattattttttttgctgtatgattttctcggttctattattagttatttgtttttttaaatattaatcataataacataaatacataattaatcccttaacaaaataaaataatgacatttttaataaacaataaaattgttgctaagaagtcccactaaaagcttaaatattgttgttttgaaaaaaatttccccaagg

(19) United States
(12) Patent Application Publication
(10) Pub. No.: US 2023/0276763 A1
(43) Pub. Date: Sep. 7, 2023

(54) RESISTANCE IN PLANTS OF SOLANUM LYCOPERSICUM TO THE TOBRFV

(71) Applicant: VILMORIN & CIE, Paris (FR)

(72) Inventors: Lilian FONTANET, Saint Rémy de Provence (FR); Jeff SKONECZKA, Davis, CA (US); Eric LIONNETON, La Bohalle (FR); Julie LEDERER, Saint Rémy de Provence (FR)

(73) Assignee: VILMORIN & CIE, Paris (FR)

(21) Appl. No.: 18/008,372

(22) PCT Filed: Jun. 4, 2021
(86) PCT No.: PCT/EP2021/065076

§ 371 Date: Dec. 5, 2022

(30) Foreign Application Priority Data
Jun. 5, 2020 (WO) PCT/IB2020/000546

(51) Int. Cl. A01H 6/82; C12Q 1/6895; A01H 1/04; A01H 5/08

(52) U.S. Cl. CPC A01H 6/825; C12Q 1/6895; A01H 1/04; A01H 5/08

(57) ABSTRACT

A Solanum lycopersicum plant including a QTL giving the plant improved resistance to Tomato Brown Rugose Fruit virus, compared to a plant without the QTL, and wherein the QTL is introgressed from S. pimpinellifolium and is to be found on chromosome 9, within the chromosomal region delimited by the SNP TO-0201220 (SEQ ID NO:1) and the SNP having SEQ ID NO:101 or on chromosome 11, within the chromosomal region delimited by markers having SEQ ID NO:102 and 115. The QTL may be chosen from those present in the genome of a plant of the seeds LVSTBRFVRES2 NCIMB accession number 43591. The QTL includes defined alleles of different SNPs on chromosome 9 or 11.

The present invention relates to resistance in plants of Solanum lycopersicum, also known as Lycopersicum esculentum, to the tobamovirus Tomato Brown Rugose Fruit virus (ToBRFV, previously abbreviated TBRFV). More specifically, the present invention relates to tomato plants and fruits comprising a genetic determinant that leads to resistance to the Tomato Brown Rugose Fruit virus. According to the invention, the resistance is provided by DNA sequences, or QTL, introgressed from S. pimpinellifolium into the genome of S. lycopersicum plants, either on chromosome 9 or 11. The introgressed QTL on chromosome 9 can be present homozygously or heterozygously in the genome of a S. lycopersicum plant. The introgressed QTL on chromosome 11 is preferably present homozygously in the genome of a S. lycopersicum plant. The invention further relates to markers linked to said DNA sequences and to the use of such markers to identify or select the DNA sequences or QTLs and to identify or select plants carrying such resistance. The invention also relates to the seeds and progeny of such plants and to propagation material for obtaining such plants, and to different uses of these plants.

### BACKGROUND OF THE INVENTION

All cultivated and commercial forms of tomato belong to a species most frequently referred to as Lycopersicon esculentum Miller. Lycopersicon is a relatively small genus within the extremely large and diverse family Solanaceae which is considered to consist of around 90 genera, including pepper, tobacco and eggplant. The genus Lycopersicon has been divided into two subgenera, the esculentum complex which contains those species that can easily be crossed with the commercial tomato and the peruvianum complex which contains those species which are crossed with considerable difficulty (Stevens, M., and Rick, C. M. 1986). Due to its value as a crop, L. esculentum Miller has become widely disseminated all over the world. Even if the precise origin of the cultivated tomato is still somewhat unclear, it seems to come from the Americas, being native to Ecuador, Peru and the Galapagos Island and initially cultivated by Aztecs and Incas as early as 700 AD. Mexico appears to have been the site of domestication and the source of the earliest introduction. It is supposed that the cherry tomato, L. esculentum var. cerasiforme, is the direct ancestor of modern cultivated forms.

Tomato is grown for its fruit, widely used as a fresh market or processed product. As a crop, tomato is grown commercially wherever environmental conditions permit the production of an economically viable yield. The majority of fresh market tomatoes are harvested by hand at vine ripe and mature green stage of ripeness. Fresh market tomatoes are available year round. Processing tomato are mostly mechanically harvested and used in many forms, as canned tomatoes, tomato juice, tomato sauce, puree, paste or even catsup.

Tomato is a normally simple diploid species with twelve pairs of differentiated chromosomes. However, polyploidy tomato is also part of the present invention. The cultivated tomato is self-fertile and almost exclusively self-pollinating. The tomato flowers are hermaphrodites. Commercial cultivars were initially open pollinated. As hybrid vigor has been identified in tomatoes, hybrids are replacing the open pollinated varieties by gaining more and more popularity amongst farmers with better yield and uniformity of plant characteristics. Due to its wide dissemination and high value, tomato has been intensively bred. This explains why such a wide array of tomato is now available. The shape may range from small to large, and there are cherry, plum, pear, blocky, round, and beefsteak types. Tomatoes may be grouped by the amount of time it takes for the plants to mature fruit for harvest and, in general the cultivars are considered to be early, midseason or late-maturing. Tomatoes can also be grouped by the plant's growth habit; determinate, semi-determinate or indeterminate. Determinate plants tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties. More recent developments in tomato breeding have led to a wider array of fruit color. In addition to the standard red ripe color, tomatoes can be creamy white, lime green, pink, yellow, golden, orange or purple.

Hybrid commercial tomato seed can be produced by hand pollination. Pollen of the male parent is harvested and manually applied to the stigmatic surface of the female inbred. Prior to and after hand pollination, flowers are covered so that insects do not bring foreign pollen and create a mix or impurity. Flowers are tagged to identify pollinated fruit from which seed will be harvested.

A variety of pathogens affect the productivity of tomato plants, including virus, fungi, bacteria, nematodes and insects. Tomatoes are inter alia susceptible to many viruses and virus resistance is therefore of major agricultural importance.

Tobamoviruses are among the most important plant viruses causing severe damages in agriculture, especially to vegetable and ornamental crops around the world. Tobamoviruses are easily transmitted by mechanical means, as well as through seed transmission. Tobamoviruses are generally characterized by a rod-shaped particle of about 300 nm encapsidating a single stranded, positive RNA genome encoding four proteins. In tomatoes, tobacco mosaic virus (TMV) and tomato mosaic virus (ToMV) are feared by growers worldwide as they can severely damage crop production, for example through irregular ripening (fruits having yellowish patches on the surface and brownish spots beneath the surface). Several genes have however been identified by plants breeders over the years and TMV and/or ToMV resistant tomato varieties are nowadays available.

For the last decades, all modern indeterminate tomato varieties and many of the determinate tomato varieties indeed contain the Tm-2 gene or preferably the Tm-2² allele of this gene, which give them immunity to almost all known races of Tobamoviruses which affected commercial tomatoes (ToMV and TMV) before 2014.

During 2014-2015, a severe outbreak of virus affected tomato productions areas in the middle east, such as in Jordan and in Israel. Most of the tomato varieties affected were considered TMV and/or ToMV resistant, but were still severely affected and showed typical TMV/ToMV like symptoms: while the foliar ones were quite similar to the TMV/ToMV symptoms, the fruit symptoms were much more frequent and severe than the usual symptoms from such viruses with fruits lesions and deformations. The fruit quality was very poor and rather unmarketable. Salem et al (Arch. Virol. 161 (2), 503-506. 2015) extracted RNA from fruit and leaves of symptomatic plants, infected in Jordan, and made various tests leading to the identification of a new Tobamovirus species, the sequence of which corresponds to GenBank accession no. KT383474 (SEQ ID NO:112); Salem et al proposed to name this Jordanian virus: Tomato Brown Rugose Fruit virus (previously TBRFV and now ToBRFV). The comparison to other Tobamoviruses sequences showed that it is indeed a Tobamovirus, but not TMV or ToMV. The resistance to TMV and/or ToMV does not confer resistance to this new virus ToBRFV. Luria et al (PLoS One. 2017; 12(1): e0170429) have concomitantly isolated and sequenced the complete genome of the Israeli tobamovirus infecting tomato in Israel, corresponding to GenBank accession no. KX619418 (SEQ ID NO:113). They have thus shown a very high sequence identity between the Israeli and the Jordanian viruses (more than 99% sequence identity) and have concluded to two different isolates of tomato brown rugose fruit virus.

Recently, the virus was identified in Europe, especially in Sicily, Germany, the Netherlands and France, and in Mexico, and therefore now it is considered as a major global threat to tomato crop. The strain identified appears to be essentially the Israeli strain, rather than the Jordanian strain.

Identification of tomato plants which display resistance to the Tomato Brown Rugose Fruit virus and localization and identification of genetic determinants, also referred to hereafter as QTLs (Quantitative Trait Locus) that lead to resistance to the Tomato Brown Rugose Fruit virus have recently been described in WO2018/219941, although reference is made to tolerance in this publication. Two QTLs, namely QTL1 and QTL2, are to be found on chromosome 6 and 9 respectively, and confer independently or in combination an improved tolerance or resistance in the fruits of a tomato plant infected or likely to be infected by the ToBRFV, when present homozygously into a S. lycopersicum background. A third QTL, QTL3, is to be found on chromosome 11, and confers an improved tolerance or resistance in the leaves of a tomato plant infected or likely to be infected by the ToBRFV, when present homozygously.

Whereas these QTLs, either alone or in combination, provide tolerance or resistance to ToBRFV, the inventors have now established that, most of the time, they cannot confer a sufficiently high level of resistance to the tomato plants, such that a significant part of the fruits are affected and are not marketable. Moreover, these QTLs are described as providing resistance when present homozygously. Insofar as QTL2, on chromosome 9, is present at the same locus as the Tm-2² gene, in a region which is generally transmitted “en bloc” without recombination, such a QTL on chromosome 9 is therefore not suitable for combination with the Tm-2² gene.

WO2019/110130 and WO2019/110821 disclose the identification of 3 different QTLs on chromosomes 6, 11 and 12, introgressed from S. pimpinellifolium and allegedly conferring resistance or tolerance to ToBRFV. The QTL on chromosome 11 is however described as located between 2 markers which define a region of 55 Mbases, corresponding to almost the whole sequence of chromosome 11. Without any clearer description, this QTL on chromosome 11 cannot be used for introgression.

WO2020/018783 discloses a genetic region on tomato chromosome 11 that comprises a Stemphylium resistance allele from S. pimpinellifolium, and allegedly also comprises an associated ToBRFV resistance allele. As both alleles are linked to the same markers according to the disclosure of this document, introgression of only ToBRFV resistance is not made possible, especially in plants already resistant to Stemphylium but susceptible to ToBRFV.

As Tobamoviruses are not easily controlled but through genetic improvement by the identification and use in breeding of resistance genes, and as the resistance genes currently available to control TMV and/or ToMV are useless against the damages from the new Tomato Brown Rugose Fruit virus, and the tolerance or resistance QTLs already identified are not always sufficiently efficient, not sufficiently characterized and not combinable with Tm-2², there is an urgent need to identify resistance against this new Tobamovirus, failing that would result in entire regions in which tomato crop could not be produced anymore.

### SUMMARY

The present inventors have identified a resistance against ToBRFV in a wild S. pimpinellifolium plant and have been able to introgress this resistance into S. lycopersicum plants, thus obtaining resistant S. lycopersicum tomato plants to ToBRFV. The resistance of the present invention is imparted by the newly discovered sequences, linked to additive quantitative trait loci (QTL), transferable to different S. lycopersicum genetic backgrounds.

The newly discovered QTLs confer a resistance to the Tomato Brown Rugose Fruit virus (ToBRFV) essentially at the level of the fruits of the tomato plants infected by the virus, and also at the level of the leaves of the tomato plants infected by the virus, for the QTL on chromosome 9, and essentially at the level of the leaves of the infected plants for the QTL on chromosome 11.

The present invention thus provides these introgressed sequences, also here named QTLs, conferring the phenotype of ToBRFV resistance at the level of the tomato leaves and/or fruits of the tomato plants infected by the ToBRFV.

The present invention provides S. lycopersicum plants that display resistance to ToBRFV, including commercial plants, lines and hybrids, as well as methods that produce or identify S. lycopersicum plants or populations (germplasm) that display resistance to ToBRFV. The present invention also discloses molecular genetic markers, especially Single Nucleotide Polymorphisms (SNPs), linked to the QTLs of the invention responsible for resistance to the ToBRFV. Plants obtained through the methods and uses of such molecular markers are also provided.

Said resistance is moreover easily transferable to different genetic backgrounds, i.e. into various tomatoes, and the invention also extends to different methods allowing the transfer or introgression of the QTL conferring the phenotype.

The invention also provides several methods and uses of the information linked to these SNPs associated to the QTL conferring the ToBRFV resistance, inter alia methods for identifying ToBRFV resistant plants and methods for identifying further molecular markers linked to this resistance, as well as methods for improving the yield of tomato production in an environment infested by ToBRFV and methods for protecting a tomato field from ToBRFV infestation.

### Definitions

The term “Resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure. Two levels of resistance are defined:

High Resistance (HR): plants that highly restrict the growth and/or development of the specified pest and/or the damage it causes under normal pest pressure when compared to susceptible plants. These plants may, however, exhibit some symptoms or damage under heavy pest pressure.

Intermediate Resistance (IR): plants that highly restrict the growth and/or development of the specified pest and/or the damage it causes but may exhibit a greater range of symptoms or damage compared to high resistance plants. Intermediate resistant plants will still show less severe symptoms or damage than susceptible plants when grown under similar environmental conditions and/or pest pressure.

The term “Tolerance” is normally used to describe the ability of a plant to endure abiotic stresses without serious consequences for growth, appearance and yield.

In the literature and patents, this term is however also used to indicate a phenotype of a plant wherein at least some of the disease-symptoms remain absent upon exposure of said plant to an infective dose of virus, whereby the presence of a systemic or local infection, virus multiplication, at least the presence of viral genomic sequences in cells of said plant and/or genomic integration thereof can be established, at least under some culture conditions. Tolerant plants are therefore resistant for symptom expression but symptomless carriers of the virus. Sometimes, viral sequences may be present or even multiply in plants without causing disease symptoms. It is to be understood that a tolerant plant, although it is infected by the virus, is generally able to restrict at least moderately the growth and development of the virus.

For this reason, tolerant plants according to this definition are best characterized by Intermediate Resistant plants.

In case of ToBRFV, by leave resistance, or foliar resistance, it is meant the phenotype of a plant wherein the disease symptoms on the leaves remain absent, or are less important, upon exposure of said plant to an infective dose of ToBRFV. Disease symptoms on the fruits may however be present on infected plants.

By fruit resistance, in case of ToBRFV, it is meant the phenotype of a plant wherein the disease symptoms on the fruits remain absent, or are less important, upon exposure of said plant to an infective dose of ToBRFV. Disease symptoms on the leaves may however be present on infected plants.

Symptoms on leaves of ToBRFV infection generally include mosaic, distortion of the leaflets and in many cases also shoestrings like symptoms. Symptoms on fruits of ToBRFV infection generally include typical yellow lesions (discoloration) and deformation of the fruits. In many cases there are also “chocolate spots” on the fruits.

Susceptibility: The inability of a plant to restrict the growth and development of a specified pest or pathogen; a susceptible plant displays the detrimental symptoms linked to the virus infection, namely the foliar damages and fruit damages in case of ToBRFV infection.

A S. lycopersicum plant susceptible to Tomato Brown Rugose Fruit virus, is for example the commercially available variety Candela as mentioned in the 2015 Salem et al. publication.

All commercially available varieties of tomato grown in ToBRFV infected area are, to date, i.e. before the present invention, susceptible to ToBRFV, or not sufficiently resistant for those plants bearing the tolerance QTLs, such as the deposited seeds of HAZTBRFVRES1, mentioned in the PCT application WO2018/219941.

A plant according to the invention has thus at least improved resistance or tolerance to ToBRFV, with respect to the variety Candela, and more generally with respect to any commercial variety of tomato grown in ToBRFV infected area, including tolerant plants, and with respect to HAZTBRFVRES1. The improved resistance with respect to the plants corresponding to HAZTBRFVRES1 is demonstrated in example 5 of the experimental section.

As used herein, the term “offspring” or “progeny” refers to any plant resulting as progeny from a vegetative or sexual reproduction from one or more parent plants or descendants thereof. For instance, an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “genetic determinant” and/or “QTL” refers to any segment of DNA associated with a biological function. Thus, QTLs and/or genetic determinants include, but are not limited to, genes, coding sequences and/or the regulatory sequences required for their expression. QTLs and/or genetic determinants can also include nonexpressed DNA segments that, for example, form recognition sequences for other proteins.

As used herein, the term “genotype” refers to the genetic makeup of an individual cell, cell culture, tissue, organism (e.g., a plant), or group of organisms.

As used herein, the term “grafting” is the operation by which a rootstock is grafted with a scion. The primary motive for grafting is to avoid damages by soil-born pest and pathogens when genetic or chemical approaches for disease management are not available. Grafting a susceptible scion onto a resistant rootstock can provide a resistant cultivar without the need to breed the resistance into the cultivar. In addition, grafting may enhance tolerance to abiotic stress, increase yield and result in more efficient water and nutrient uses.

As used herein, the term “heterozygote” refers to a diploid or polyploid individual cell or plant having different alleles (forms of a given gene, genetic determinant or sequences) present at least at one locus.

As used herein, the term “heterozygous” refers to the presence of different alleles (forms of a given gene, genetic determinant or sequences) at a particular locus.

As used herein, “homologous chromosomes”, or “homologs” (or homologues), refer to a set of one maternal and one paternal chromosomes that pair up with each other during meiosis. These copies have the same type of genes at the same loci and the same centromere location, but they may differ by their sequences or alleles.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “locus” (plural: “loci”) refers to any site that has been defined genetically, this can be a single position (nucleotide) or a chromosomal region. A locus may be a gene, a genetic determinant, a part of a gene, or a DNA sequence, and may be occupied by different sequences. A locus may also be defined by a SNP (Single Nucleotide Polymorphism), by several SNPs, or by two flanking SNPs.

As used herein, the term “rootstock” is the lower part of a plant capable of receiving a scion in a grafting process.

As used herein, the term “scion” is the higher part of a plant capable of being grafted onto a rootstock in a grafting process.

The invention encompasses plants of different ploidy levels, essentially diploid plants, but also triploid plants, tetraploid plants, etc.

In the context of the present invention, DNA strand and allele are designed TOP according to the TOP/BOT designation method developed by Illumina: (https://www.illumina.com/documents/products/technotes/technote_topbot.pdf).

### DETAILED DESCRIPTION OF THE INVENTION

The present inventors have identified QTLs which, when present in a S. lycopersicum plant, alone or in combination, provide an improved resistance in the fruits and/or leaves of a tomato plant infected or likely to be infected by the Tomato Brown Rugose Fruit virus (ToBRFV).

The present inventors have identified one major QTL on chromosome 9, referred to as QTL9 in the following, which confers resistance to ToBRFV infection, especially fruit resistance, when present in a S. lycopersicum background, and one additional QTL on chromosome 11, referred to as QTL11 in the following, which also confers resistance to ToBRFV, especially leaf resistance, and can be combined with the QTL9 as defined above.

The seeds and plants according to the invention have been obtained from an initial cross between a wild plant of S. pimpinellifolium, the introgression partner displaying the phenotype of interest but in another species, and a plant of S. lycopersicum, the recurrent susceptible parent, in order to transfer the resistance into S. lycopersicum genetic background.

Seeds of resistant S. lycopersicum plants, deriving from this initial cross, which comprise homozygously QTL9 and QTL11, and called LVSTBRFVRES2, have been deposited by the inventors at the NCIMB under the accession number NCIMB 43591 on 1 Apr. 2020. The plants grown from these deposited seeds are S. lycopersicum tomatoes, resistant against ToBRFV, namely displaying at least an improved fruit resistance to this virus with respect to any known S. lycopersicum plants, especially any commercial S. lycopersicum plants.

As demonstrated in the examples, the phenotype of the plants according to the invention is best characterized as resistance rather than tolerance to ToBRFV, namely fruit resistance or both foliar and fruit resistance, tolerance being applicable only to abiotic stresses. Insofar as tolerance has also been widely used also for characterizing resistance or intermediate resistance, the plants of the invention however may also be characterized as tolerant plants. In the following, reference is made to resistance to ToBRFV; this phenotype however encompasses tolerance phenotype, as defined in some publications, as well as intermediate resistance.

Moreover, also as demonstrated in the examples, inter alia examples 4, 5 and 8, the ToBRFV resistance according to the invention is distinct from the tolerance/resistance disclosed in the prior art. Indeed, example 5 demonstrates that the level of resistance according to the invention is higher than the level of resistance described in WO2018/219941. Moreover, example 4 confirms that the sequences responsible for the ToBRFV resistance are different according to the present invention and according to WO2018/219941. Example 8 demonstrates that the ToBRFV resistance according to the invention is not associated to Stemphylium resistance, contrary to the genetic determinant disclosed in WO2020/018783, and is thus distinct from this resistance.

According to a first aspect, the invention is thus directed to a S. lycopersicum plant comprising in its genome a QTL on chromosome 9, hereinafter referred to as QTL9, and/or a QTL on chromosome 11, hereinafter referred to as QTL11, conferring said improved resistance to ToBRFV in case of infection, especially at the fruit level for QTL9 and at the foliar level for QTL11, as well as seeds and cells of such a tomato plant, comprising the QTL9 and/or the QTL11 in their genome.

Said QTLs conferring the resistance were initially introgressed from a wild S. pimpinellifolium, and are thus referred to as the resistance QTLs, or QTL9 or QTL11, or introgressed sequences of the invention in the following description. The invention is also directed to a cell of such a plant or seed, comprising these introgressed sequences conferring the resistance.

The tolerance/resistance phenotype can be tested and scored as described in the experimental section, especially in example 1, by natural infection or by artificial inoculation, at the first leaves level, or at the fruit level.

The QTL conferring the improved resistance to ToBRFV is preferably located on chromosome 9, within a chromosomal interval or region delimited by the SNP TO-0201220 (SEQ ID NO.1) and the SNP having SEQ ID NO.101.

The inventors have indeed demonstrated in the example section that introgressed sequences in this region, corresponding to QTL9, are inherited with the phenotype of interest.

The QTL9 according to the invention and conferring the improved resistance to ToBRFV is chosen from the ones present in the genome of seeds of LVSTBRFVRES2. The QTL9 is thus present in the genome of these deposited seeds. A sample of these S. lycopersicum seeds has been deposited by HM.Clause S.A., rue Louis Saillant, 26800 Portes-les-Valence, France, pursuant to and in satisfaction of the requirements of the Budapest treaty on the International Recognition of the deposit of Microorganisms for the Purpose of Patent procedure (“the Budapest Treaty” with the National collection of Industrial, Food and Marine bacteria (NCIMB) (NCIMB, Ltd, Ferguson Building, Craibstone Estate, Bucksburn, Aberdeen AB21 9YA, united Kingdom) on 1 Apr. 2020 under accession number 43591. A deposit of this tomato seed is maintained by HM.Clause S.A., rue Louis Saillant, 26800 Portes-les-Valence, France.

The other QTL according to the invention conferring the improved resistance to ToBRFV is preferably located on chromosome 11, within a chromosomal interval or region delimited by SNP TO-0201237 (SEQ ID NO:102) and SL2.50ch11_9924232 (SEQ ID NO:115), preferably by SNP TO-0201237 (SEQ ID NO:102) and SNP TO-0201241 (SEQ ID NO:106). The QTL11 according to the invention and conferring the improved resistance to ToBRFV, which can be in combination with QTL9, is chosen from the ones present in the genome of seeds of LVSTBRFVRES2, NCIMB 43591. The QTL11 is indeed present in the genome of the seeds of LVSTBRFVRES2 NCIMB accession number 43591.

The specific polymorphisms corresponding to the SNPs (Single Nucleotide Polymorphism) or markers referred to in this description, as well as the flanking sequences of these SNPs or markers in the S. lycopersicum genome, are given in the experimental section (see inter alia tables G and H for QTL9 and table K for QTL11) and the accompanying sequence listing. Their location with respect to the version 2.50 of the tomato genome, on chromosomes 9 and 11, and their flanking sequences are also illustrated.

It is to be noted in this respect that, by definition, a SNP refers to a single nucleotide in the genome, which is variable depending on the allele which is present, whereas the flanking nucleotides are identical. For ease of clear identification of the position of the different SNPs, their position is given in tables G, H and K, by reference to the tomato genome sequence in its version 2.50 and by reference to their flanking sequences, identified by SEQ ID number. In the sequence associated with a specific SNP in the present application, for example SEQ ID NO:1 for the SNP TO-0201220, only one nucleotide within the sequence actually corresponds to the polymorphism, namely the 201^{st} nucleotide of SEQ ID NO:1 corresponds to the polymorphic position of SNP TO-0201220, which can be A or G as indicated in table G. The flanking sequences are given for positioning the SNP in the genome but are not part of the polymorphism as such. Detection of a SNP marker, or of an allele of this SNP therefore refers to the detection of the polymorphic nucleotide of this marker, and does not require all the flanking sequences to be identical.

Similarly, the other markers referred to in the description and in Table K, which are not strictly speaking SNP, are INDEL markers, marking the insertion of a single nucleotide. For example, for marker SL2.50ch11_9684449 (SEQ ID NO:112) at position 9684449 of SL2.50 according to table K, this means that the position 9684449 is allelic, i.e. is either C or CT, corresponding to insertion of a T. the position of the “C” in SL2.50 is the position 9684449 mentioned in table K. Insofar as the allele concerns only one position for these INDEL markers, for the sake of simplicity, they can also be referred to as SNP marker in the following, by linguistic extension.

A genomic or chromosomal region identified by flanking sequences, e.g. SNPs or INDEL markers (assimilated to SNPs in the following), is thus defined clearly and non-ambiguously.

A genomic region delimited by two SNPs X and Y refers to the section of the genome, more specifically of a chromosome, lying between the positions of these two SNPs and preferably comprising said SNPs, therefore the nucleotide sequence of this chromosomal region begins with the nucleotide corresponding to SNP X and ends with the nucleotide corresponding to SNP Y, i.e. the SNPs are comprised within the region they delimit, according to the invention.

By “introgressed sequences from S. pimpinellifolium” in a given genomic region, it is to be understood that the genomic sequences found in this region have the same sequence as the corresponding genomic sequences found in the S. pimpinellifolium donor, i.e. in the introgression partner, at the same locus and also the same sequence as the corresponding genomic sequences found in LVSTBRFVRES2 (NCIMB 43591) at the same locus. By having the “same sequence”, it means that the two sequences to be compared are identical to the exception of potential point mutations which may occur during transmission of the genomic region to progeny, i.e. preferably at least 99% identical on a length of 1 kbase.

It can be concluded that a given genomic region has the same sequence, in the sense of the invention, as the corresponding genomic region found in the S. pimpinellifolium donor at the same locus, if said genomic region is also capable of conferring resistance to ToBRFV and is of S. pimpinellifolium origin.

The presence of introgressed sequences into the genome of a S. lycopersicum plant, seed or cell may for example be shown by GISH (genetic in situ hybridization). GISH is indeed a powerful technique for detection of the introgression of chromatin material from one species or subspecies onto another species. The advantage of GISH is that the introgression process is visualized by means of ‘pictures of the introgressed genome’. With this technique, it is also possible to establish if a particular region of the genome is homozygous or heterozygous, thanks to the use of molecular cytogenetic markers which are co-dominant. By this technique, it is also possible to determine in which chromosome an introgressed gene of interest is present.

The present inventors have identified and mapped the QTLs imparting the ToBRFV resistance of the invention, mainly by identifying the presence of sequences representative of the introgressed QTLs at different loci along the region of chromosomes 9 and 11 mentioned above, namely at 101 different loci defined by the 101 SNPs having SEQ ID NO.1 to 101 for QTL9 and at 14 different loci defined by 14 markers having SEQ ID NO:102 to 115 for QTL11. These SNPs are referred to in the following as the SNPs of the invention, or the 101 SNPs of the invention for QTL9. Preferred SNPs amongst them are the 14 SNPs having SEQ ID NO:1 to 14; especially SNPs having SEQ ID NO:1, 2, 10, 12 and 14.

The presence of the introgressed sequences, or QTL, conferring the resistance phenotype can thus be identified on the basis of these SNP markers, in the genome of a plant, seed or cell of the invention.

Preferably, for QTL9, the presence of the introgressed sequences, or QTL, is thus identified or characterized in a tomato plant by one of the 101 SNPs having SEQ ID NO:1-101 and preferably by a SNP chosen from the list of 14 SNPs comprising SNP TO-0201220 (SEQ ID NO:1), TO-0201221 (SEQ ID NO:2), TO-0201222 (SEQ ID NO:3), TO-0201223 (SEQ ID NO:4), TO-0201224 (SEQ ID NO:5), TO-0201225 (SEQ ID NO:6), TO-0201226 (SEQ ID NO:7), TO-0201227 (SEQ ID NO:8), TO-0201228 (SEQ ID NO:9), TO-0201229 (SEQ ID NO:10), TO-0201230 (SEQ ID NO:11), TO-0201231 (SEQ ID NO:12), TO-0201232 (SEQ ID NO: 13) and TO-0201233 (SEQ ID NO:14), and more preferably by one of the 5 SNPs TO-0201220, TO-0201221, TO-0201229, TO-0201231 and TO-0201233. The QTL9 of the invention may for example be identified or characterized by the SNP TO-0201220 or by the SNP TO-0201229.

Another suitable SNPs are those having SEQ ID NO: 15, 16, 17, 18, 19, 20, 21, 22, 23, 24, 25, 26; 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 and 101. According to a preferred embodiment, for QTL9, the presence of the introgressed sequences in a tomato plant, cell or seed of the invention is identifiable by at least 2, preferably at least 3, or at least of said 101 SNP markers, or of said 14 SNP markers, preferably at least one of them is SNP TO-0201220 or SNP TO-0201229. For example, the presence of the introgressed sequences conferring ToBRFV resistance are detected by the presence of a haplotype constituted by at least 2 SNPs, for example one being TO-0201229 and the other one(s) being different SNP(s) chosen from the SNPs having SEQ ID NO:1-101 (except SEQ ID NO:10).

The alleles of these molecular markers, representative of the QTL or introgressed sequences conferring the resistance of the invention are reported in the last column of table H for the 101 SNPs of the invention. For the 14 SNPs markers mentioned above, the alleles representative of the introgressed QTL are allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-020132 and allele G of TO-020133.

The presence of the QTL in the genome of a tomato plant, cell or seed according to the invention can thus be detected or revealed by detecting sequences representative of the QTL at said loci, more preferably by detecting one or more of the resistant alleles of the SNPs having SEQ ID NO:1-101, as reported in the last column of table H, for example by detecting at least one of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-0201233, more preferably by detecting allele G of SNP TO-0201220, allele G of SEQ ID TO-0201221, allele A of TO-0201229, allele C of TO-0201231 or allele G of TO-0201233, and even more preferably by detecting allele G of SNP TO-0201220 and/or allele A of TO-0201229.

According to a preferred embodiment, the QTL of the invention on chromosome 9, QTL9, is detected in the genome of a tomato plant, cell or seed, by detecting at least 2, preferably 3, preferably at least of the resistant alleles of the SNP having SEQ ID NO:1 to 101, preferably at least one the detected resistant allele being allele G of SNP TO-0201220 and/or allele A of TO-0201229.

The QTL9 is on a chromosomal interval of chromosome 9 delimited or flanked on one side by the flanking SNP TO-0201220 and on the other side by the flanking SNP marker having SEQ ID NO.101. A more preferred chromosomal interval of chromosome 9 within which QTL9 is to be found is the interval delimited by TO-0201220 and TO-0201233. An even more preferred interval is the interval delimited by the SNP having SEQ ID NO:20 and TO-0201233, or the interval between the SNP having SEQ ID NO:22 and TO-0201233, or the interval between the SNP having SEQ ID NO:26 and TO-0201233, or the interval between the SNP having SEQ ID NO:30 and TO-0201233, or the interval between the SNP having SEQ ID NO:34 and TO-0201233, or the interval between the SNP having SEQ ID NO:38 and TO-0201233, or preferably the interval between SNPs TO-0201221 and TO-0201233.

The QTL11 according to the invention is preferably detected by one of the markers having SEQ ID NO:102 to 115, preferably by one of the SNP markers having SEQ ID NO:102 to 111, preferably by at least one of the SNPs TO-0201237 (SEQ ID NO:102), TO-0201238 (SEQ ID NO:103), TO-0201239 (SEQ ID NO:104), TO-0201240 (SEQ ID NO:105) and TO-0201241 (SEQ ID NO:106), and/or by at least one of the markers SL2.50ch11_9684449 (SEQ ID NO:112), SL2.50ch11_9779896 (SEQ ID NO:113), SL2.50ch11_9823405 (SEQ ID NO:114) and SL2.50ch11_9924232 (SEQ ID NO:115). Preferably, the presence of this QTL11 can be characterized by the detection of at least one of the resistant alleles of the markers having SEQ ID NO:102 to 115, preferably by one of the resistant alleles of the SNPs having SEQ ID NO:102 to 111 and/or of the markers having SEQ ID NO:112 to 115, as disclosed in the last column of Table K. According to a preferred embodiment, the presence of the QTL11 is characterized by the detection of at least one of allele CT of SL2.50ch11_9684449, allele AT of SL2.50ch11_9779896, allele C of SL2.50ch11_9823405, allele GT of SL2.50ch11_9924232, allele G of TO-0201237, allele A of TO-0201238, Allele A of TO-0201239, allele A of TO-0201240 and allele A of TO-0201241, preferably by at least one of allele G of TO-0201237, allele A of TO-0201238, Allele A of TO-0201239, allele A of TO-0201240 and allele A of TO-0201241 and/or at least one of allele CT of SL2.50ch11_9684449, allele AT of SL2.50ch11_9779896, allele C of SL2.50ch11_9823405 and allele GT of SL2.50ch11_9924232. Preferred markers for QTL11 are thus those having SEQ ID NO:102 to 115 (table K), alternatively those having SEQ ID 102-111, alternatively the list of markers TO-0201237, TO-0201238, TO-0201239, TO-0201240, TO-0201241, SL2.50ch11_9684449, SL2.50ch11_9779896, SL2.50ch11_9823405 and SL2.50ch11_9924232, alternatively the list of markers TO-0201237, TO-0201238, TO-0201239, TO-0201240 and TO-0201241, and alternatively the list of markers SL2.50ch11_9684449, SL2.50ch11_9779896, SL2.50ch11_9823405 and SL2.50ch11_9924232. The preferred resistant alleles are those corresponding to these different lists, and are given in Table K. These lists of markers and/or of resistant alleles are applicable to all the different aspects of the present invention.

A tomato S. lycopersicum plant, cell or seed of the invention may be homozygous for the QTL9, QTL11, or introgressed sequences of the invention conferring ToBRFV resistance. The invention is however not limited to such homozygous plants, cells or seeds. Indeed, the inventors have also demonstrated that the resistance imparted by this QTL9 is additive, such plants having heterozygously the QTL9 of the invention are also resistant to ToBRFV (see experimental section), at a level which is below the resistance level of the plants comprising the QTL homozygously, but above the level of susceptible plants. The present invention thus also encompasses tomato S. lycopersicum plant, cell or seed having heterozygously in their genome on chromosome 9 the QTL9, or introgressed sequences, of the invention as defined above.

QTL11 may also be present homozygously or heterozygously, in a S. lycopersicum plant, cell or seed of the invention, but only confers ToBRFV resistance at the homozygous stage, as the resistance allele is recessive (see example 9 in this respect). The heterozygous or homozygous presences of QTL9 and QTL11 can be defined independently.

According to a preferred embodiment, a plant, seed or cell of the invention comprises the QTL9, heterozygously or homozygously, as well as the QTL11 as defined above, either homozygously or heterozygously. Preferred combinations are QTL9 and QTL11 both homozygously present, QTL9 heterozygously with QTL11 homozygously, and both QTLs heterozygously present.

Preferably, a S. lycopersicum plant according to the invention is a commercial plant or line. Such a commercial plant or line preferably also exhibits resistance to ToMV (tomato mosaic virus), for example due to the presence of a Tm-2 gene (allele Tm-2 or Tm-2² (also known as Tm-2a)) which also confers resistance to TMV (Tobacco Mosaic Virus). A plant according to this aspect of the invention preferably has also the following additional features: nematode resistance trait (Mi-1 or Mi-j), as well as Fusarium and Verticillium resistances, and TYLCV resistance.

Other resistances or tolerances are also envisaged according to the invention.

According to a preferred embodiment, a plant of the invention is not resistant to Pepino Mosaic Virus (PepMV). According to another embodiment, a tomato plant of the invention is also resistant to PepMV.

Moreover, the commercial plant of the invention gives rise to fruits in suitable conditions, which are at least 10 grams, preferably 25 grams at full maturity, preferably at least 100 g at full maturity and or even more preferably at least 150 g or at least 200 g at full maturity. The number of fruits per plant is moreover essentially unaffected by the presence of the QTL9 of the invention, i.e. the productivity of a plant according to the invention is not inferior by more than 20% to a plant having the same genotype but devoid of said QTL9. A plant of the invention, for example a plant grown from the deposited seeds, thus generally bears at least 3, preferably around 4 tomatoes per cluster, and these fruits have preferably a weight between 150 g and 180 g.

According to still another embodiment, a plant of the invention is a determinate, indeterminate or semi-indeterminate plant, or seed or cell thereof, i.e. corresponding to determinate, indeterminate or semi-indeterminate growth habit.

By determinate, it is meant tomato plants which tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties.

The invention is also directed to hybrid plants of S. lycopersicum, obtainable by crossing a plant bearing homozygously the QTL9 of the invention, with another S. lycopersicum. As the QTL9 of the present invention is additive, the hybrid plants of S. lycopersicum produced by the above described cross will have resistance to ToBRFV. Preferably, the other S. lycopersicum crossing partner is devoid of said QTL9 of the present invention, but may comprise one of the QTLs described in WO2018/219941, preferably QTL2 on chromosome 9 or QTL3 on chromosome 11. Alternatively, the other S. lycopersicum crossing partner may comprise the QTLs described in WO2020/018783, WO2019/110130 and WO2019/110821, on chromosome 11, as imparting ToBRFV resistance.

The invention is thus also directed to tomato plant, seed or cell, comprising homozygously or heterozygously the QTL9 as disclosed, as well as:
The QTL2 on chromosome 9, as disclosed in WO2018/219941; heterozygously;
The QTL3 on chromosome 11, as disclosed in WO2018/219941, preferably homozygously;
One or more of the QTLs on chromosome 11, as disclosed in WO2020/018783, WO2019/110130 and WO2019/110821, homozygously or heterozygously.

Moreover, insofar as the inventors of the present invention have identified a QTL on chromosome 11, referred to as QTL11 in the following, which confers ToBRFV resistance, this QTL11 may be present in combination with the QTL9 of the invention. This QTL on chromosome 11, also corresponds to introgressed sequences from the S. pimpinellifolium introgression partner having provided the QTL9. The introgressed sequences corresponding to QTL11 are to be found on chromosome 11 within the region delimited by SNP TO-0201237 (SEQ ID NO:102) and SNP TO-0201241 (SEQ ID NO:106). As demonstrated in the experimental section of the application, this QTL11, when present in the genome of a tomato plant, especially in combination with the QTL9; provides an increased resistance to ToBRFV with respect to the same plant, devoid of said QTL11. Each QTL thus independently confers ToBRFV resistance, but the combination of the QTLs provides an increased resistance with respect to the resistance provided by only one QTL, i.e. is at least cumulative, especially as the resistance is essentially at the leaf level for QTL11 and at the fruit level for QTL9. The present invention is thus also directed to tomato plants, cells and seeds comprising in their genome this QTL11, either homozygously or heterozygously, but preferably homozygously in order to confer ToBRFV resistance, preferably leaf resistance. Preferably, this QTL11 is to be found in combination with the QTL9 of the invention.

The invention thus also concerns tomato plants comprising QTL9 and QTL11, thus increasing the resistance level to ToBRFV of the plant, with respect of a corresponding plant devoid of said QTL11; the invention also encompasses cells and seed thereof.

Moreover, as disclosed above, presence of a resistance gene providing ToMV and TMV resistance is advantageous, especially for indeterminate commercial varieties. According to some preferred embodiments of the invention, the Tm-2² gene (also known as Tm-2² or Tm-2(2)) conferring both ToMV and TMV resistances is thus combined with the QTL9 of the invention, with the QTL11 of the invention, or with QTL9 and QTL11. The inventors have however noted that the Tm-2² gene and QTL9 are positioned on the same arm of chromosome 9, in a region transmitted “en bloc”, not prone to recombination events. Combining the Tm-2² gene and the QTL9 on the same chromosome 9 is thus difficult to obtain routinely. However, as both Tm-2² gene and QTL9 are dominant or at least cumulative, the Tm-2² gene and QTL9 are advantageously found on the two distinct homologs of chromosome 9, i.e. they are both heterozygously present. In any event, the Tm-2² gene is preferably heterozygously present.

According an embodiment, the invention is thus directed to a plant, cell or seed comprising the QTL9 according to the invention, heterozygously, as well as the Tm-2² gene or an analog thereof providing ToMV and TMV resistances. The Tm-2² gene is well known to those skilled in the art; a suitable sequence for this gene is referred to as Solyc09g018220, or GeneBank AF536201, and AAQ10736 for the protein sequence. Variants and analogs are well known in the field of the invention. According to a preferred embodiment, the Tm-2² gene according to the invention is a gene encoding a protein having the 861 amino acid sequence reported in AAQ10736 (SEQ ID NO:114), or a protein having at least 75%, preferably at least 80%, sequence identity with this sequence and exhibiting the Tm-2² activity, namely the ability to inhibit viral RNA replication of a ToMV strain.

According to a preferred embodiment, a tomato plant, cell or seed of the invention thus comprises the QTL9 and the Tm-2² gene or a variant thereof, both heterozygously, and the QTL11 of the invention, homozygously or heterozygously, the combination providing ToBRFV, ToMV and TMV resistances. Further resistances may also be added if appropriate.

As disclosed in the application PCT/IB2019/00674 in the name of the same Applicant, the presence of the Tm-1 gene, may also improve the ToBRFV resistance. A plant, cell or seed according to the present invention thus advantageously also comprises the Tm-1 gene. The Tm-1 gene is as defined inter alia in the publication Ishibashi et al, 2007 (An inhibitor of viral RNA replication is encoded by a plant resistance gene. PNAS Aug. 21, 2007 104 (34) 13833-13838); preferably ‘Tm-1 gene’ refers to a genetic sequence encoding a protein having the Tm-1 activity reported in the article, namely the ability to inhibit the viral replication of a wild-type ToMV strain Tm-1 sensitive, for example the strain ToMV-L disclosed in this article. According to a preferred embodiment, the Tm-1 gene according to the invention is a gene encoding a protein having the 754 amino acid sequence reported in Ishibashi et al, corresponding to SEQ ID No:115 (NCBI BAF75724) or a protein having at least 75%, preferably at least 80%, sequence identity with this sequence and exhibiting the Tm-1 activity reported in Ishibashi et al, 2007, namely the ability to inhibit viral RNA replication of a wild-type Tm-1 sensitive ToMV strain.

The invention thus also encompasses tomato plant, cell or seed comprising the Tm-1 gene, either homozygously or heterozygously, in addition to the QTL9 of the invention, and potentially the Tm-2² gene and the QTL11, or the QTL3 on chromosome 11 as defined in WO2018/219941. The invention thus also encompasses tomato plant, cell or seed comprising the Tm-1 gene, either homozygously or heterozygously, in addition to the QTL11 of the invention, preferably homozygously.

According to still another embodiment, a plant of the invention is used as a scion or as a rootstock in a grafting process. Grafting is a process that has been used for many years in crops such as cucurbitacea, but only more recently for tomato. Grafting may be used to provide a certain level of resistance to telluric pathogens such as Phytophthora or to certain nematodes. Grating is therefore intended to prevent contact between the plant or variety to be cultivated and the infested soil. The variety of interest used as the graft or scion, optionally an F1 hybrid, is grafted onto the resistant plant used as the rootstock. The resistant rootstock remains healthy and provides, from the soils, the normal supply for the graft that it isolates from the diseases.

As detailed above, the invention is directed to S. lycopersicum plants, exhibiting the improved ToBRFV resistance, as well as to seeds giving rise to those plants, and cells of these plants or seeds, or other plant parts, comprising the resistance QTL9 and/or QTL11 in their genome, introgressed from S. pimpinellifolium and to progeny of such a plant of the invention comprising said QTL.

Progeny encompasses the first, the second, and all further descendants from a cross with a plant according to the invention, wherein a cross comprises a cross with itself or a cross with another plant. A plant or seed according to the invention may be a progeny or offspring of a plant grown from the deposited seeds LVSTBRFVRES2, deposited at the NCIMB under the accession number NCIMB 43591. Plants grown from the deposited seeds are indeed homozygous for the QTL9 of the invention conferring the improved phenotype, as well as for the QTL11; they thus bear in their genome the QTLs of interest on each of the homologues of chromosome 9 and 11. They can be used to transfer these sequences into another background by crossing and selfing and/or backcrossing.

The invention is also directed to the deposited seeds of LVSTBRFVRES2 (NCIMB 43591) and to plants grown from one of these seeds, containing homozygously the QTL9 conferring the phenotype of interest, as well as the QTL11. It is noted that these seeds do not correspond to a plant variety, they are not homozygous for most of the genes except the QTLs of the invention; their phenotype is thus not fixed during propagation, except for the ToBRFV resistance/tolerance of the invention; most of their phenotypic traits segregate during propagation, with the exception of ToBRFV resistance of the invention.

The invention is also directed to plants or seeds as defined above, i.e. containing the QTL9 and/or QTL11 of interest in homozygous or heterozygous state, said sequences conferring the improved phenotype, potentially in combination, which plants or seeds are obtainable by transferring the QTLs from a S. lycopersicum plant, representative seeds thereof were deposited under NCIMB accession NCIMB-43591, into another S. lycopersicum genetic background, for example by crossing said plant with a second tomato plant parent and selection of the plant bearing the QTL9 and/or QTL11 responsible for the phenotype of interest. In such crossing, the QTL9, as well as QTL11 if appropriate, can be transferred.

It is noted that the seeds or plants of the invention may be obtained by different processes, and are not exclusively obtained by means of an essentially biological process.

According to such an aspect, the invention relates to a tomato plant or seed, preferably a non-naturally occurring tomato plant or seed, which may comprise one or more mutations in its genome, which provides the plant with a fruit and/or a foliar resistance to Tomato Brown Rugose Fruit virus, which mutation is as present, for example, in the genome of plants of which a representative sample was deposited with the NCIMB under deposit number NCIMB 43591.

In another embodiment, the invention relates to a method for obtaining a tomato plant or seed carrying one or more mutations in its genome, which provides the plant with a fruit and/or a foliar resistance to Tomato Brown Rugose Fruit virus. Such a method is illustrated in example 7 and may comprise:
a) treating M0 seeds of a tomato plant to be modified with a mutagenic agent to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M1+n seeds.

The M1+n seeds are grown into plants and submitted to ToBRFV infection. The surviving plants, or those with the milder symptoms of ToBRFV infection, are multiplied one or more further generations while continuing to be selected for their fruit and/or foliar resistance to ToBRFV.

In this method, the M1 seeds of step a) can be obtained via chemical mutagenesis such as EMS mutagenesis. Other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea (enu), and sodium azide.

Alternatively, the mutations are induced by means of irradiation, which is for example selected from x-rays, fast neutrons, UV radiation.

In another embodiment of the invention, the mutations are induced by means of genetic engineering. Such mutations also include the integration of sequences conferring the ToBRFV fruit and/or foliar resistance, as well as the substitution of residing sequences by alternative sequences conferring the ToBRFV fruit and/or foliar resistance or tolerance. Preferably, the mutations are the integration of QTL9 and/or QTL11, as described above, in replacement of the homologous sequences of a S. lycopersicum plants. Even more preferably, the mutation is the substitution of the sequence comprised within SNP TO-0201220 (SEQ ID NO:1) and SNP TO-0201233 (SEQ ID NO:14) on chromosome 9 of S. lycopersicum genome, or a fragment thereof, by the homologous sequence on chromosome 9 present in the genome of a plant of which a representative sample was deposited with the NCIMB under deposit number NCIMB 43591, wherein the sequence or fragment thereof confers resistance to ToBRFV. According to another embodiment, the mutation is the substitution of the sequence comprised within SNP TO-0201237 (SEQ ID NO:102) and SL2.50ch11_9924232 (SEQ ID NO:115) on chromosome 11 of S. lycopersicum genome, or a fragment thereof such as the sequence comprised within SNP TO-0201237 and SNP TO-0201241, by the homologous sequence on chromosome 11 present in the genome of a plant of which a representative sample was deposited with the NCIMB under deposit number NCIMB 43591, wherein the sequence or fragment thereof confers resistance to ToBRFV when present homozygously.

The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated through the use of Zinc finger nuclease (ZFN) technology (ZFN-1, ZFN-2 and ZFN-3, see U.S. Pat. No. 9,145,565), Oligonucleotide directed mutagenesis (ODM), Cisgenesis and intragenesis, Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease re-engineered homing endonucleases, DNA guided genome editing (Gao et al., Nature Biotechnology (2016)), and Synthetic genomics. A major part of targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”.

The resistance, or intermediate resistance, against ToBRFV corresponds, according to the invention, to an important reduction of the non-marketable fruits of the resistant plants of the invention; Especially, resistant plants as disclosed herein bear tomatoes such that at least 70% of the fruits are marketable at maturity, i.e. have no discoloration spots, no browning calyx, are not undersized with a rough surface and have no brown, necrotic spots. Preferably, at least 80% of the fruits remain marketable at maturity, preferably at least 90% of the fruits, even in case of a double infection.

As defined above, a plant of the invention is characterized by the presence of introgressed sequences on chromosome 9, in a region of this chromosome delimited by SNP TO-0201220 and SNP having SEQ ID NO:101, and/or by the presence of introgressed sequences on chromosome 11, in a region of this chromosome delimited by SNP TO-0201237 and SL2.50ch11_9924232, preferably by SNP TO-0201237 and SNP TO-0201241. Introgressed sequences from S. pimpinellifolium may however be found beyond these boundaries or flanking sequences. Similarly, introgressed sequences are to be found within the region mentioned above, but the whole region is not necessarily made of introgressed sequences. In view of the markers identified and used by the inventors for QTL9, introgressed sequences, conferring the ToBRFV resistance are preferably to be found, in the genome of a plant, seed or cell of the invention, at least at one or more of the 101 loci encompassing the 101 SNPs having SEQ ID NO:1-101 mentioned in table H, and more preferably at least at one of the following 14 loci: the locus encompassing TO-0201220, the locus encompassing TO-0201221, the locus encompassing TO-0201222, the locus encompassing TO-0201223, the locus encompassing TO-0201224, the locus encompassing TO-0201225, the locus encompassing TO-0201226, the locus encompassing TO-0201227, the locus encompassing TO-0201228, the locus encompassing TO-0201229, the locus encompassing TO-0201230, the locus encompassing TO-0201231, the locus encompassing TO-0201232 and the locus encompassing TO-0201233 on chromosome 9.

By “a locus encompassing a SNP marker”, it is meant the sequences around the polymorphism of the SNP, preferably the sequence extending from about 2 megabases upstream to about 2 megabases downstream the SNP, preferably 1 megabase, preferably even 0.5 megabases upstream and downstream the SNP.

The introgressed sequences at these loci are those to be found at the corresponding loci in the seeds LVSTBRFVRES2 corresponding to NICMB43591.

For QTL11, introgressed sequences, conferring the ToBRFV resistance when present homozygously are preferably to be found, in the genome of a plant, seed or cell of the invention, at least at one or more of the 14 loci encompassing the 14 markers having SEQ ID NO:102-115 mentioned in table K. The introgressed sequences at these loci are also those to be found at the corresponding loci in the seeds LVSTBRFVRES2 corresponding to NICMB43591.

The invention in another aspect also concerns any plant likely to be obtained from seed or plants of the invention as described above, and also plant parts of such a plant, and most preferably explant, scion, cutting, seed, fruit, root, rootstock, pollen, ovule, embryo, protoplast, leaf, anther, stem, petiole, cotyledon, flower, root tip, hypocotyl and any other plants part, wherein said plant, explant, scion, cutting, seed, fruit, root, rootstock, pollen, ovule, embryo, protoplast, leaf, anther, stem, petiole, cotyledon, flower, root tip, hypocotyl and/or plant part is obtainable from a seed or plant according to the first aspect of the invention, i.e. bearing the QTL9 and/or QTL11 of interest, homozygously or heterozygously in their genome. These plant parts, inter alia explant, scion, cutting, seed, fruit, root, rootstock, pollen, ovule, embryo, protoplast, leaf, anther, stem, petiole, cotyledon, flower, root tip or hypocotyl, comprise in their genome the QTL9 and/or QTL11 conferring the phenotype of interest, i.e. resistance to ToBRFV, especially fruit resistance for QTL9 and foliar resistance for QTL11.

According to a preferred embodiment, the invention is directed to seed as described above, which develops into a plant according to the first aspect of the invention, thus resistant against ToBRFV infection thanks to the presence of the resistance QTL9, or QTL11, or introgressed sequences, as defined above.

The QTL9 and QTL11 referred to in this aspect of the invention are the ones defined above in the context of plants of the invention.

The plant part may advantageously comprise the QTL11 as defined above, in addition or in place of QTL9.

The different features of the QTLs defined in relation with the first aspect of the invention apply mutatis mutandis to this aspect of the invention. The QTL9 is thus preferably chosen from those present in the genome of a plant corresponding to the deposited material LVSTBRFVRES2 (NCIMB accession number 43591). It is advantageously characterized by the presence of at least one of the resistance alleles of the SNPs of table H; preferably by the presence of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and/or allele G of TO-020133, more preferably by allele G of SNP TO-0201220, allele G of SEQ ID TO-0201221, allele A of TO-0201229, allele C of TO-0201231 or allele G of TO-0201233, and even more preferably by allele G of SNP TO-0201220 and/or allele A of TO-0201229.

The QTL11 is preferably chosen from those present in the genome of a plant corresponding to the deposited material LVSTBRFVRES2 (NCIMB accession number 43591). It is advantageously characterized by the presence of at least one of the resistance alleles of the markers of table K; preferably by the presence of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232; e.g. by the presence of at least one of allele G of TO-0201237, allele A of TO-0201238, allele A of TO-0201239, allele A of TO-0201240 and allele A of TO-0201241.

The invention is also directed to cells of S. lycopersicum plants, such that these cells comprise, in their genome, the QTL9 or QTL11 of the present invention conferring independently the phenotype of interest to a S. lycopersicum plant, and potentially QTL9 and QTL11. The QTL9, as well as the QTL11, is the one already defined in the frame of the present invention, it is characterized by the same features and preferred embodiments already disclosed with respect to the plants and seeds according to the preceding aspects of the invention. The presence of this QTL, i.e. QTL9 or QTL11 can be revealed by the techniques disclosed above and well known to the skilled reader. It can inter alia be determined whether the QTL is present homozygously or heterozygously in the genome of such a cell of the invention. The QTL9 is advantageously characterized by the presence of at least one of the resistance alleles of the SNPs of table H; preferably by the presence of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and/or allele G of TO-020133, more preferably by allele G of SNP TO-0201220, allele G of SEQ ID TO-0201221, allele A of TO-0201229, allele C of TO-0201231 or allele G of TO-0201233, and even more preferably by allele G of SNP TO-0201220 and/or allele A of TO-0201229. The QTL11 is advantageously characterized by the presence of at least one of the resistance alleles of the markers of table K preferably by the presence of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232; e.g. by the presence of at least one of allele G of TO-0201237, allele A of TO-0201238, allele A of TO-0201239, allele A of TO-0201240 and allele A of TO-0201241.

Cells according to the invention can be any type of S. lycopersicum cell, inter alia an isolated cell and/or a cell capable of regenerating a whole S. lycopersicum plant, bearing the QTL9 and/or QTL11 of interest.

The present invention is also directed to a tissue culture of non-regenerable or regenerable cells of the plant as defined above according to the present invention; preferably, the regenerable cells are derived from embryos, protoplasts, meristematic cells, callus, pollen, leaves, anthers, stems, petioles, roots, root tips, fruits, seeds, flowers, cotyledons, and/or hypocotyls of the invention, and the cells contain the QTL9 and/or QTL11 in their genome conferring independently the improved phenotype, namely fruit resistance to ToBRFV for QTL9 and foliar resistance to ToBRFV for QTL11. Preferably, such a cell comprises the QTL9, and also comprises the QTL11, as defined in the context of the present invention, either homozygously or heterozygously.

Such a cell also advantageously comprises any additional resistance or tolerance gene, as disclosed in the context of the first aspect of the invention, also applicable here.

The tissue culture will preferably be capable of regenerating plants having the physiological and morphological characteristics of the foregoing tomato plant, and of regenerating plants having substantially the same genotype as the foregoing tomato plant. The present invention also provides tomato plants regenerated from the tissue cultures of the invention.

The invention also provides a protoplast of the plant defined above, or from the tissue culture defined above, said protoplast containing the QTL9 and/or the QTL11, conferring the improved phenotype of the invention.

The invention is also directed to tissue of a plant of the invention; the tissue can be an undifferentiated tissue, or a differentiated tissue. Such a tissue comprises one or more cells comprising the QTL of the invention.

The invention is also directed to propagation material, capable of producing a resistant tomato plant according to the invention, comprising the introgressed sequences or QTL as defined above.

According to another aspect, the present invention is also directed to the use of a tomato plant of the invention, preferably comprising homozygously the QTL9 of the invention, as a breeding partner in a breeding program for obtaining S. lycopersicum plants having the improved phenotype of the invention. Indeed, such a breeding partner harbors homozygously in its genome the QTL9 conferring the phenotype of interest. By crossing this plant with a tomato plant, especially a line, it is thus possible to transfer the QTL9 of the present invention conferring the desired phenotype, to the progeny. A plant according to the invention can thus be used as a breeding partner for introgressing the QTL9 conferring the desired phenotype into a S. lycopersicum plant or germplasm, namely ToBRFV resistance. Although a plant or seed bearing the QTL9 of interest heterozygously, can also be used as a breeding partner as detailed above, the segregation of the phenotype is likely to render the breeding program more complex.

The improved phenotype of the invention is resistance to ToBRFV, inter alia fruit resistance or foliar resistance, or fruit and foliar resistance.

The breeding partner may also comprise the QTL11 as defined in the invention, preferably homozygously.

The introgressed QTL9 will advantageously be introduced into varieties that contain other desirable genetic traits such as resistance to disease, early fruit maturation, drought tolerance, fruit shape, and the like. Preferably, the introgressed QTL9 will advantageously be introduced into plants or varieties comprising the Tm-2² gene.

According to another embodiment, the invention is also directed to the same uses of a tomato plant of the invention, but comprising homozygously the QTL11 of the invention, as a breeding partner.

The invention is also directed to the same use with plants or seed of LVSTBRFVRES2, deposited at the NCIMB under the accession number NCIMB 43591, and to plants derived therefrom, comprising the QTL9 and/or the QTL11 homozygously. Said plants are also suitable as introgression partners in a breeding program aiming at conferring the desired phenotype to a S. lycopersicum plant or germplasm.

In such a breeding program, the selection of the progeny displaying the desired phenotype, or bearing the QTL9 linked to the desired phenotype, can advantageously be carried out on the basis of the alleles of the SNP markers, especially the SNP markers of the invention having SEQ ID NO:1-101.

For QTL9, a progeny of the plant is preferably selected on the presence of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and/or allele G of TO-020133, more preferably of allele G of SNP TO-0201220, allele G of SEQ ID TO-0201221, allele A of TO-0201229, allele C of TO-0201231 or allele G of TO-0201233, and even more preferably of allele G of SNP TO-0201220 and/or allele A of TO-0201229.

The selection can alternatively be made on the basis of the presence of any one of the resistant alleles of the 101 SNPs of the invention linked to the improved phenotype or a combination of these alleles.

Such selection will be made on the presence of the alleles of interest in a genetic material sample of the plant to be selected. The presence of this or these allele(s) indeed confirms the presence of the QTL9, or introgressed sequences, of the invention, at the loci defined by said SNPs. Following point mutation or recombination event, it is however conceivable that at least 1 or 2 of these alleles is lost, the remaining of the chromosomal fragment bearing the QTL9 of interest still conferring the phenotype of interest.

The selection of the progeny bearing the QTL11 can advantageously be carried out on the basis of the alleles of the makers having SEQ ID NO:102-115, preferably the alleles of the SNP markers having SEQ ID NO:102-111, preferably on the basis of the presence of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232; e.g. by the presence of at least one of allele G of TO-0201237, allele A of TO-0201238, allele A of TO-0201239, allele A of TO-0201240 and allele A of TO-0201241.

A plant according to the invention, or grown from a seed as deposited under accession number NCIMB 43591, is thus particularly valuable in a marker assisted selection for obtaining commercial tomato lines and varieties, having the improved phenotype of the invention.

The invention is also directed to the use of said plants in a program aiming at identifying, sequencing and/or cloning the genetic sequences conferring the desired phenotype.

Any specific embodiment described for the previous aspects of the invention is also applicable to this aspect of the invention, especially with regard to the features of the QTL9 and QTL11 conferring the phenotype of interest.

According to still another aspect, the invention also concerns methods or processes for the production or breeding of S. lycopersicum plants, having the desired phenotype, especially commercial plants and inbred parental lines. The present invention is indeed also directed to transferring the QTL, or introgressed sequences of the invention conferring the ToBRFV resistance to other tomato plants, especially other tomato varieties, or other species or inbred parental lines, and is useful for producing new types and varieties of tomato.

In this regard, the invention also comprises methods for breeding S. lycopersicum plants having ToBRFV resistance, comprising the steps of crossing a plant grown from the deposited seeds LVSTBRFVRES2 NCIMB 43591 or progeny thereof bearing the QTL9 of the invention conferring ToBRFV resistance, with an initial S. lycopersicum plant preferably devoid of said QTL. The QTL is as defined above, namely introgressed from S. pimpinellifolium and is preferably present in the genome of the seeds of LVSTBRFVRES2, NCIMB accession number 43591. This QTL is identifiable by at least one of the resistant alleles of the SNP marker having SEQ ID NO: 1-101.

The invention also concerns a method for conferring ToBRFV resistance to a S. lycopersicum plant, comprising genetically modifying said plant to introduce the resistance QTL9 of the invention. Said QTL9 is as defined above and is preferably present in the genome of the seeds of LVSTBRFVRES2, NCIMB accession number 43591. The genetic modification can be carried out by any methods or means well known to the skilled person.

The invention also concerns the same methods for breeding S. lycopersicum plants having ToBRFV resistance and for conferring ToBRFV resistance to a S. lycopersicum plant, in connection with QTL11 instead of QTL9. In such a case, the QTL is identifiable by at least one of the resistant alleles of the markers having SEQ ID NO: 102-115.

This invention is thus directed to a method for breeding S. lycopersicum plants having resistance against ToBRFV, comprising the steps of crossing a plant grown from the deposited seeds NCIMB 43591 or progeny thereof bearing the QTL9 on chromosome 9 introgressed from S. pimpinellifolium and conferring ToBRFV resistance, with an initial S. lycopersicum plant devoid of said QTL9, wherein said QTL9 on chromosome 9 is present in the genome of the seeds of plant LVSTBRFVRES2, NCIMB accession number 43591, and is identifiable by allele G of SNP TO-0201220, allele G of SEQ ID TO-0201221, allele A of TO-0201229, allele C of TO-0201231 or allele G of TO-0201233. The invention is also directed to a method for breeding S. lycopersicum plants having resistance against ToBRFV, comprising the steps of crossing a plant grown from the deposited seeds NCIMB 43591 or progeny thereof bearing the QTL11 on chromosome 11 introgressed from S. pimpinellifolium and conferring ToBRFV resistance, with an initial S. lycopersicum plant devoid of said QTL11, wherein said QTL11 on chromosome 11 is present in the genome of the seeds of plant LVSTBRFVRES2, NCIMB accession number 43591, and is identifiable by allele G of TO-0201237, allele A of TO-0201238, allele A of TO-0201239, allele A of TO-0201240, allele A of TO-020124, allele CT of SL2.50ch11_9684449, allele AT of SL2.50ch11_9779896, allele C of SL2.50ch11_9823405 or allele GT of SL2.50ch11_9924232.

Specifically, the invention also concerns a method or process for the production of a plant having ToBRFV resistance comprising the following steps:
a) Crossing a plant grown from a deposited seed NCIMB 43591, or progeny thereof, comprising the QTL9 conferring ToBRFV resistance, and an initial S. lycopersicum plant, preferably devoid of said QTL,
b) Selecting one plant in the progeny thus obtained, comprising the QTL9 of the present invention;
c) Optionally self-pollinating one or several times the plant obtained at step b) and selecting in the progeny thus obtained a plant having resistance to ToBRFV, whether a fruit resistance, a foliar resistance or both.

Alternatively, the method or process may comprise instead of step a) the following steps:
a1) Crossing a plant corresponding to the deposited seeds (NCIMB 43591), or progeny thereof, comprising the QTL9 conferring ToBRFV resistance, and an initial S. lycopersicum plant, preferably devoid of said QTL,
a2) Increasing the F1 hybrid by means of selfing to create F2 population.

In the above methods or processes, SNPs markers are preferably used in steps b) and/or c), for selecting plants bearing sequences conferring the resistance phenotype of interest.

The SNP markers are preferably one or more of the 101 SNP markers of the invention having SEQ ID NO:1 to 101, including all combinations thereof as mentioned elsewhere in the present application and preferably SNPs having SEQ ID NO:1-14.

By selecting a plant on the basis of the allele of one or more SNPs, it is to be understood that the plant is selected as having ToBRFV resistance, whether a fruit tolerance/resistance, a foliar tolerance/resistance or both with respect to the initial plant, when the allele of the SNP(s) is (are) the allele corresponding to the allele of the LVSTBRFVRES2 parent for this SNP and not the allele of the initial S. lycopersicum plant. For example, a plant can be selected as having the improved phenotype of the invention, when allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and/or allele G of TO-020133 is detected, more preferably when allele G of SNP TO-0201220, allele G of SEQ ID TO-0201221, allele A of TO-0201229, allele C of TO-0201231, allele G of TO-0201232 or allele G of TO-0201233 is detected, and even more preferably when allele G of SNP TO-0201220 and/or allele A of TO-0201229 is detected. Any other resistant allele for the SNPs mentioned in table G can alternatively be used.

Preferably, the S. lycopersicum plant of step a) is an elite line, used in order to obtain a plant with commercially desired traits or desired horticultural traits. Advantageously such a plant is resistant to TMV, due to the presence of the Tm-2² gene homozygously or heterozygously.

A method or process as defined above may advantageously comprises backcrossing steps, preferably after step c), in order to obtain plants having all the characterizing features of S. lycopersicum plants. Consequently, a method or process for the production of a plant having these features may also comprise the following additional steps:
d) Backcrossing the resistant plant selected in step b) or c) with a S. lycopersicum plant;
e) Selecting a plant bearing the QTL9, or introgressed sequences, of the present invention.

The plant used in step a), namely the plant corresponding to the deposited seeds can be a plant grown from the deposited seeds; it may alternatively be any plant according to the 1^{st} aspect of the invention, bearing the QTL9 or introgressed sequences, conferring the phenotype, preferably bearing these sequences homozygously.

Preferably such a plant also comprises the QTL11 as defined, preferably homozygously.

At step e), SNPs markers can be used for selecting plants having ToBRFV resistance, with respect to the initial plant. The SNP markers are those of the invention, as described in the previous sections. According to a preferred embodiment, the method or process of the invention is carried out such that, for at least one of the selection steps, namely b), c) and/or e), the selection is based on the detection of at least one of the resistant alleles of SNPs having SEQ ID NO:1-101. The preferred alleles and combinations have already been disclosed and are applicable to this embodiment of the invention.

It is to be noted that, when plants having the improved phenotype, and bearing homozygously the QTL conferring this phenotype, are to be selected, the selection is to be made on the basis of one or more the SNPs of the invention, on the presence of the alleles representative of the QTL, namely the alleles LVSTBRFVRES2 parent, in combination with the absence of the alleles representative of the recurrent susceptible S. lycopersicum parent.

The selection can also be made on the basis of any other marker linked to the introgressed sequences and representative of the presence of these introgressed sequences by opposition to the resident sequences of the susceptible parent. Methods for defining alternative markers are also in the scope of the present invention and disclosed in another section.

The plant selected at step e) is preferably a commercial plant, especially a plant having fruits which weight at least 10 g but preferably 25 g, at least 100 g, at least 150 g or at least 200 g at full maturity in normal culture conditions.

Preferably, steps d) and e) are repeated at least twice and preferably three times, not necessarily with the same S. lycopersicum plant. Said S. lycopersicum plant is preferably a breeding line. Resistance to nematode trait or resistance to ToMV may additionally be selected, at each selection step of the processes disclosed above.

The self-pollination and backcrossing steps may be carried out in any order and can be intercalated, for example a backcross can be carried out before and after one or several self-pollinations, and self-pollinations can be envisaged before and after one or several backcrosses.

The selection of the progeny having the desired improved phenotype can also be made on the basis of the comparison of the ToBRFV resistance from the S. lycopersicum parent, through protocols as disclosed inter alia in the examples; the tested resistance/tolerance can be either fruit resistance/tolerance or foliar resistance/tolerance, or both.

The method used for allele detection can be based on any technique allowing the distinction between two different alleles of a SNP, on a specific chromosome.

The invention is also directed to the same methods, wherein at step a), a plant grown from a deposited seed NCIMB 43591, or progeny thereof, comprising the QTL11 conferring ToBRFV resistance is used. All detection/selection steps are then carried out with respect to QTL11, especially with the markers having SEQ ID NO:102-115, and more preferably on the basis of the presence of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and/or allele GT of SL2.50ch11_9924232; e.g. by the presence of at least one of allele G of TO-0201237, allele A of TO-0201238, allele A of TO-0201239, allele A of TO-0201240 and allele A of TO-0201241. Alternative preferred lists of markers and of resistant alleles have already been disclosed above.

The invention thus is also directed to a method for conferring resistance to ToBRFV to S. lycopersicum plants, comprising the steps of:
a) Crossing a plant grown from the deposited seeds NCIMB 43591, or progeny thereof, bearing the QTL9 on chromosome 9 and/or the QTL11 on chromosome 11, introgressed from S. pimpinellifolium and conferring independently ToBRFV resistance in NCIMB 43591, and an initial S. lycopersicum plant preferably devoid of said QTL(s),
b) Selecting a plant in the progeny thus obtained, bearing the QTL9 and/or the QTL11;
c) Optionally self-pollinating one or several times the plant obtained at step b) and selecting in the progeny thus obtained a plant having resistance to ToBRFV.

According to another embodiment, the invention is directed to a method for conferring resistance to ToBRFV to S. lycopersicum plants, comprising the steps of:
a1) Crossing a plant grown from the deposited seeds NCIMB 43591 or progeny thereof, bearing the QTL9 on chromosome 9 and/or the QTL11 on chromosome 11, introgressed from S. pimpinellifolium and independently conferring ToBRFV resistance in NCIMB 43591, and an initial S. lycopersicum plant, preferably devoid of said QTL(s), thus generating the F1 population,
a2) Selfing the F1 hybrids to create F2 population,
b) Selecting individuals in the progeny thus obtained having resistance to ToBRFV.

SNPs markers are advantageously used in steps b) and/or c) for selecting plants bearing the QTL9 and/or the QTL11 conferring independently ToBRFV resistance.

The invention is also directed to a method for obtaining commercial tomato plants or inbred parental lines thereof, having the desired improved phenotype, corresponding to a fruit and/or foliar tolerance and/or resistance to the Tomato Brown Rugose Fruit virus, with respect to an initial commercial S. lycopersicum plant, comprising the steps of:
a) Backcrossing a plant obtained by germinating a deposited seed LVSTBRFVRES2 NCIMB accession number 43591, or progeny thereof, bearing the QTL9 conferring ToBRFV resistance, with a commercial S. lycopersicum plant,
b) Selecting a plant bearing the QTL9 of the present invention.

Preferably, the selection is made on the basis of one or more of the 101 SNPs of the invention, as detailed for the other methods of the invention.

Alternatively, the progeny of step a) is a progeny bearing the QTL11, and the selection of step b) is based on QTL11, preferably on the basis of one or more of the 14 markers of the invention having SEQ ID NO:102-115, as detailed for the other methods of the invention.

In all the methods and processes of the invention according to the invention, the initial S. lycopersicum plant is determinate, indeterminate or semi-determinate.

As already disclosed, the tomato plants according to the invention are preferably also resistant to Tomato Mosaic Virus, to nematodes, to TYLCV and to Fusarium and Verticillium. In order to obtain such plants in the processes and methods of the invention, the S. lycopersicum parents used in the breeding schemes are preferably bearing sequences conferring resistance to Tomato Mosaic Virus, to nematodes, to TYLCV and to Fusarium and Verticillium; and the selection steps are carried out to select plants having these resistance sequences, in addition to the QTL(s) conferring the improved phenotype of the invention.

The present invention is also directed to a S. lycopersicum plant and seed obtained or obtainable by any of the methods and processes disclosed above. Such a plant is indeed a S. lycopersicum plant having the improved phenotype according to the first aspect of the invention. The seed of such S. lycopersicum are preferably coated or pelleted with individual or combined active species such as plant nutrients, enhancing microorganisms, or products for disinfecting the environment of the seeds and plants. Such species and chemicals may be a product that promotes the growth of plants, for example hormones, or that increases their resistance to environmental stresses, for example defense stimulators, or that stabilizes the pH of the substrate and its immediate surroundings, or alternatively a nutrient.

They may also be a product for protecting against agents that are unfavorable toward the growth of young plants, including herein viruses and pathogenic microorganisms, for example a fungicidal, bactericidal, hematicidal, insecticidal or herbicidal product, which acts by contact, ingestion or gaseous diffusion; it is, for example, any suitable essential oil, for example extract of thyme. All these products reinforce the resistance reactions of the plant, and/or disinfect or regulate the environment of said plant. They may also be a live biological material, for example a nonpathogenic microorganism, for example at least one fungus, or a bacterium, or a virus, if necessary with a medium ensuring its viability; and this microorganism, for example of the Pseudomonas, Bacillus, Trichoderma, Clonostachys, Fusarium, Rhizoctonia, etc. type stimulates the growth of the plant, or protects it against pathogens.

In all the previous methods and processes, the identification of the plants bearing the QTL, or introgressed sequences, responsible for the ToBRFV resistance, could be done by the detection of at least one of the alleles of the SNPs associated with the resistance QTL9, potentially in combination with the absence of the other allelic form of the SNPs of the present invention, in order to confirm the homozygous state of the QTL if needed. As such, the identification of a plant bearing homozygously QTL or introgressed sequences of the present invention will be based on the identification of at least one of the resistant alleles of SNPs having SEQ ID NO:1-101 for QTL9, as well as the absence of the susceptible allele of said SNP. For example, the identification of a plant bearing homozygously the QTL9 of the present invention will be based on the identification of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and/or allele G of TO-020133, as well as the absence of the corresponding susceptible allele, namely allele A of SNP TO-0201220, allele A of TO-0201221, allele G of TO-0201222, allele G of TO-0201223, allele C of TO-0201224, allele G of TO-0201225, allele G of TO-0201226, allele A of TO-0201227, allele G of TO-0201228, allele G of TO-0201229, allele A of TO-0201230, allele A of TO-0201231, allele A of TO-0201232 and/or allele A of TO-0201233.

Similarly, the identification of a plant bearing homozygously QTL or introgressed sequences of the present invention will be based on the identification of at least one of the resistant alleles of the markers having SEQ ID NO:102-115 for QTL11, as well as the absence of the susceptible allele of said SNP. For example, the identification of a plant bearing homozygously the QTL11 of the present invention will be based on the identification of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and/or allele GT of SL2.50ch11_9924232 as well as the absence of the corresponding susceptible allele, namely allele A of TO-0201237, allele T of TO-0201238, allele C of TO-0201239, allele C of TO-0201240, allele C of TO-0201241, allele C of SL2.50ch11_9684449, allele A of SL2.50ch11_9779896, allele T of SL2.50ch11_9823405 and allele G of SL2.50ch11_9924232.

The invention is also directed to the use of the information provided herewith by the present inventors, namely the existence of a QTL9 and of a QTL11, present in the deposited seeds of LVSTBRFVRES2, and conferring the improved phenotype to S. lycopersicum plants, and the disclosure of molecular markers associated to these QTLs or introgressed sequences. This knowledge can be used inter alia for precisely mapping the QTLs, for defining their sequence, for identifying tomato plants comprising the QTL conferring the improved phenotype and for identifying further or alternative markers associated to these QTLs. Such further markers are characterized by their location, namely close to the 101 markers disclosed in the present invention, and preferably from the 14 SNPs having SEQ ID NO:1-14 for QTL9, and by their association with the ToBRFV resistance revealed by the invention. For QTL11, the applicable markers are those having SEQ ID NO:102 to 115.

In this regard, the invention also concerns a method for identifying, detecting and/or selecting S. lycopersicum plants having the QTL9 of the present invention as found in the genome of the seeds of LVSTBRFVRES2 (NCIMB accession number 43591), said QTL conferring an improved resistance to ToBRFV with respect to a corresponding plant devoid of said sequences, the method comprising the detection of at least one of the resistant alleles of the SNP markers of table H, inter alia one of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-020133, more preferably one of allele G of SNP TO-0201220, allele G of SEQ ID TO-0201221, allele A of TO-0201229, allele C of TO-0201231 and allele G of TO-0201233, for example one of allele G of SNP TO-0201220 and allele A of TO-0201229 in a genetic material sample of the plant to be identified and/or selected. Preferably, at least 2 or 3, or 5 of the resistant alleles of SNPs having SEQ ID NO: 1-101 are to be detected.

The invention is also directed to a method for detecting or selecting S. lycopersicum plants having the QTL9 conferring resistance to ToBRFV and having at least one of the resistant alleles of the SNPs having SEQ ID NO:1-101, especially those having SEQ ID NO:1-14, wherein the detection or selection is made on condition of ToBRFV infection comprising inoculation of ToBRFV on the plants to be tested, either natural infection of artificial infection. The presence of the phenotype of interest is informative of the presence of the QTL9 or introgressed sequences of the invention, especially in a breeding scheme comprising a parent bearing the QTL9 of the invention.

The invention also concerns the same methods for identifying, detecting and/or selecting S. lycopersicum plants having the QTL11 of the present invention as found in the genome of the seeds of LVSTBRFVRES2, said QTL conferring an improved resistance to ToBRFV with respect to a corresponding plant devoid of said sequences, the method comprising the detection of at least one of the resistant alleles of the markers of table K, inter alia one of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232, in a genetic material sample of the plant to be identified and/or selected. Preferably, at least 2 or 3, or 5 of the resistant alleles of SNPs having SEQ ID NO: 102-115 are to be detected, or 2, 3 or 4 of the 9 markers TO-0201237-TO-0201241 and SL2.50ch11_9684449, SL2.50ch11_9779896, SL2.50ch11_9823405 and SL2.50ch11_9924232. According to another embodiment, at least one, 2 or 3 of the resistant alleles of the markers SL2.50ch11_9684449, SL2.50ch11_9779896, SL2.50ch11_9823405 and SL2.50ch11_9924232 are to be detected. Alternative preferred lists of markers and of resistant alleles have already been disclosed above. The detection or selection can made on condition of ToBRFV infection. The presence of the phenotype of interest is informative of the presence of the QTL11 or introgressed sequences of the invention homozygously.

The invention is also directed to a method for detecting and/or selecting a S. lycopersicum plant, especially commercial tomato plants, having the QTL of the invention, comprising the detection of at least one of the resistant alleles mentioned above, namely
allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-0201233, preferably at least one of allele G of SNP TO-0201220, allele G of SEQ ID TO-0201221, allele A of TO-0201229, allele C of TO-0201231, allele G of TO-0201232 or allele G of TO-0201233, for the QTL on chromosome 9, or
allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232 for the QTL on chromosome 11, for example at least one of allele G of TO-0201237, allele A of TO-0201238, allele A of TO-0201239, allele A of TO-0201240 or allele A of TO-0201241, in a genetic material sample of the plant to be selected.

Alternative preferred lists of resistant alleles have already been disclosed above.

The method is particularly adapted in a breeding program with LVSTBRFVRES2 (NCIMB accession number 43591), as initial parent, or progeny thereof, comprising the QTL of the invention conferring ToBRFV resistance.

The invention is further directed to a method for detecting and or selecting S. lycopersicum plants having the QTL9 and/or QTL11 of the present invention conferring ToBRFV resistance, on the basis of the detection of any molecular marker revealing the presence of said QTLs. Indeed, now that the QTL9 and QTL11 of the invention have been identified by the present inventors, the identification and then the use of molecular markers, in addition to the 101 SNPs of the invention (SEQ ID NO:1-101) or 14 markers (SEQ ID NO:102-115) can be easily achieved by a skilled artisan. The QTL9 may be characterized by the presence of at least one of the 101 SNPs of the invention, but it may also be identified through the use of different, alternative markers; the same applies to QTL11. Also included in the present invention are thus methods and uses of any such molecular markers for identifying the QTL of the invention in a tomato genome, wherein said QTL confers resistance to ToBRFV with respect to a corresponding plant devoid of said QTL, the QTL being characterized by the presence of the resistant allele of at least one of the SNPs having SEQ ID NO:1-101, preferably 1-14.

Are also included methods and uses of any such alternative molecular markers for identifying the QTL9 of the invention in a tomato genome, wherein said QTL confers ToBRFV resistance wherein said QTL is characterized by the presence of at least one of the resistant alleles of SNP having SEQ ID NO:1-101, preferably by one of allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-020133, more preferably one of allele G of SNP TO-0201220, allele G of SEQ ID TO-0201221, allele A of TO-0201229, allele C of TO-0201231 and allele G of TO-0201233, and even more preferably one of allele G of SNP TO-0201220 and allele A of TO-0201229.

Are also included methods and uses of any such alternative molecular markers for identifying the QTL11 of the invention in a tomato genome, wherein said QTL confers ToBRFV resistance when present homozygously wherein said QTL is characterized by the presence of at least one of the resistant alleles of markers having SEQ ID NO:102-115, preferably at least one of the resistant alleles of SNP having SEQ ID NO:102-111, more preferably by one of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232; for example one of allele G of TO-0201237, allele A of TO-0201238, allele A of TO-0201239, allele A of TO-0201240 and allele A of TO-0201241. Alternative preferred lists of resistant alleles have already been disclosed above.

The invention also concerns a method for detecting and/or selecting tomato plants having the resistance QTL as defined previously, conferring ToBRFV resistance, said method comprising:
a) Assaying tomato plants for the presence of at least one genetic marker genetically linked or associated to the QTL9 or QTL11 involved in ToBRFV resistance, especially conferring said resistance in tomato plants,
b) Selecting a plant comprising the genetic marker and the linked or associated QTL9 or QTL11 involved in ToBRFV resistance,
   wherein the QTL and the genetic marker are to be found in the genomic region delimited by TO-0201220 and the SNP having SEQ ID NO:101 for QTL9, preferably in the region delimited by TO-0201220 and TO-0201233 in the genome of S. lycopersicum, and in the genomic region delimited by the markers having SEQ ID NO:102 and 115 for QTL11, preferably in the region delimited by the markers having SEQ ID NO:102 and 111.

By association, or genetic association, and more specifically genetic linkage, it is to be understood that a polymorphism of a genetic marker (e.g. a specific allele of the SNP marker) and the phenotype of interest occur simultaneously, i.e. are inherited together, more often than would be expected by chance occurrence, i.e. there is a non-random association of the allele and of the genetic sequences responsible for the phenotype, as a result of their genomic proximity.

A genetic marker is either one of 101 markers disclosed above for QTL9 or an alternative marker, and is inherited with the phenotype of interest in preferably more than 90% of the meioses, preferably in more than 95%, 96%, 98% or 99% of the meioses. The same applies for QTL11.

The definition and preferred features of the QTL, or introgressed sequences, of the invention are as defined in other sections of the present specification. The QTL conferring the ToBRFV resistance is advantageously as found in the genome of the seeds LVSTBRFVRES2.

The invention thus concerns the use of one or more molecular or genetic markers, for fine-mapping or identifying a QTL in the tomato genome, conferring the ToBRFV resistance of the invention, wherein said one or more markers is/are localized in one of the following chromosomal regions:
in the chromosomal region delimited on chromosome 9 by SNP TO-0201220 (SEQ ID NO:1) and the SNP having SEQ ID NO:101,
at less than 2 megabase units from the locus of one of the 101 SNP markers of the invention, preferably from the locus of one of the SNPs having SEQ ID NO:1-14, and even more preferably from the locus of one of TO-0201220, TO-0201221, TO-0201229, TO-0201231 or TO-0201233.

According to a preferred embodiment, said one or more markers, as disclosed above, are in the chromosomal region delimited by TO-0201210 and the SNP having SEQ ID NO:101, or by TO-0201210 and TO-0201233, or by TO-0201221 and TO-0201233.

Said one or more molecular or genetic marker(s) is/are moreover preferably associated, with a p-value of 0.05 or less, with at least one of the following resistant alleles of the SNP having QED ID NO:1-101, for example with allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0202132 and/or allele G of TO-020133.

The molecular or genetic marker is preferably a SNP marker. It is more preferably at less than 1 megabase from the locus of at least one of the 101 SNPs of the invention, preferably at less than 0.5 megabase.

The p-value is preferably less than 0.01.

The invention moreover relates to the use of at least one of the 101 SNP markers of the invention, associated with the QTL on chromosome 9 conferring ToBRFV resistance, for identifying one or more alternative molecular or genetic markers associated with said QTL, wherein said one or more alternative molecular or genetic markers are:
in the chromosomal region delimited on chromosome 9 by SNP TO-0201220 (SEQ ID NO:1) and the SNP having SEQ ID NO:101,
at less than 2 megabase units from the locus of one of the 101 SNP markers of the invention, preferably from the locus of one of the SNPs having SEQ ID NO:1-14, and even more preferably from the locus of one of TO-0201220, TO-0201221, TO-0201229, TO-0201231 or TO-0201233.

According to a preferred embodiment, said alternative markers are in the preferred chromosomal regions mentioned above. The genetic association or linkage can advantageously be detected by following the alternative maker and the presence of the QTL in the progeny arising from a plant comprising the QTL of interest.

The alternative molecular markers are preferably associated with said QTL with a p-value of 0.05 or less, preferably less than 0.01. The QTL is preferably as be found in the genome of the deposited seeds NCIMB 43591.

A molecular or genetic marker and the resistance phenotype are inherited together in preferably more than 90% of the meioses, preferably more than 95%.

The molecular or genetic markers according to this aspect of the invention are preferably SNP. They are more preferably at less than 1 megabase from the locus of at least one of the 101 SNPs of the invention, preferably at less than 0.5 megabases.

The invention is also directed to the same methods and uses, wherein the marker(s) is/are localized:
in the chromosomal region delimited on chromosome 11 by the marker having SEQ ID NO:102 and the marker having SEQ ID NO:115,
at less than 2 megabase units from the locus of one of the 14 markers of table K, preferably from the locus of one of TO-0201237, TO-0201238, TO-0201239, TO-0201240, TO-0201241, SL2.50ch11_9684449, SL2.50ch11_9779896, SL2.50ch11_9823405 or SL2.50ch11_9924232.

Similarly, the invention also encompasses a method for identifying a molecular or genetic marker associated with a QTL conferring ToBRFV resistance to tomato plants, as described in the present application, comprising the steps of:
identifying a molecular or genetic marker in the genomic interval delimited by TO-0201220 and TO-0201233 or at less than 2 megabase units from the locus of one of the 101 SNPs of the invention, preferably less than 0.5 megabase units; and
determining whether an allele or state of said molecular or genetic marker is associated or linked with the phenotype of ToBRFV resistance in a segregating population issued from a plant exhibiting ToBRFV resistance, for example in a segregating population issued from a plant corresponding to the deposited seeds.

According to still another aspect, the invention is also directed to a method for genotyping a plant, preferably a S. lycopersicum plant or tomato germplasm, for the presence of at least one genetic marker associated with resistance or tolerance to ToBRFV infection, wherein the method comprises the determination or detection in the genome of the tested plant of a nucleic acid comprising at least one of the 101 markers of the invention, or comprising at least one of the alternative molecular markers as disclosed above. Preferably, the method comprises the step of identifying in a sample of the plant to be tested specific sequences associated with resistance to ToBRFV, in nucleic acid comprising at least one of resistant alleles of the SNPs of the invention.

According to a most preferred embodiment of this method, the method comprises the detection in the tested plant of the presence of nucleic acid comprising allele G of SNP TO-0201220 or allele A of SNP TO-0201229.

The invention also relates to the same methods in connection with QTL11 and with respect to the chromosomal region delimited on chromosome 11 by the marker having SEQ ID NO:102 and the marker having SEQ ID NO:115. The relevant markers or SNPs of this region have already been disclosed in the present invention, as well as preferred lists.

In view of the ability of the resistant plants of the invention to restrict the damages caused by ToBRFV infection, they are advantageously grown in an environment infested or likely to be infested or infected by ToBRFV; in these conditions, the resistant or tolerant plants of the invention produce more marketable tomatoes than susceptible plants. The invention is thus also directed to a method for improving the yield of tomato plants in an environment infested by ToBRFV comprising growing tomato plants comprising in their genome the QTL9 on chromosome 9 and/or QTL11 on chromosome 11, as defined according to the previous aspects of the invention, and conferring to said plants resistance to ToBRFV.

Preferably, the method comprises a first step of choosing or selecting a tomato plant comprising said QTL, or introgressed sequences, of interest. The method can also be defined as a method of increasing the productivity of a tomato field, tunnel or glasshouse, or as a method of reducing the intensity or number of chemical or fungicide applications in the production of tomatoes.

The invention is also directed to a method for reducing the loss on tomato production in condition of ToBRFV infestation or infection, comprising growing a tomato plant as defined above.

These methods are particularly valuable for a population of tomato plants, either in a field, in tunnels or in glasshouses.

Alternatively, said methods for improving the yield or reducing the loss on tomato production may comprise a first step of identifying tomato plants resistant/tolerant to ToBRFV and comprising in their genome the QTL9 and/or the QTL11 of the invention, that confers to said plants ToBRFV resistance, and then growing said resistant plants in an environment infested or likely to be infested by the virus. According to a preferred embodiment, the plants to be identified at the first step comprise allele A of TO-0201229, or at least one of the resistant alleles of the SNPs having SEQ ID NO:1-101.

The resistant plants of the invention are also able to restrict the growth of ToBRFV, thus limiting the infection of further plants and the propagation of the virus. Accordingly, the invention is also directed to a method of protecting a field, tunnel or glasshouse, or any other type of plantation, from ToBRFV infection, or of at least limiting the level of infection by ToBRFV of said field, tunnel or glasshouse or of limiting the spread of ToBRFV in a field, tunnel or glasshouse, especially in a tomato field. Such a method preferably comprises the step of growing a resistant or tolerant plant of the invention, i.e. a plant comprising in its genome the QTL9 on chromosome 9, conferring to said plant ToBRFV resistance. The plant of the invention to be used preferably comprises allele A of TO-0201229, or at least one of the resistant alleles of the SNPs having SEQ ID NO:1-101. According to another embodiment, the plant to be used comprises at least one of the resistant alleles of the markers having SEQ ID NO:102-115.

The invention also concerns the use of a plant resistant to ToBRFV for controlling ToBRFV infection or infestation in a field, tunnel or glasshouse, or other plantation; such a plant is a plant of the invention, comprising in its genome the QTL9 and/or QTL11, or introgressed sequences from S. pimpinellifolium on chromosome 9 or 11 as defined above. This use or method is also a method for disinfecting a field, tunnel or glasshouse by decreasing its viral population.

All the preferred features of the QTL are as defined in connection with the other aspects of the invention, namely it is preferably present in the seeds of LVSTBRFVRES2 (NCIMB accession number 43591), and it is identifiable by the SNP markers having SEQ ID NO:1-101 for QTL9; preferably SNP markers having SEQ ID NO:1-14, preferably by allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0202132 and/or allele G of TO-020133 for QTL9, and is identifiable by the markers having SEQ ID NO:102-115 for QTL11, preferably allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and/or allele GT of SL2.50ch11_9924232 for QTL11.

In still a further aspect, the invention also relates to a method of producing tomatoes comprising:
a) growing a S. lycopersicum plant of the invention, comprising the QTL9 and/or QTL11 as defined previously;
b) allowing said plant to set fruit; and
c) harvesting fruit of said plant, preferably at maturity and/or before maturity.

All the preferred embodiments regarding the QTL9 and QTL11 are already disclosed in the context of the previous aspects of the invention. The method may advantageously comprise a further step of processing said tomatoes into a tomato processed food.

### LEGEND OF THE FIGURES

FIG. 1: p-value plot of QTL associated to ToBRFV resistance, for the trait corresponding to AUDPC, based on F2 population (based on Source D and HMC1).

Vertical axis (y-axis) shows the −log 10 (p-value) and horizontal axis (x-axis) represents all SNPs by their positions (in physical distances bp) by chromosomes along the physical map.

FIG. 2A: adjusted values of fruit resistance: Turkey's test illustration

FIG. 2B: adjusted values of fruit resistance with confidence Intervals, depending on the presence of the alleles of the resistant parent, at QTL9

FIG. 3: Adjusted value for fruit resistance, of different genotypes

FIG. 4: fruit score repartition by QTL9 genotype

FIG. 5: adjusted values of leaves resistance with confidence Intervals

FIG. 6: Adjusted value for foliar resistance, of different genotypes with confidence intervals.

### EXAMPLES

### Example 1: Material and Methods

### 1.A. Validation Test for the Sources.

The test was carried out with 3 repetitions, of approximately 15 plants per tested accessions or genetic backgrounds. The plants were sown and infected at the 2-leaves stage. The scoring was then done, by visual assessment of the leaves, at 7, 14 and 28 days post infection.

For this test, the scale was as follows:
9: no symptoms
5: Moderate mosaic or/and necrosis symptoms
1: strong mosaic or/and necrosis symptoms

Elisa testing was done plants by plants for the 4 accessions, and in bulk for the two susceptible controls, at 32 days dpi.

### 1.B. Phenotyping for the F2 Population

The plants were sown and infected at the 2-leaves stage. The scoring was then done, by visual assessment of the leaves, at 7, 14 and 28 days post infection.

For this test, the scale was as follows:
9: no symptoms
7: light mosaic symptoms
5: medium mosaic symptoms
3: strong mosaic symptoms and/or bubbling
1: very strong mosaic symptoms and/or bubbling and/or deformation

For QTL analysis, different phenotypic variables/traits were used by the inventors, especially the note at 14 dpi, Note at 21 dpi, Note at 28 dpi, AUDPC.

The AUDPC (Area under the disease progress curve) was calculated by using the following formula:

$$AUDPC = {\sum }_{i = 1}^{n - 1} ( \frac{{y}_{i} + {y}_{i + 1}}{2} ) ( {t}_{i + 1} - {t}_{i} )$$

where “n” is the number of symptom assessments, “y” the symptom intensity (1 to 9) and “t” the time in dpi (days post inoculation).

### 1.3. DNA Extraction:

DNA was extracted from leaves ground using NucleoMag® Plant kit (Macherey-Nagel) according to the manufacturer's procedures. DNA purification was based on Magnetic-bead 10 technology for the isolation of genomic DNA from plant tissue. DNA concentrations were quantified with Quant-iT™ PicoGreen® dsDNA Assay Kit.

### 1.4. Protocol for Evaluation of ToBRFV Resistance Under Field Conditions

Inoculation stage: 10 and 17 days after planting. Plants were thus infected twice. At each time, inoculation of the 2 youngest leaves. In final, 4 different leaves are inoculated.

### Inoculum Preparation:

Isolate: ToBRFV Jordan local strain 2017. ToBRFV infected young leaves are taken from naturally infected plants, from plants resistant to TYLCV (Ty) and TMV, to avoid the presence of several viruses in the inoculum.

1 g of young leaves with ToBRFV symptoms is necessary to prepare 4 mL of inoculum, by grinding.

### Inoculum Verification and Inoculation:

Before inoculation, the presence of ToBRFV in the inoculum is checked by using a TMV immunostrip (this immunostrip is not specific and recognize also ToBRFV) and the absence of PepMV (Pepino) is check by using a PepMV immunotrip. Insofar as the infected plants are TMV resistant, the TMV immunostrip thus allows to detect ToBRFV presence in the inoculum.

The inoculum to be applied is positive with the TMV immunostrip and negative with the PepMV immunostrip.

The inoculum is applied on two young leaves of the plants to be tested, by rubbing gently this leaves with a rough sponge soaked in the inoculum.

### Symptoms Evaluation

The 1^{st} evaluation is carried out when the 1^{st} cluster of fruits is red and the second one is turning red.

The 2^{nd} evaluation is made when at least the 3^{rd} cluster is red.

Scale for the Leaves Symptoms:

9: No symptoms/7: weak symptoms on few leaves/5: medium symptoms on some leaves/3: medium symptoms on all leaves/1: strong symptoms on all leaves.

Scale for the Fruits Symptoms:
9: All fruits without any symptoms
7: light symptoms (discoloration) on one or few fruits
5: light/medium fruits discoloration on at least 2-3 fruits
3: medium/strong fruits discoloration and/or small fruits deformation on more than 30% of fruits.
1: very strong discoloration and/or medium/strong fruit deformation and/or fruits necrotic spots on more than 50% of fruits.

### 1.5. Protocol for Evaluation of Stemphylium Spp Resistance

Stemphylium spp is a plant pathogen fungus; it is the causal pathogen for gray leaf spot in tomatoes. The Sm gene, from Lycopersicum pimpinellifolium provides a genetic dominant resistance to Stemphylium.

### Inoculum Preparation:

Stemphylium, Sicilian strain, is stored at −80° C. The inoculum is prepared directly from the cryopreserved tubes, after culture on Petri dishes, with V8 medium. The conidia are obtained by scratching the surface of the medium and suspended in water with 1% glucose and then filtered on muslin. A solution comprising between 104 and 105 conidia per mL is obtained.

### Inoculation:

The plantlets to be tested are at the stage of 3 unfolded leaves, corresponding to 17 to 24 days after seeding. The inoculum is applied on by spraying on all the leaf surface, until formation of drops.

### Symptom Evaluation:

The scoring was then done, by visual assessment of the leaves, at 7 to 8 days post infection. For this test, the scale was as follows:
9: no symptoms
7: some brown necrotic lesions, less numerous than on susceptible plants
1: brown necrotic lesions, small or larger, on both faces of the leaves.

### Example 2: Identification of a Donor for ToBRFV Resistance and Preliminary Mapping of the QTLs

### Identification of a Suitable Wild Donor for the Resistance to ToBRFV

More than 500 different wild accessions were screened by the inventors in order to identify a potential source of resistance to ToBRFV, with a view to potentially introgressing the sequences conferring the resistance in these wild accessions, into S. lycopersicum background, especially in commercial plants.

Although the type of resistance which was expected by the inventors was fruit tolerance/resistance, such a test cannot be applied with wild accessions, having different fruit sizes and forms. The inventors therefore decided to rank the sources on a leaf symptoms test, as a surrogate for fruit resistance as this parameter cannot be screened.

Among the wild accessions screened, only 4 potential sources were identified (0.9%), in the S. habrochaites, S. chilense and S. pimpinellifolium species, showing the difficulty to find resistance sources to ToBRFV, contrary to other viruses.

All these sources had a high percentage of symptomless plants, these plants were however all positive by ELISA test, indicating that no total resistance or immunity was found.

For the controls, two plants were used S1 and S2, known to be susceptible to ToBRFV; control plants S2 were however resistant to TMV due to the presence of the Tm-2² gene.

The potential resistance/tolerance was assessed on the leaves of the plants; indeed, in view of the different shapes, colors, sizes of the fruits of these wild accessions, no ranking of the resistance at the fruit level was possible.

Table A reports the results obtained:

TABLE A
Assessment of ToBRFV resistance of the 4 potential
sources and two controls, by visual scoring and ELISA test

| | Nb of | 14 DPI | | | 28 DPI | | | ELISA | | |
|---|---|---|---|---|---|---|---|---|---|---|
| Source | plants | 9 | 5 | 1 | 9 | 5 | 1 | Nb tested | + | − |
| A | 44 | 44 | | | 30 | 14 | | 44 | 44 | |
| B | 8 | 7 | 1 | | 5 | 3 | | 8 | 8 | |
| C | 33 | 33 | | | 27 | 6 | | 33 | 33 | |
| D | 45 | 45 | | | 44 | 1 | | 45 | 45 | |
| S1 | 34 | | 5 | 29 | | 1 | 34 | Bulk | Bulk | |
| S2-TM2² | 32 | | 24 | 7 | | 13 | 19 | Bulk | Bulk | |

After 32 dpi, all plants were however positive by ELISA indicating that the resistance carried by these plants is not total (table A).

In view of the results obtained, the inventors have decided to focus the further work on source D, which shows the better level of leaf resistance at 28 dpi, although the aim of the study was to identify a source fruit resistance rather than a source of leaf resistance.

### F2 Mapping—HMC1*Source D—Artificial Test

Four F2 populations of 240 individuals each have been developed by crossing resistant wild Source D with a susceptible parent HMC1. HMC1 is a breeding line, indeterminate growth habit with red round fruits of around 100 g, it comprises the Tm-2² gene.

A visual scoring has been done at different dates: 7, 14 and 28 dpi (see table B above).

Table B shows the result of the F2 screening under artificial conditions: plants are scored on a 1 to 9 scale whereby plants with scores “1” or “3” will be considered as susceptible, plants with score “5” will be considered as intermediate resistant and plants with scores “7” or “9” as highly resistant with regard to foliar resistance.

The number of plants with a score of 1, 3, 5, 7 and 9 is reported in column 4-8. Column 9 (1=S) indicates the number of plants having a score of 1, 3, 5 or 7. Column 10 (9=R) indicates the number of plants having a score of 9. Columns 11 and 12 report the percentage of plants scored as ‘S’ (score of 1, 3, 5 or 7) and those scored as ‘R’ (score of 9).

TABLE B
Scoring of source D, and progeny thereof

| | | Nb of | | | | | | | | | |
|---|---|---|---|---|---|---|---|---|---|---|---|
| | | tested | | | | | | | | | |
| Pop | Name | plants | 1 | 3 | 5 | 7 | 9 | 1 = S | 9 = R | %1 = S | %9 = R |
| 1 | Source D | 12 | 0 | 0 | 0 | 0 | 12 | 0 | 12 | 0.00 | 100.00 |
| | F1: HMC1*Source D | 9 | 0 | 0 | 0 | 0 | 9 | 0 | 9 | 0.00 | 100.00 |
| | F2: HMC1*Source D | 238 | 31 | 14 | 35 | 22 | 136 | 102 | 136 | 42.86 | 57.14 |
| | BC1: HMC1* | 70 | 2 | 6 | 17 | 2 | 43 | 27 | 43 | 38.57 | 61.43 |
| | HMC1*source D | | | | | | | | | | |
| 2 | Source D | 11 | 0 | 0 | 0 | 0 | 11 | 0 | | 0.00 | 100.00 |
| | F1: HMC1*Source D | 11 | 0 | 0 | 1 | 0 | 10 | 1 | 10 | 9.09 | 90.91 |
| | F2: HMC1*Source D | 237 | 2 | 1 | 35 | 10 | 189 | 48 | 189 | 20.25 | 79.75 |
| | BC1: HMC1* | 71 | 3 | 11 | 31 | 1 | 25 | 46 | 25 | 64.79 | 35.21 |
| | HMC1*source D | | | | | | | | | | |
| 3 | Source D | 12 | 0 | 0 | 0 | 0 | 2 | 0 | 12 | 0.00 | 100.00 |
| | F1: HMC1*Source D | 6 | 0 | 0 | 0 | 0 | 6 | 0 | 6 | 0.00 | 100.00 |
| | F2: HMC1*Source D | 236 | 3 | 5 | 49 | 6 | 173 | 63 | 173 | 26.69 | 73.31 |
| | BC1: HMC1* | 64 | 3 | 7 | 17 | 0 | 37 | 27 | 37 | 42.19 | 57.81 |
| | HMC1*source D | | | | | | | | | | |
| 4 | Source D | 12 | 0 | 0 | 0 | 0 | | 0 | 12 | 0.00 | 100.00 |
| | F1: HMC1*Source D | 3 | 0 | 0 | 0 | 0 | 3 | 0 | 3 | 0.00 | 100.00 |
| | F2: HMC1*Source D | 235 | 1 | 10 | 37 | 14 | 173 | 62 | 173 | 26.38 | 73.62 |
| | BC1: HMC1* | 68 | 1 | 1 | 11 | 2 | 53 | 15 | 53 | 22.06 | 77.94 |
| | HMC1*source D | | | | | | | | | | |
| controls | HMC1 | 44 | 0 | 1 | 35 | 8 | 0 | 44 | 0 | 100.00 | 0.00 |
| | S1 | 32 | 32 | 0 | 0 | 0 | 0 | 32 | 0 | 100.00 | 0.00 |
| | S2 Tm-2² | 34 | 0 | 0 | 34 | 0 | 0 | 34 | 0 | 100.00 | 0.00 |

### QTL Analysis

DNA was extracted as detailed in example 1. For QTL analysis the inventors used different phenotypic variables/traits: Note 14 dpi, Note 21 dpi, Note 28 dpi, AUDPC.

The AUDPC (Area under the disease progress curve) was calculated by using the following formula:

$$AUDPC = {\sum }_{i = 1}^{n - 1} ( \frac{{y}_{i} + {y}_{i + 1}}{2} ) ( {t}_{i + 1} - {t}_{i} )$$

where “n” is the number of symptom assessments, “y” the symptom intensity (1 to 9) and “t” the time in dpi (days post inoculation).

The genotyping of the F2 population (based on Source D and HMC1) was done using a set of 169 SNPs. These SNPs were selected according the following:
Polymorphic/Allele frequency
SNPs placed evenly according to physical map distance

The QTL analysis was done using the QTL detection (ANOVA) model for biparental population in MAST—A Marker Assisted Selection Tool (proprietary software).

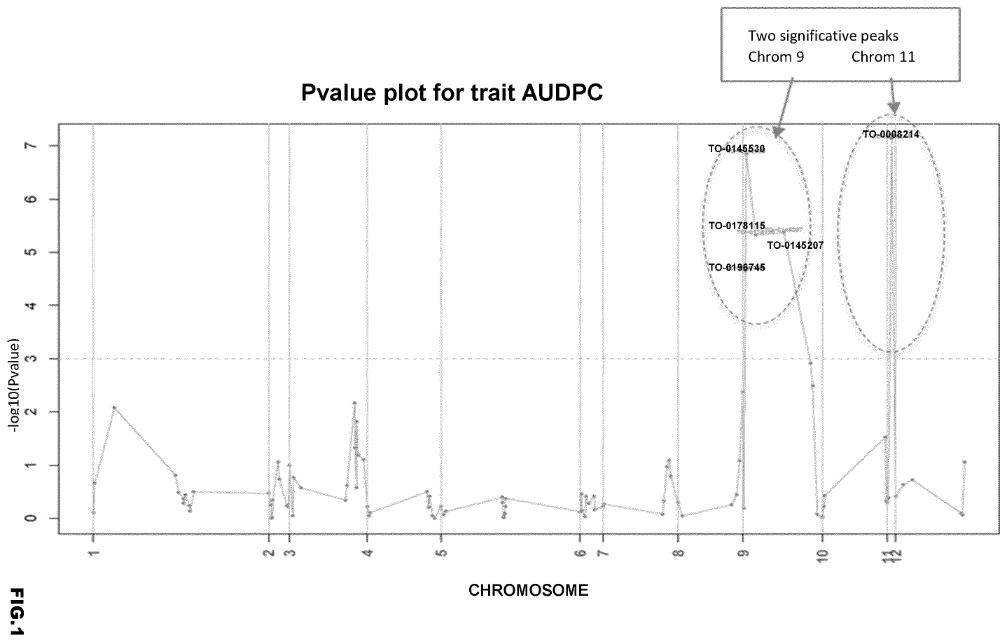

The mapping results (See FIG. 1 illustrating the Pvalue plot for the trait corresponding to AUDPC) revealed two QTL candidates associated with ToBRFV resistance, which are located on chromosome 9 (QTL9) and chromosome 11 (QTL11), the positive allele coming from Source D. Markers significantly linked with QTL9 and QTL11 associated to ToBRFV resistance and their position on the tomato genome are summarized in Table C.

| | Chromo- | Position | Pvalue | | |
|---|---|---|---|---|---|
| SNP | some | SL2.5 | Locus | LOD | R2.locus |
| TO-0196745 | 9 | 3 987 296 | 2.21E−05 | 4.66 | 0.04 |
| TO-0145530 | 9 | 4 654 259 | 1.41E−07 | 6.85 | 0.05 |
| TO-0178115 | 9 | 13 623 470 | 4.74E−06 | 5.32 | 0.04 |
| TO-0145207 | 9 | 40 039 587 | 4.26E−06 | 5.37 | 0.04 |
| TO-0008214 | 11 | 4 524 671 | 7.38E−08 | 7.13 | 0.05 |

Results showed that QTL9 responsible for ToBRFV resistance was located on chromosome 9, between position 3987296 and position 40039587, on the version SL2.50 of the tomato genome. This region of chromosome 9 is a region known to be of low recombination rate.

QTL11 responsible ToBRFV resistance is located on chromosome 11, at position 4524671, also on the basis of the version SL2.50 of the tomato genome.

### Example 3: QTL Mapping and Validation. Field Tests

### BC1F2 QTL Mapping and Validation

BC1F2 population between Source D and susceptible parent HMC1 has been developed using the SNPs described in Table C. 158 individual plants have been phenotyped under field inoculated condition in Jordan as described in Example 1.

The symptoms on the fruit were evaluated on a scale from 9 to 1:

9: no symptom—7: weak symptoms on few fruits—5: medium symptoms on some of fruits—3: medium symptoms on all fruits—1: strong symptoms.

Table D shows the result of the BC1F2 screening under field inoculated condition: plants are scored on a 1 to 9 scale whereby plants with 1 or 3 scores will be considered as susceptible, plants with 5 score will be considered as intermediate resistant and plants with 7 or 9 scores as highly resistant. No fruit evaluation was done for Source D because of the fruit size, namely small fruits due to the S. pimpinellifolium origin.

TABLE D
Fruit scoring of HMC1 and BC1F2 population

| Name | Generation | Nb of plants | Score 1 | Score 3 | Score 5 | Score 7 | Score 9 |
|---|---|---|---|---|---|---|---|
| HMC1 | S parent | 18 | 9 | 9 | 0 | 0 | 0 |
| HMC1 × Source D | BC1F2 | 158 | 44 | 38 | 43 | 18 | 15 |

DNA was extracted from the leaves as described in Example 1.

The BC1F2 population was genotyped with a subset of SNPs significantly linked to ToBRFV resistance in F2 mapping population. Marker trait association was done by cross ANOVA in MAST—A Marker Assisted Selection Tool (proprietary software).

The mapping result revealed that QTL9 is associated with fruit resistance to ToBRFV; i.e. the same region already associated with leaf resistance.

### BC3F2 QTL Mapping—Field Test

3 BC3F2 populations of around 140 individuals each between Source D and HMC1 have been evaluated under inoculated field condition. The protocol of inoculation and evaluation of the symptoms is identical to the protocol used for the BC1F2 and described in Example 1.

The scoring of these plants is detailed in Table E; the scale of scoring 1 to 9 is as detailed for the BC1F2.

TABLE E
Fruit scoring of HMC1 and BC3F2 populations

| | | Nb of | | | | | |
|---|---|---|---|---|---|---|---|
| Name | Generation | plants | Score 1 | Score 3 | Score 5 | Score 7 | Score 9 |
| HMC1 | S parent | 16 | 15 | 1 | 0 | 0 | 0 |
| Pop1 | BC3F2 | 141 | 16 | 46 | 29 | 35 | 15 |
| Pop2 | BC3F2 | 142 | 18 | 47 | 38 | 28 | 11 |
| Pop3 | BC3F2 | 143 | 22 | 51 | 29 | 23 | 18 |

DNA was extracted from the leaves as described in Example 1.

BC3F2 individuals were genotyped with a subset of polymorphic SNPs on chromosome 9 and 11 identified as linked to resistance to ToBRFV in F2 mapping population.

Genetic maps of chromosome 9 and 11 were built, using JoinMap software, for confirming the position and the order of the markers.

QTL detection was performed using MapQTL software and genetic maps done on these families.

QTL mapping results confirm the presence of a major QTL on chromosome 9 for fruit resistance to ToBRFV. Peak associated markers are described in table F.

TABLE F
Peak associated SNPs based on BC3F2 analysis

| marker | Position SL2.5 | p-value Locus | LOD | R2 Locus |
|---|---|---|---|---|
| TO-0123161 | 4 415 599 | 6.64E−28 | 27.18 | 0.26 |
| TO-0163151 | 8 147 577 | 9.35E−35 | 34.03 | 0.31 |
| TO-0145271 | 10 988 385 | 2.35E−33 | 32.63 | 0.30 |
| TO-0195825 | 13 23 303 | 6.44E−35 | 34.19 | 0.32 |
| TO-0163019 | 14 009 792 | 1.99E−35 | 34.70 | 0.31 |
| TO-0124744 | 16 017 044 | 1.05E−34 | 33.98 | 0.31 |
| TO-0197757 | 37 442 991 | 2.55E−36 | 35.59 | 0.33 |
| TO-0092241 | 37 443 206 | 3.54E−35 | 34.45 | 0.32 |
| TO-0107530 | 43 522 083 | 9.44E−35 | 34.03 | 0.31 |
| TO-0145125 | 44 428 308 | 8.79E−35 | 34.06 | 0.31 |
| TO-0146003 | 45 913 274 | 1.99E−35 | 34.70 | 0.31 |
| TO-0195624 | 46 235 227 | 3.98E−34 | 33.40 | 0.31 |
| TO-0195681 | 53 516 404 | 1.09E−34 | 33.96 | 0.31 |
| TO-0144921 | 58 380 538 | 4.20E−35 | 34.38 | 0.31 |
| TO-0124497 | 60 456 162 | 2.11E−34 | 33.68 | 0.31 |
| TO-0196929 | 62 242 444 | 2.51E−35 | 34.60 | 0.33 |
| TO-0124515 | 62 484 012 | 3.08E−32 | 31.51 | 0.30 |
| TO-0196109 | 63 417 056 | 7.13E−35 | 34.15 | 0.31 |
| TO-0109243 | 64 210 443 | 5.93E−29 | 28.23 | 0.27 |
| TO-0128512 | 66 029 732 | 2.08E−23 | 22.68 | 0.22 |
| TO-0144144 | 72 445 390 | 2.54E−26 | 25.59 | 0.24 |

### Example 4: Line Re-Sequencing and Identification of Unique SNPs

Six tomato lines including 3 sources of tolerance/resistance of ToBRFV, including the source D and the line used in WO2018/219941 (HAZTBRFVRES1), and 3 susceptible lines used as recurrent in mapping population were re-sequenced.

Seeds were sown and DNA was extracted from fresh leaves and whole genome sequencing was carried out.

Sequencing depth ordered was 20× minimum. Sequencing was done using Illumina NovaSeq 2×150 nt technology.

Reads were mapped on SL2.40 tomato reference genome and variant calling analysis was performed with samtools. To identify SNP from source D as unique as possible, SNP were filtered and the alleles found in source D were compared to the alleles of the SNPs identified in other lines of the project and 360 tomato genomes (“Genomic analyses provide insights into the history of tomato breeding”; Lin et al. Nature Genetics, 2014).

Based on that, 310 unique SNP from Source D were identified in an interval comprising QTL9 (from SL2.40ch09: 10 Mb to 55 Mb). To select the best ones some quality filters were applied (unique Blast on tomato genome and % AT in flanking sequence). A list of 101 SNPs was selected (see Table H). 14 SNPs (see table G) have been tested on a large panel of background and they have confirmed their capacity to track the presence of QTL9. Out of them 5 SNP (see table G) show very good results in term of specificity to source D.

TABLE G
List of 14 specific SNP from Source D resequencing in QTL9 interval.
The table gives the name of the 14 SNPs, the position in the SL2.50
genome, the sequence with the polymorphism in bracket, and the
susceptible and resistant alleles. The 5 SNPs showing very good
results are indicated by an asterisk.
The polymorphism is indicated within brackets. The column “S” reports
the susceptible allele, presence in the recurrent parent HMC, whereas
the column “R” indicated the resistant allele, as found in Source D.

| name | SL2.50 | Sequence | S | R |
|---|---|---|---|---|
| TO- | 11230042 | GTTGGGAGGCAGAAGGGTATAAGATTGGACACTGAAATTTTT | A | G |
| 0201220 | | GCTACTTACCGTAAGCTCATCAACATCGACTGTTAGAAACATC | | |
| * | | AGAGAAAGATATTTCTCAGACAGCTCACAGTAGAATGGAGAG | | |
| | | ATCATTCTACATGGACCACACCATGAAGCACTGAAATTTGCAA | | |
| | | TAACCTGCCAAAATTTCACTTGCGTAAGAA[A/G]GGTCCAAAA | | |
| | | TGCCAAAGCCAGCAAAAATGTAAAATTTTAAGCAAGCAAAAG | | |
| | | GCGAGGGATCCATATGGGGAAACTGCTGCACCATTCTGCTAT | | |
| | | TATCCTTCTCTTCGCATGTGTGCAAAGCATTAGAGTTAAAGTG | | |
| | | CCTTGAGTATTGTTTTTTAACGTCTTTTGAATTTTAGTGCAGCT | | |
| | | GGGCTAGAGGATTAAGTTAC (SEQ ID NO: 1) | | |
| TO- | 18086113 | TACTGTACCAATATTCAGTGCAAGGTATAGGTGTCCATTCATG | A | G |
| 0201221 | | AGGGCTGTCACCCATGAACACCGAAAATGCTGCTCACAGTAC | | |
| | | TTGCTGAGAGCTTCAGACGAACCATCCATTTGCAGATAATAT | | |
| | | CCAGTGCTCAAGATAGGGGTGTTTGTTGGAGCCGGTGAATTT | | |
| | | TTTCCAGAAGAGAGTATATGTTTGGGATCAC[A/G]GTCTGTGA | | |
| | | CAATGACCTCGGGATGTCCATGTAGCCTAATAATTTCAGAAG | | |
| | | CAAATGAAGAAGCCATAGTTTGAGCTGTGAAGGTAGAAGAAA | | |
| | | AGGCAATGATTGATTGCCCATATTTGAGTTACCAGTCAACCAA | | |
| | | CGTCAAGATTGTTACCTTCCCTTCGACCCCAAAAGGCAAGTA | | |
| | | ATGCATTGGTACCTCCTTAAACA (SEQ ID NO: 2) | | |
| TO- | 31451076 | TACTATCGAAGGGTAAAAATAAAAAACAAGTTGTTCAAATATC | G | A |
| 0201222 | | CATTAGAATGAAGAAAAGAAAAGCAAGTTAGGGGCTCTTGGA | | |
| | | CCGGGTGGTATCAATGTCTAAGCAATGGTTCTCCAGAGGCGT | | |
| | | CACACCATTCCAAGCCTTATTCAGCATGGAGAAAAATCCCTG | | |
| | | AAATCTAATGGCTCAAGTCTAGAAATAGCCT[G/A]GTCGATAG | | |
| | | ACACACAAGTAAAAATTACCTTTACCGTCTTCTACATCATTAG | | |
| | | CATTAGGCGTTCTCTGACCACTCCCTTCCTCTTCTCCTTTTGC | | |
| | | AAATGACGACAGAACATCTCGACTTCGAGGGAAGTTTTGGGT | | |
| | | GGGATTGGAGTAAAATCGTGTATTCACTCCCCATACTCAGCC | | |
| | | TCCAAGTCCTATTTAACCTGTG (SEQ ID NO: 3) | | |
| TO- | 29219882 | AAGTAGGAATACGGTCCATGGTCTGTGTTCATGGATTGAGAC | G | A |
| 0201223 | | ATAACTTACCCAATCACTTAAATGAACAACAGATGACCAGCG | | |
| | | CGGAGCATTGTTCAATCTATTGTCCGTATGTTTGATCATAACT | | |
| | | GAGGTTAGCAGTTAGTTACGGGAAATTTACGGATTGGATCAA | | |
| | | CTTAAAATTGTCATAAATTTTAGCACAAAAT[G/A]AATTAGGTT | | |
| | | CTCCATAACCCATGATATGATAGATAATTTAATATTCTCTCCAT | | |
| | | ATCCACTGAGTTTGCTAAAATTTGACCTCCGAGTGAAAAGTTA | | |
| | | TGCCTATTTTAGTGAAGGCCTATTGAGTAGACCCTAACGACA | | |
| | | GACCGTCTATTGAACAACGGCCCATCAGTCCAGGTCGTCGAT | | |
| | | TTCCGCGACAACAATTAGCT (SEQ ID NO: 4) | | |
| TO- | 27896293 | AACAACCCAATATTCTTAGGAAATGATCCTTAAAATGGTGCAT | C | A |
| 0201224 | | CTTGCTTAGACAACCAATGTAAGGGCTACTTGACTAGAGAGA | | |
| | | GAAGTCTCTGGGATGATTGAAGCTACCACAACATTTGGTTTA | | |
| | | GCTCCCCTCCATGTGTCTATTGATGATTTGACCGCTAGGGTC | | |
| | | ACAACTTGTGAGAGCAAGAAAGGGGAGAAAT[C/A]TGAGGCT | | |
| | | TTGTCTTTTAAAGACAAGGTAGCAAAGCAGAAGAAGGACATA | | |
| | | GACTATCCAAAGTCTACTGACTTCACTTCATTACTAGAGGCTG | | |
| | | CTAATGATTTAGAAACAATTGATACTTTAGAGATTACTTCGCC | | |
| | | TACCACCAGAGAGGTACATAGGGAGGATGCTAGAATTGATGT | | |
| | | ATTAGATGCTGAAACTGATGAGG (SEQ ID NO: 5) | | |
| TO- | 22586092 | CATTGCGAAATTGCATTTGTTCTCTTAATCAAAGTCTTGTAAC | G | A |
| 0201225 | | TTTGGGTGATTTAGTGTGTTCTCGGTGAACCGATCGGCAACT | | |
| | | CACCGACTACACATTTATGTCGCCGACTTGATTTTTCCCCCTC | | |
| | | CCCTCCGGGCTAGTATACTTGAACTGTAGGCGAATTGGGGA | | |
| | | GCCACTCGGCAGTTCACCAATTGGTTTTGGC[G/A]ATTACCAG | | |
| | | GATCTTCTTTTTCTCCACTTGTTCAGCTCGTTTTTCTCCCTTTT | | |
| | | TTCTAGAAGTGTCCTGCCTTGCTTATTAACTCATATACCTTAA | | |
| | | AATTAATGCTTTAACAATAGTTTGTAGAATAAAATAGGCATTTA | | |
| | | AGGACACTCAAACTACCAAAAAATATCCCTAAATGAGTAAAAT | | |
| | | CTTTGACTCATTATTTAT (SEQ ID NO: 6) | | |
| TO- | 23241474 | GTAATAGGGTCATAAAATACTTGGTGTGAATTTACCAAACACC | G | A |
| 0201226 | | CTAAGCATTGGCAGAAAACATATAGTGTTTGAACCCCTCAAA | | |
| | | GACGAGACCTAACAAATAGACCATCTGCGCATCAACGGACC | | |
| | | GTTTGTGGGGTCTCGTGGGTTGACACTTAGCTTTACATGGAG | | |
| | | CCTGACTAACAAACCTTGAAGATCCAACAGAT[G/A]GAGCAAG | | |
| | | AAGACAGGCCATCGGTTCAACCATTGGCTGTCGTTGCCTTTG | | |
| | | TTGGTGAACACCTCAAGGAAATTTTTAACCAAGATTTGGGCTC | | |
| | | TTTCTCTAGGGCCCCTTGTGGGTCAAAGGTGGGGTCGTACTA | | |
| | | AACAATTAAATTCCTTAACTATGTAATATAGGTTTAGGAATATC | | |
| | | TCATGCAGGTTCCAATAAAAAA (SEQ ID NO: 7) | | |
| TO- | 24571042 | TGAAATGTTTAAGGTTTAGATTGGTTGGTTCGCTCACATAGGA | A | C |
| 0201227 | | GGGTAAGTGTGGGTGCCAGCCCCAGCCCGGTTTTGGGTCGT | | |
| | | GACAATTGGACTTCCATCTGTGGTGATTTTAATACACCGATAT | | |
| | | GGGTTTTTCCCATGCTATTATCTTCACTTTAGTTTCATGAGTG | | |
| | | CAAGCATCCCCTAGTTGAAAGAGACCTTTC[C/A]ACATTGAAG | | |
| | | GCCATTTGATTTAAGGAAATGTTACAGCCAACATTCTGTATGT | | |
| | | ATCCATAATAATAACATATTTTACTCTACTCATGTCGGATGCT | | |
| | | CTTCCTTAATATATGCTTGCGCACCTATTCTTTCACAAGATCC | | |
| | | AACATATTATGACATGTCTGGCTTATAAGAGTAGTTTGTGTGC | | |
| | | CCCATTTTGGTGTTTTGTT (SEQ ID NO: 8) | | |
| TO- | 33866129 | GACTTGACTCTTAACTTTTAGTCAATTGAATTATGGATTCAAA | G | C |
| 0201228 | | GTTAATTATCTCATGTTTATGGATGATTTTAAGATTTTTGAGTG | | |
| | | TACCTTAAAGTGTGGGAATAAACTAGAAAACATAGGTATGTTG | | |
| | | CTAGGGTACTAGTACGAAATGAAGTGGGAAAAGATTGATGAA | | |
| | | TTGGTAACCCTGGCGCATTAAGAGGTGC[G/C]GGCCGCAAGT | | |
| | | GGGTAACATCAGATACTAGTGTAGGGTTTTTTGGCTGGTATG | | |
| | | GAGCCCCAGATTCCAAACTTCAGCGCCCCTAATGAGGCGCTT | | |
| | | TGGCTGGAACAGCGCCTTAGTCCCTTGCCCAGTTGAATTCCA | | |
| | | ACTTTTCTTGCTCATTTTTCAACTCTAAACCCCTAATTTCAACT | | |
| | | TGATTCTTTACCCAAACACA (SEQ ID NO: 9) | | |
| TO- | 39681557 | TATGAGAGAGAATTTTTTAAACCATCTAATCCATGAAAAGGCT | G | A |
| 0201229 | | TCACAGACATTAGATCAGTACAAGGACCGTCAATGGGGTTCG | | |
| | | TCAACCTAAAACTACAGACAGTGTGATATTTTTATTTTTTTCCA | | |
| | | TTGTTAGCTCGTCATCCTAAAAGTGTTTTGAGTGTGCCGTTAA | | |
| | | GATATATTGGTACTAAAAATTTCTCCGC[G/A]GGTACTATGTCT | | |
| | | CCCAAGAGGCAAGTGGTTTATACAAAAAGGGGCAAATTCAAG | | |
| | | TTAGTTGGCCCTTCTTCTTGGTTAATTGATGAAGACCTAGACT | | |
| | | AGAAGAGGGACCCAGCCTACATTCCTTCGGACATAAAAACAC | | |
| | | CACCAACAAAACCCTAAACTACTAAGAGCACCATTCTAATGA | | |
| | | GGACCGCACACTGTTCGGC (SEQ ID NO: 10) | | |
| TO- | 44268611 | AAAATTTAACCATCAAATCTTCATTAAACATAGACGTATGGGA | A | C |
| 0201230 | | TCTAATTCACCTTTGAGACCTCTTTCCTCAAAGGTTTTCTTCA | | |
| | | AAGTATTTTCAAAAGTTGAGTTTTCTTGTAGGTGCATTCATCC | | |
| | | GAATTTGGACCTCCCACAGTGGTTCAAAGTTCAAAAATTTGAT | | |
| | | GTCATTGGAGGAATAGGGAACCCCTTAG[C/A]GCATCTAAGG | | |
| | | TCCTATTGTTACAAACTAATGGGAGTTAGGAGTGATAAAGCTA | | |
| | | TATTGATGCAACTTTTTGGCAGAAGATTGAGCGGAGAAGCTT | | |
| | | TGGAACTGTTTAAATCTCACGAGACCAGACAATGAGATTGCT | | |
| | | GGAATGCCTCGGCTAATGAATTCATTGAGCGAGTTGCTTACA | | |
| | | ACGTAGAGATCGTCCCTGACT (SEQ ID NO: 11) | | |
| TO- | 50267259 | GAACTATCTAACTTCTTTCATCCTCCTTTTATGCGATAGAGTT | A | C |
| 0201231 | | CATCTTTATAAAAGTTTCTTTCTAACTCGTGCTTGCACGTATCT | | |
| * | | TTTAGATCATGCCTCCATGAAGATTTGTCTGAGCTAGTCGTTC | | |
| | | TAGGAGCAACGTTAAGGATCAAGAGGTGCCCAATGCACTAGA | | |
| | | AGTGCGACCCTAAGGAGAGGTCACTTCT[A/C]GGATCGGATA | | |
| | | ATGAGACAGATTGCGACCAACAAAGCTATGAAATAAAGAAAG | | |
| | | AATTGACAGGAGCTAGTTGATACTTCTAGCATTCGTGAGTACT | | |
| | | TAAGGATGAATTTTTCAAGCTTCAATAATTCAAGTGTGACTGA | | |
| | | GGATCCAGAATATTTTATAAAATAGTTGTAGAAAGTGATTGAG | | |
| | | GTCATGCGTGTTGCTTATG (SEQ ID NO: 12) | | |
| TO- | 50344933 | AGTTAGTGAGTTCTATAGTCTTTGGCAAGAGTTTTGGAGTATA | A | G |
| 0201232 | | ATATGATGGTAAAAGCTTGTAAAAATGGTTTCCGTACTTACAA | | |
| | | TAGGAGTTTAAGGGGTCCGGGTACGACTCCCCAAATACCAC | | |
| | | CAAGGGGTCCTTGAGGAGGACCCTAACACTGACCAAAGAGA | | |
| | | TTTCAAAACAACTTGGTTGATGGTTGCAATTC[A/G]TGACCAG | | |
| | | TAGGTTGGTCTACACCCCGTAAGTCAAGTCATGCACCACTCC | | |
| | | TACAAAGCTGATCCTCGGGTGCAAACCACAAAGGTGGTTGAC | | |
| | | GTACAGTAACTTCTCCTACAGTCCGTAGGTGGAGGGGTGTAG | | |
| | | GTGTTTCCAGTTTTAAGTAATTTATTTTAGTTAGGTGTTTGGTA | | |
| | | ATCGATTAGTTATACAATTACGG (SEQ ID NO: 13) | | |
| TO- | 53891374 | CATTAAGAGACTAAGGTCGTGGGGTAACAATCCATGGACCAG | A | G |
| 0201233 | | GACCACGACCTGTGACCTGGCCTAAGATCCGTGGATCTGGC | | |
| * | | AGTGGGTCGACCCTTAGAGGATTTTCTGGACCTTTTTGGGAA | | |
| | | GGGTTGTAGGTTGAAATCCACGGACCACCACTTACAGTCTGT | | |
| | | GCGTCACCCTAGTGGTAACTATGGATTGCACAT[G/A]TAGCTT | | |
| | | CTGGAATTAACATGCAACTTTTGGATGTTGGTTGGTCTTTTTA | | |
| | | GGATAAAAGGTGTTACATTATCTCCCCCTTGGAAATAATCTTC | | |
| | | CTCGAGTGAAGACTAAACTAGCTGAATATGAAGGAAAAGAGC | | |
| | | TTCCAACCCTACCACTAATTACTAGAAACTGATTTCTGACTGA | | |
| | | AATAAGTTCCAAGGACATGCAAA (SEQ ID NO: 14) | | |

TABLE H
List of 101 specific SNP from Source D resequencing in QTL9 interval.
Table H. List of 101 specific SNP from
LVSTBRFVRES2 resequencing in QTL9 interval

| | SL2.50 | | | |
|---|---|---|---|---|
| Name | position | Sequence | S | R |
| TO-0201220 ** | 11230042 | SEQ ID NO: 1 | A | G |
| | 12530620 | SEQ ID NO: 15 | C | A |
| | 12532418 | SEQ ID NO: 16 | G | A |
| | 12618950 | SEQ ID NO: 17 | G | A |
| | 12743421 | SEQ ID NO: 18 | A | G |
| | 13353638 | SEQ ID NO: 19 | C | A |
| | 13789540 | SEQ ID NO: 20 | G | A |
| | 13789892 | SEQ ID NO: 21 | A | C |
| | 13883222 | SEQ ID NO: 22 | G | A |
| | 13958935 | SEQ ID NO: 23 | T | A |
| | 13959395 | SEQ ID NO: 24 | A | G |
| | 14139566 | SEQ ID NO: 25 | G | A |
| | 14260504 | SEQ ID NO: 26 | G | A |
| | 14378558 | SEQ ID NO: 27 | A | G |
| | 14573359 | SEQ ID NO: 28 | C | A |
| | 14777274 | SEQ ID NO: 29 | G | A |
| | 14880923 | SEQ ID NO: 30 | C | G |
| | 15001952 | SEQ ID NO: 31 | C | A |
| | 15003643 | SEQ ID NO: 32 | G | C |
| | 15732459 | SEQ ID NO: 33 | A | G |
| | 16011851 | SEQ ID NO: 34 | A | G |
| | 16159620 | SEQ ID NO: 35 | G | A |
| | 16373133 | SEQ ID NO: 36 | G | A |
| | 16387459 | SEQ ID NO: 37 | G | C |
| | 16429793 | SEQ ID NO: 38 | A | G |
| | 16764120 | SEQ ID NO: 39 | G | A |
| | 17094866 | SEQ ID NO: 40 | A | G |
| | 17454360 | SEQ ID NO: 41 | A | G |
| | 17462277 | SEQ ID NO: 42 | G | A |
| TO-0201221 ** | 18086113 | SEQ ID NO: 2 | A | G |
| | 72450649 | SEQ ID NO: 43 | G | A |
| | 72458004 | SEQ ID NO: 44 | A | C |
| | 72458019 | SEQ ID NO: 45 | A | G |
| | 72472332 | SEQ ID NO: 46 | G | A |
| | 31986384 | SEQ ID NO: 47 | A | G |
| | 31817670 | SEQ ID NO: 48 | G | A |
| | 31730469 | SEQ ID NO: 49 | A | T |
| TO-0201222 * | 31451076 | SEQ ID NO: 3 | G | A |
| | 30874482 | SEQ ID NO: 50 | C | A |
| | 30074440 | SEQ ID NO: 51 | G | A |
| | 30063469 | SEQ ID NO: 52 | G | A |
| | 29606482 | SEQ ID NO: 53 | G | A |
| TO-0201223 * | 29219882 | SEQ ID NO: 4 | G | A |
| TO-0201224 * | 27896293 | SEQ ID NO: 5 | C | A |
| | 27571718 | SEQ ID NO: 54 | G | A |
| | 27011310 | SEQ ID NO: 55 | G | A |
| | 26965603 | SEQ ID NO: 56 | C | A |
| | 22052517 | SEQ ID NO: 57 | A | T |
| | 22490493 | SEQ ID NO: 58 | G | A |
| | 22529379 | SEQ ID NO: 59 | A | G |
| TO-0201225 * | 22586092 | SEQ ID NO: 6 | G | A |
| | 23217240 | SEQ ID NO: 60 | G | A |
| TO-0201226 * | 23241474 | SEQ ID NO: 7 | G | A |
| | 23472188 | SEQ ID NO: 61 | G | A |
| | 23561322 | SEQ ID NO: 62 | G | C |
| | 24076201 | SEQ ID NO: 63 | A | G |
| | 24076481 | SEQ ID NO: 64 | A | G |
| | 24457122 | SEQ ID NO: 65 | G | A |
| TO-0201227 * | 24571042 | SEQ ID NO: 8 | A | C |
| | 24582850 | SEQ ID NO: 66 | G | A |
| | 25081514 | SEQ ID NO: 67 | C | A |
| | 25097623 | SEQ ID NO: 68 | G | A |
| | 25470743 | SEQ ID NO: 69 | G | A |
| TO-0201228 * | 33866129 | SEQ ID NO: 9 | G | C |
| | 34989750 | SEQ ID NO: 70 | A | C |
| | 37148403 | SEQ ID NO: 71 | G | C |
| | 37299973 | SEQ ID NO: 72 | G | A |
| | 37299973 | SEQ ID NO: 73 | G | A |
| | 37357370 | SEQ ID NO: 74 | C | A |
| | 38444425 | SEQ ID NO: 75 | A | C |
| | 38810552 | SEQ ID NO: 76 | A | C |
| | 38845652 | SEQ ID NO: 77 | C | A |
| | 39490875 | SEQ ID NO: 78 | A | T |
| TO-0201229 ** | 39681557 | SEQ ID NO: 10 | G | A |
| | 40035805 | SEQ ID NO: 79 | T | A |
| | 40035805 | SEQ ID NO: 80 | T | A |
| | 40924871 | SEQ ID NO: 81 | C | A |
| | 40924933 | SEQ ID NO: 82 | C | G |
| | 41038034 | SEQ ID NO: 83 | C | A |
| | 41817777 | SEQ ID NO: 84 | A | C |
| | 41982741 | SEQ ID NO: 85 | G | A |
| | 42546269 | SEQ ID NO: 86 | A | T |
| | 42832741 | SEQ ID NO: 87 | A | G |
| | 43586119 | SEQ ID NO: 88 | A | C |
| | 43989356 | SEQ ID NO: 89 | A | G |
| TO-0201230 * | 44268611 | SEQ ID NO: 11 | A | C |
| | 45846023 | SEQ ID NO: 90 | G | A |
| | 47213666 | SEQ ID NO: 91 | A | T |
| | 47280041 | SEQ ID NO: 92 | G | A |
| | 48841288 | SEQ ID NO: 93 | G | A |
| | 48952734 | SEQ ID NO: 94 | G | A |
| | 48969738 | SEQ ID NO: 95 | G | A |
| | 50116569 | SEQ ID NO: 96 | G | C |
| TO-0201231 ** | 50267259 | SEQ ID NO: 12 | A | C |
| | 50267370 | SEQ ID NO: 97 | A | G |
| TO-0201232 * | 50344933 | SEQ ID NO: 13 | A | G |
| | 51722033 | SEQ ID NO: 98 | C | A |
| | 52713471 | SEQ ID NO: 99 | A | T |
| TO-0201233 ** | 53891374 | SEQ ID NO: 14 | A | G |
| | 54227184 | SEQ ID NO: 100 | A | G |
| | 54305707 | SEQ ID NO: 101 | G | A |

The table H gives the position in the SL2.50 genome, the SEQ ID number of the sequence in the sequence listing, and the susceptible and resistant alleles of the 101 SNPs identified by the inventors. The 14 SNPs and 5 SNPs mentioned above showing very good results are indicated by one or two asterisks respectively.

These SNPs, thus allow the discrimination between plants having the QTL9 as described in the present invention, and those derived from HAZTBRFVRES1 described in WO2018/219941 having a different QTL on chromosome 9 providing tolerance to ToBRFV. The QTL disclosed in the present invention is thus clearly different from a sequence point of view from the QTL disclosed in WO2018/219941.

### Example 5: Characterization of the New QTL9 in Field Tests

Two different trials were conducted in Jordan, in order to confirm that, in addition to the differences in sequences of the QTL9 of the present invention and of the QTL mentioned in WO2018/219941, these different sequences moreover confer a different type of resistance to ToBRFV.

A first trial T1 (372 plants) was conducted in summer, in one tunnel, with different elite lines (checks), the controls S1 and S2 mentioned in Examples 2-4 and plants comprising the QTLs on chromosomes 9 and 11 mentioned in WO2018/219941.

A second trial T2 (1165 plants) was conducted during the following winter, with two tunnels, comprising the same elite lines (checks) and controls as for the previous trial T1, and BC3F2 issued from source D, mentioned in example 4.

In this example, the type/origin of the sequences found at the loci of QTL9 and QTL11 on chromosomes 6 and 9 of the tested plants is defined as follows:
Sequences found in the elite lines at the loci of QTL9 and QTL11, and by extension “allele” of these loci in the elite lines, are coded as “Re”;
Sequences found in the susceptible lines S1 and S2 at the loci of QTL9 and QTL11 (“allele” of these loci in the susceptible lines) are coded as “S”;
Sequences found in the tolerant/resistant plants derived from HAZTBRFVRES1 described in WO2018/219941 at the loci of QTL9 and QTL11 (“allele” of these loci in HAZTBRFVRES1) are coded as “Rh”;
Sequences found in the resistant plants BC3F2 issued from source D at the loci of QTL9 and QTL11 (“allele” of these loci in the plants of the invention) are coded as “Rd”.

The plants were inoculated twice, a first time one week after planting, and the second time after two weeks, as described in example 1.

The plants were then scored, with regard to fruit and leaves symptoms, according to the scale mentioned in example 1.4. Insofar as the leaves symptoms were evaluated at the adult stage (plants bearing red fruits), the leaves symptoms were however difficult to assess in view of the rare young leaves.

### Statistical Analyses of the Results.

Data were analyzed, using a mixed model in order to determine the level of resistance imparted by the “allele” found at the QTL9 locus of the plants, taking account of the other potential effects, namely the “allele” found at the QTL11 locus, the genotype of the plants, and the effect of the tunnel. The mixed model was as follows:

Score=μ+(Genotype)random+Tunnel+QTL9+QTL11+ε

wherein the score (fruit resistance or leaf resistance) is the observed variable, μ is the mean value of the trait, ε is the residual error and wherein (genotype) random, Tunnel, QTL9 and QTL11 are the effects.

The tunnel effect was estimated thanks to the checks present in all tunnels.

The genotype effect was treated as a random effect to catch the variability of the population, from which the tested varieties are coming.

For each genotype of the QTL9 locus, and for each trait (fruit symptoms and leaves symptoms) the inventors extracted the adjusted values in order to get a better estimation of the potential of each genotype, independently of the other effects.

For example, for the genotype corresponding to Rd/Rd, the adjusted value is:

μ+QTL9[RdRd]+average(Genotype)+average(Tunnel)+average(QTL11)

wherein μ is the estimated mean value of the trait (fruit symptoms or leaves symptoms), QTL9[Rd/Rd] is the estimated effect of the genotype RdRd for QTL9 and average (Genotype), average (Tunnel) and average (QTL11) are the average of the corresponding estimated effects.

A Tukey's test was used to perform multiple comparisons between the adjusted values

### Results:

### QTL9 Effect on Fruits Symptoms:

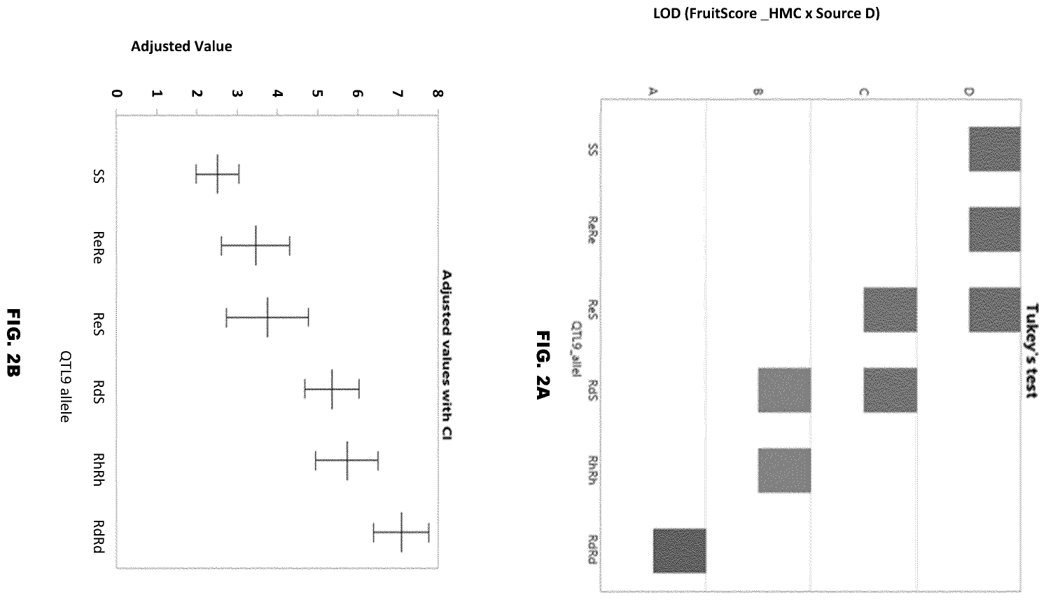

For the trait corresponding to fruit symptoms, the adjusted value of the different tested genotypes regarding the QTL9 locus, was calculated using the mixed model detailed above. The results are reported in Table I and are illustrated on FIGS. 2A and 2B.

TABLE I
QTL9 effect on fruit symptoms

| QTL9_allele | Fruit symptoms adjusted | Group | N |
|---|---|---|---|
| RdRd | 7.0853 | A | 203 |
| RhRh | 5.73088 | B | 78 |
| RdS | 5.3609 | BC | 214 |
| ReS | 3.75473 | CD | 56 |
| ReRe | 3.46006 | D | 132 |
| SS | 2.51856 | D | 560 |

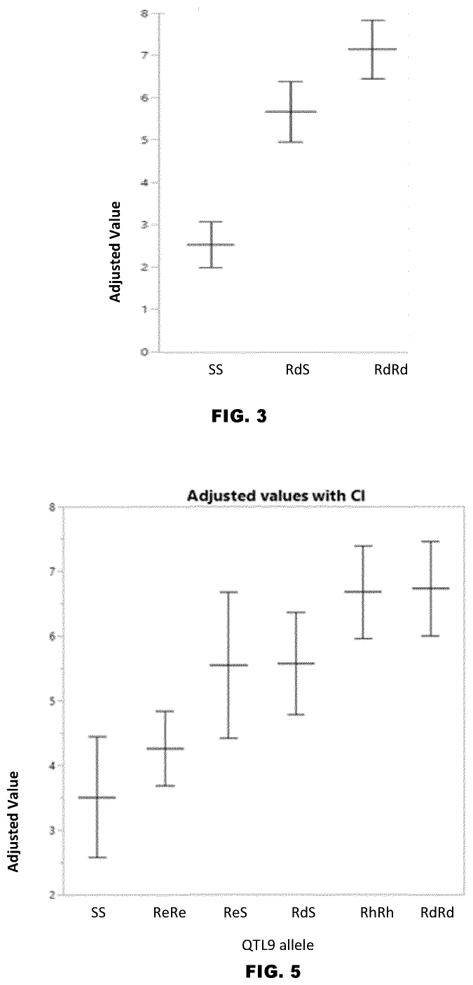

N = number of corresponding plants
Group: group allocated according to the Tukey’s test
Rd, Rh, Re and S correspond to the genotype of the plant, for the sequences at the QTL9 locus.
From these results, it can be deduced that the presence of QTL9 as defined in the present invention (corresponding to Rd genotype in this example) provides a level of resistance which is significantly different and significantly higher than the level of resistance provided by the QTL2 as defined in WO2018/219941 (corresponding to Rh genotype in this example).
Moreover, the analysis of the effect of the alleles at QTL9 on the fruit resistance shows a significant additive effect of the Rd alleles, as shown below (extract of table I) and illustrated in FIG. 3.

| QTL9_allele | Adjusted Values | Group | N |
|---|---|---|---|
| RdRd | 7.0853 | A | 203 |
| RdS | 5.3609 | BC | 214 |
| SS | 2.51856 | D | 560 |

The fruit score repartition has also been determined by QTL9 genotype. The results are illustrated on FIG. 4.

As shown by these results, the QTL9 according to the invention, when present at the homozygous state (genotype RdRd in this example), gives more than 80% of plants having a fruit score of 5, 7 or 9 (respectively 48, 61 and 59 plants out of 208). Around 60% of plants (120 out of 208) have a score of 7 or 9, i.e. almost no symptoms on fruits, after two rounds of infection.

These results also confirm that the QTL9 according to the invention, when present at the heterozygous state (genotype RdS in this example), gives around 50% of plants having a fruit score of 5, 7 or 9 (respectively 63, 36 and 13 plants out of 225). Around 22% of plants (49 out of 225) have a score of 7 or 9, i.e. almost no symptoms on fruits, after two rounds of infection.

By comparison, for the RhRh genotype, also around 80% of the plants have a fruit score of 5, 7 or 9, but less than 35% have a score of 7 or 9 (respectively 30 and 1 out of 89), i.e. almost no symptoms on fruits, after two rounds of infection.

### QTL9 Effect on Leaves Symptoms:

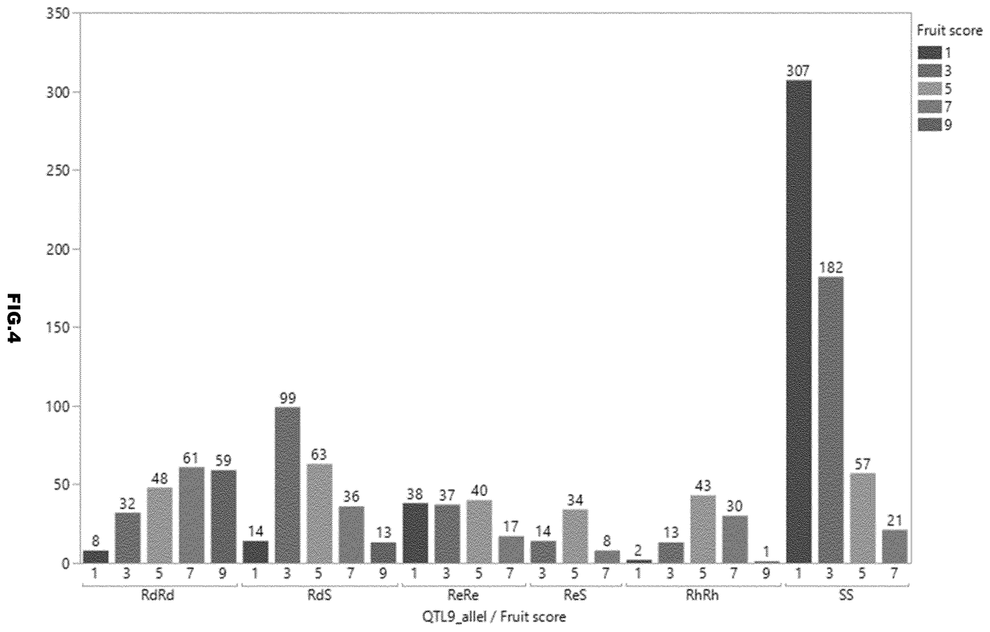

For the trait corresponding to leaves symptoms, the adjusted value of the different tested genotypes regarding the QTL9 locus, was calculated using the mixed model detailed above. The results are reported in Table J and are illustrated on FIG. 5.

TABLE J
QTL9 effect on leaves symptoms

| QTL9_allele | Leave symptoms adjusted | Group | N |
|---|---|---|---|
| RdRd | 6.73072 | A | 198 |
| RdS | 6.67866 | A | 199 |
| RhRh | 5.57343 | B | 81 |
| ReS | 5.54822 | ABC | 51 |
| SS | 4.26684 | CD | 562 |
| ReRe | 3.51093 | D | 148 |

N = number of corresponding plants
Group: group allocated according to the Tukey’s test
Rd, Rh, Re and S correspond to the genotype of the plant, for the sequences at the QTL9 locus.
From these results, it can be deduced that the presence of QTL9 as defined in the present invention (corresponding to Rd genotype in this example) provides a level of leaves resistance which is significantly different and significantly higher than the level of leaves resistance provided by the QTL2 as defined in WO2018/219941 (corresponding to Rh genotype in this example).
Moreover, the results also show that the presence of the QTL9 heterozygously is sufficient to provide a high level of leaves resistance (adjusted value for the genotypes RdRd et RdS are statistically identical).

### Example 6: Line Re-Sequencing and Identification of Unique SNPs on Chromosome 11

The same experiments disclosed in example 4 for QTL on chromosome 9 were applied to the QTL on chromosome 11 identified by the inventors. The SNP analysis was conducted on the same population of plants.

The SNPs which are informative regarding the presence of the QTL11 according to the invention are reported in table K below (SEQ ID NO:102-111), as well as their position in the SL2.50 version of the genome and the susceptible and resistant allele.

The inventors have then conducted a further search in order to identify additional markers informative regarding the presence of the introgressed sequences conferring ToBRFV resistance. These markers are very specific to the introgressed sequences from Source D conferring ToBRFV resistance.

TABLE K
Markers corresponding to QTL11

| | Position on | | | |
|---|---|---|---|---|
| Marker name | SL2.50 | Sequence | S | R |
| TO- | 8744810 | GTTACTTTAATATTTAATTGTATTATAATAAATTCTATATACTA | A | G |
| 0201237 | | AGGGATGCAGTTATTTTATAATTTTGAGAAAATACATGGCAT | | |
| | | ATTCGTTCATCTTACTTGTGACCTTTTTTTTAATAGATTATAG | | |
| | | TACATTCCTTATTAAATGGGTTAAAGCTATGATCTGGAAATC | | |
| | | TCCATGCTTAACGTCTGTATGTTTTCAAT[A/G]CTATGGTCAA | | |
| | | ACAATTTGCTCATATAATTTGTGTACGTGTTATGAATTTGGG | | |
| | | ATTTTTCATATGAGTAAAATAGTTTTTAAAAAAAATTCTGTTA | | |
| | | TGTCCTTGTGGGCATAGGTTCAACCTTGAAGTAAATTTAGG | | |
| | | GATCATAATAATGGGTGTTGTTAATTTTGAAATCTTATTATG | | |
| | | GTCATTTGTTTGAGTAGATTGC (SEQ ID NO: 102) | | |
| | 8769601 | AAAACAATTTCAATTATGTAGAATCATTTTTCGGCCACATTAA | C | G |
| | | ATCATATTTTTATGTCATTGAACCTTGAGAAAATACTCATCCA | | |
| | | TATCAATTATTAAAAGTTTAATTTATCTATACGCTATATTTCA | | |
| | | ATTAACAATAACGACATGCACGAATTAAATTTTTAACAAATG | | |
| | | ACATAAATGTGTCTTGTCTCAAAATTCGA[C/G]GAGATCTTTC | | |
| | | GTTTTCTTTAATATTCATGAAAAAACATTGATCCAAAGTGGA | | |
| | | AAGTTAAGGATGAGTTGGACATTTTTCACTTAAAATAATGTA | | |
| | | ACGTGACTCCCTCCATTTTCCCCAAACGTAAGTCCAAGTCA | | |
| | | ACTTGAGACACCATATTATTATAATAAAAATAGAACAAACAC | | |
| | | AAATCCATAAATTTACAACTTGA (SEQ ID NO: 107) | | |
| | 8922843 | TTCCAATGGGAGAAACTTGAGAGGAAGTGAAAATGTGGTGG | G | A |
| | | AGATAAACATGGTTATGGAGAGACATTTGAGGCAAAATATG | | |
| | | AGAGAAATATTTGAGAGATTTTAGGGGATTAAATAAGTGAGA | | |
| | | GAGAATATTAATTATTAATTATTAATAAAGAAATGCCAAATTT | | |
| | | ATTTACACAAAACGTGTAAAATGGACATTTTGT[G/A]TCAATC | | |
| | | TTTTAAAATCTGGCTAATGGATGCTATAAATTGAAGTCTGAG | | |
| | | TTGACATGCTATGTCATTTTCCTCAATGGGCTGTTGGAGAA | | |
| | | GAGAATTGGGTTCAATATGGGAGACAAAACTTGGCTAGAAT | | |
| | | TGAGTTAATTACTAAGTTTGGTTAAAAATTAAATAAGATAAAT | | |
| | | AAATTAATGTTACTTAATTATGGGAAA (SEQ ID NO: 108) | | |
| TO- | 8972905 | TGCAACTTCTATTCTTCCTTCCGCAGGTGGGAGCTGTGTAT | T | A |
| 0201238 | | TTTATTTATCTTGTCTAGTTGTACTCTTATTAGCTTTTGTACT | | |
| | | TGTTTAACTAGATCCATGAGAGTTGATAAATTACTTTACAAG | | |
| | | TTTTAAGTAAATGTGTGTCTAAAGATATTATTTATAAAATCAT | | |
| | | ATTATTGCAATTACTTCTGTTTCCTTAAAAA[T/A]TTTCACATG | | |
| | | TTTAATTATTGGGATACGAGAGTTCTCTTACTTAGGTGTTAG | | |
| | | AGTGGTGCCTGGACGGCTCGGTGGGTTGGATCGTGACATA | | |
| | | ATATATGAATTTGTCAAAAAAATTTACATTATTTTTGTGATCG | | |
| | | CTATTTCAATATAATATACAGTAAGCATGCATATATGTACAAT | | |
| | | GGATATATTTTTGTCATGCTCTA (SEQ ID NO: 103) | | |
| | 9225114 | CGTGGACGGTACTCGACGACCATTGTTGGTCCCTAAGCGA | C | A |
| | | ACCCTTATCCTGGCTGACTACAAGCGGAAGACTAACTTAAG | | |
| | | CATACCAAAAATCTTAAAACTGAAATGAACAACTTAGAATAA | | |
| | | AATAATCTATTTAACTAGTCATTAAATAGGCAACTTGAGTCTT | | |
| | | TAAAACATTTAATAAAATAAATTATTAATAAAAA[C/A]TTCTCA | | |
| | | ACTAGACTATCTATGGAGCCTCTAACTGAAAAAGATGGAAA | | |
| | | TCGGGACACCACGACATCTTGATTAAACTGAAAAGTAAATA | | |
| | | AGGTCCTCCGGAAACAAGGAGGTTCAACGACTAACTCGAA | | |
| | | CTCTAGGATGTATTAATAAAGCTCTTGTTAATGATCCCGAAG | | |
| | | ACCTGTGTTTGCATCATCAATTATGCAACC (SEQ ID NO: 109) | | |
| | 9284958 | AGCTTCCTTACTTATTTAAATGTGAAAACATATCATTTGGGG | G | A |
| | | ATACTTAGTTCCCCTATACTTTTGAAGAAAAGAACTTGAACT | | |
| | | TAACTCTTTACTCTTTACTTAGCTTGAAACTTGACTCTTAAG | | |
| | | GAATATTTAGTTCCCTTATATTTTTTGAGAAATGCACTCACTC | | |
| | | TTTGCTCTTTGCTCTATTTGAAACTTAACTC[G/A]TAGGGAAT | | |
| | | ACTTAATTCCCTTATAAATTTTGAGTTTTAAACTCAAATCATT | | |
| | | ACTCTTTTCTCAACTTGTAGTTTAAGCCTTAAAACAAAGTTTA | | |
| | | AAATGTTGTTTAAGACTCTTTAAAATAGTAAGAACTTACTTTG | | |
| | | ACTTTCTTTTTATTTGTAAGACTTGATTCTTAAATTCTTTGGC | | |
| | | TTTGATATTAACTTTCCTTG (SEQ ID NO: 110) | | |
| TO- | 9290095 | TGCGAGGCACTCGCCTTGATGGTTCTTACTACACTTGGGGC | C | A |
| 0201239 | | AAGTGGGGTAAGTCTTGGTGCTTGAAACACTTCCCTAAGAC | | |
| | | TTAGATTCTGGTGCCCTAACCTTCTGATCATACATGTTCTTA | | |
| | | GAGGATGGAATACTAGTTATCATAGTTCTGCTATGGGGTTG | | |
| | | AAAACTTTTCCTGACTCTACGAGCGATTTCCACCA[C/A]CAA | | |
| | | ATTTCTGCTAAGAGTAGTCATAGTTCCCAGTCCTAACCTCTT | | |
| | | ATTCTCCTTGGCTTGTTTCCTAAGCTTATCACCCTCAACCTG | | |
| | | CTGATCATGAGTCATAAGCTTAAGGATGTTCATATCTCGTAG | | |
| | | TAACATAGAACTTCTGCACTCAGTTTTCACCAAATTTTGACT | | |
| | | CCGTACAAGAAATTATTCATCTGAGCCCT (SEQ ID NO: 104) | | |
| | 9356623 | TATATCAGAATGATCAAATAATCGCGTCTTTAGAATTTTATA | A | T |
| | | GGCATCATTGTTGCTGCTAAGATATTTTGACCTAATGATCAA | | |
| | | ATAATCGCGTCTTTAGAATTTTATAGGCATCATTGTTGCTGC | | |
| | | TAAGATATTTTGATAGGTCAAATGATGTAATAGGGTTTTTGT | | |
| | | AGAGGCCAGATCGTAAATTGGTGAAGGCAAAA[A/T]CAGAAA | | |
| | | CTGTACAATATGATTTTGATAGAGATCAGTTCTTTTTCTTGA | | |
| | | GATGTTATGGAGGATTCATCTATTAGGATCTAGTACAAGGA | | |
| | | AAATTTGTTATGTATTGAAGTATTTGTTGAGAACATTTTTGAA | | |
| | | TTACCTTGTGTAAGTTTAAGATTATGAAGAATTCTTTTCTTTT | | |
| | | TCTTTCTACTAGATGGGTAAGCACG (SEQ ID NO: 111) | | |
| TO- | 9461743 | AGTACAAATTGCAACTTTTTCAATGTTAACGTCTGCTTTTTTA | C | A |
| 0201240 | | GTGTATAAAAAAGAAGGATTCATGAACTCCTCAGTCATATTC | | |
| | | TATTTTAAAATACTTAAACATCTTTTTTAATTATGTCTCAAGC | | |
| | | ATGTCAAACATCTAATGCAAAAAAGACTTACGTTTGTCGTTG | | |
| | | TGGTAATTTAGCTATTTTGAGGATTTCACA[C/A]ACCGATACG | | |
| | | AACCCAGATCGATAATTATTTAATTGTGCAATTGGTGCATTG | | |
| | | ATCTTTTTTTAAGTGGCTTGATTATGATTCATCAACGAGCAA | | |
| | | CCCATCAAAATTGAGTCTCGGATTCGAGACATCAAATTTTCA | | |
| | | AAGCGTTATCAAATTTCAAATATTCTTTATCTCCTTATTTTAT | | |
| | | CTCATGTACAACTTGATTGACT (SEQ ID NO: 105) | | |
| TO- | 9550430 | TCATCGTAGTCAGTAAGATTTTACCAAGGAATTAAAATTTAT | C | A |
| 0201241 | | TAAGTATTAAGCTCATTACATTAGAATTAGCACCAAGATTTC | | |
| | | CAAAAAGAACTAAATTGGAATTAGCAAAAATCTGAAATTTGA | | |
| | | AGAGTTAAGTTAAGTATGAGTTTTGAGTCAACTTCAAAGGAC | | |
| | | CATAAATATTAGAACACGATGAGTTAGATGTG[C/A]TACAAG | | |
| | | ATACCATAGGGAAGATATTCGAATAACCTTTGCAAAGCCTTT | | |
| | | GAGTTTACTAAGTTTCAAGTTCATACAAGTGAGATATGACCT | | |
| | | TTTGAAGTTAGGTTCTCCAGTTAAGCAAAGTTAGCCAAAAAT | | |
| | | AGTGAAGGTATTTTGGTCCTTCCCTTACCCAAACAGATTTAA | | |
| | | TTCGTTTTAGTCATATTTTAGGGTTA (SEQ ID NO: 106) | | |
| SL2.50ch11_ | 9684449 | AGTGATATTGTCTTCAGCTATAGAATATTATCCAGCCCCTCT | C | CT |
| 9684449 | | AGCTTTGGTCGATTTTTTTTTATAACAAATTTTATCCAAGCTA | | |
| | | TCATTGTTTTTTTTTTTTTTTCAATTTACAGCAATTGTTTCCC | | |
| | | GACTAGCCTATCAATGTTAGAG[C/CT]TTTTTTTTGGTTTGAA | | |
| | | CATCGTTGTCTTGAGAAGCTTAATCCATGTTCACTGTAGGC | | |
| | | ATTGTTATTTAATTTTCAATTGAGTTTGTTAATCTGCCTATAG | | |
| | | ATATTAATTTATTTCCTCTTCTTTCGATGCATATGATCAATCC | | |
| | | AGTCTAT (SEQ ID No: 112) | | |
| SL2.50ch11_ | 9779896 | ACAATGACATGACATCAAAAATAATTTAATTTATCATGTCAAA | A | AT |
| 9779896 | | TTTGTTTAAAAATAAATTGTAATCGAGTGATTTTAAAGAATAA | | |
| | | AGATGTTAAGTTGAGTGATTTTAAAGACACAAACTAAAATTT | | |
| | | AGTGACTTTACAAGATAATTTC[A/AT]TATAGTTCATTGACCT | | |
| | | TTTAAGATATTAACTCAAAAAATCAATAAGTAGTCACACACA | | |
| | | AAAGGTGATTGCAAAGTAAATTTCAATTTTTAGCATAAGCTT | | |
| | | AGTGGGGAATACCAATGACGAATCTAAAAGTATCAATACAA | | |
| | | AAAATAGAA (SEQ ID No: 113) | | |
| SL2.50ch11_ | 9823405 | AAATGTTAATTTTTTTATTTATAATAAATATTAATATGTCTTAG | T | C |
| 9823405 | | ACTTGAACTTTTAAGTTCATTAAAGATAGTTAAATATCGTATT | | |
| | | TGATATACTTAACGAAAAAGCTAAAACGTAAAAGAAAATATA | | |
| | | ATAAGTTTATATCTTCTTCTT[T/C]TTTTTTTAAATTTGGATTT | | |
| | | GTGATAGTAGGGCTTTGAGAGAAAATTCTTTTTATAATTTTTT | | |
| | | TTTTCATATTCAAGGCTCGAACCTAAGATCTATGATTAAGGA | | |
| | | AGAAGTAGCCTTTCCACTATACAATACACGTTCATAATGAGT | | |
| | | TAAT (SEQ ID No: 114) | | |
| SL2.50ch11_ | 9924232 | GCAAAAAAATAAAAAATAAAAAAACGCTAGACAGATTGATGA | G | GT |
| 9924232 | | CTATTTATGCTCATTTCAAAACATAACTAATTTGTATTTAATT | | |
| | | TTTTTTTACCTTTTTTGATTATTTTTTTTGCTGTATGATTTTCT | | |
| | | CGGTTCTATTATTAGTTATTT[G/GT]TTTTTTTAAATATTAATC | | |
| | | ATAATAACATAAATACATAATTAATCCCTTAACAAAATAAAAT | | |
| | | AATGACATTTTTAATAAACAATAAAATTGTTGCTAAGAAGTC | | |
| | | CCACTAAAAGCTTAAATATTGTTGTTTTGAAAAAAATTTCCC | | |
| | | CAAGG (SEQ ID No: 115) | | |

### Example 7: Genetic Modification of Tomato Seeds by Ethyl Methane Sulfonate (EMS)

Seeds of a tomato varieties are to be treated with EMS by submergence of approximately 2000 seeds per variety into an aerated solution of either 0.5% (w/v) or 0.7% EMS for 24 hours at room temperature.

Approximately 1500 treated seeds per variety per EMS dose are germinated and the resulting plants are grown, preferably in a greenhouse, for example, from May to September, to produce seeds.

Following maturation, M2 seeds are harvested and bulked in one pool per variety per treatment. The resulting pools of M2 seeds are used as starting material to identify the individual M2 seeds and the plants with a resistance to Tomato Brown Rugose Fruit virus.

### Example 8: Resistance to Stemphylium

WO2020/018783 discloses a genetic region on tomato chromosome 11 that comprises a Stemphylium resistance allele from S. pimpinellifolium, and allegedly also comprises a TBRFV resistance allele, which are both so closely linked that they are characterized by the very same markers and thus introgressed simultaneously. In order to confirm that the QTL on chromosome 11 identified by the present inventors is different from the resistance allele disclosed in WO2020/018783, the present inventors have tested the plants according to the invention for resistance to Stemphylium.

The protocol for Stemphylium resistance is as disclosed in Example 1.5.

### Results:

More than 157 different plants were tested, from 5 different genotypes, with at least 18 repetitions per genotype.

The tested genotypes/cultivars were:
One Stemphylium resistant control (R)
One Stemphylium intermediate resistant control (IR)
One Stemphylium susceptible control (S)
Source D
BC5F3 HMC1*Source D

The results of Stemphylium resistance are reported in Table L below.

TABLE L
Stemphylium resistance.

| | | | | | Nb of |
|---|---|---|---|---|---|
| CULTIVAR/ | QTL11 | Mean of | Vari- | Inter- | tested |
| FAMILY | source D | symptoms | ance | pretation | plants |
| R control | NT | 9 | 0 | R | 24 |
| IR control | NT | 8.9 | 0.2 | R | 18 |
| Source D | Present | 1.2 | 1.0 | S | 35 |
| S control | NT | 1 | 0 | S | 42 |
| BC5F3 | Present | 1 | 0 | S | 35 |
| HMC1*Source D | | | | | |

“Mean” indicates the mean score of all plants from the same family, according to the symptom evaluation detailed in 1.5. Interpretation indicates whether the cultivar is to be considered as resistant (R) or susceptible (S). QTL11 is the QTL according to the present invention; its presence is tested with the markers disclosed in the preceding examples. NT means Not tested.
Conclusions: the QTL11 from source D according to the present invention, providing ToBRFV resistance, is not linked to Stemphylium resistance, contrary to the ToBRFV genetic resistance according to WO2020/018783, as the tested plants are not resistant to Stemphylium while comprising the QTL11 of the invention.
It can be concluded that the QTL11 of the invention is thus different from the resistance disclosed in WO2020/018783 on chromosome 11.

### Example 9: Analysis of ToBRFV Resistance Provided by QTL11

A F2 population between Source D and a susceptible parent HMC2 has been obtained. HMC2 is a line highly susceptible to ToBRFV at the leaf level. 134 individual plants have been phenotyped after ToBRFV inoculation for leaf symptoms as described in Example 1.B and genotyped on the basis of SNPs markers of QTL11.

For the trait corresponding to foliar symptoms, the adjusted value of the different tested genotypes regarding the QTL11 locus, was calculated using a mixed model similar to the model used for QTL9.

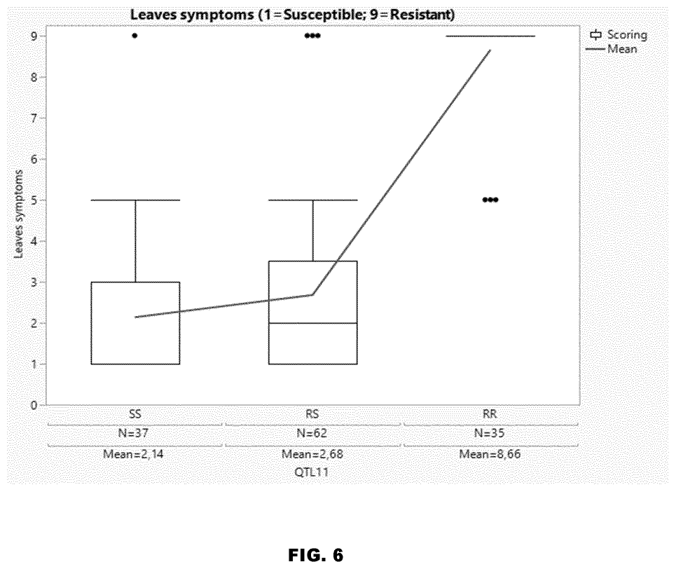

The results are reported in Table M and are illustrated on FIG. 6.

TABLE M

| QTL11 | Mean | Std Dev | Variance | CV | N | Minimum | Maximum | Median |
|---|---|---|---|---|---|---|---|---|
| RR | 8.66 | 1.14 | 1.29 | 13.12 | 35 | 5 | 9 | 9 |
| RS | 2.68 | 2.12 | 4.48 | 79.09 | 62 | 1 | 9 | 2 |
| SS | 2.14 | 1.92 | 3.68 | 89.79 | 37 | 1 | 9 | 1 |

The analysis of the effect of the alleles at QTL11 on the foliar resistance shows a significant recessive effect of the resistant allele, as shown in table M and illustrated in FIG. 6.

1. A Solanum lycopersicum plant resistant to Tomato Brown Rugose Fruit virus (ToBRFV), comprising in its genome a quantitative trait locus (QTL) on chromosome 9 (QTL9) and/or a QTL on chromosome 11 (QTL11) wherein each of the QTLs is introgressed from S. pimpinellifolium and confers to the plant resistance to ToBRFV, and wherein:
the QTL on chromosome 9 is located within the chromosomal region delimited by the SNP TO-0201220 (SEQ ID NO:1) and the SNP having SEQ ID NO:101, and
the QTL on chromosome 11 is located within the chromosomal region delimited by the SNP having SEQ ID NO:102 and the marker having SEQ ID NO:115.

2. The plant according to claim 1, wherein the QTL(s) is (are) present in the genome of the seeds of LVSTBRFVRES2 NCIMB accession number 43591.

3. The plant according to claim 1, wherein the QTL on chromosome 9 can be identified with one of the SNP markers chosen from the list comprising SNP TO-0201220 (SEQ ID NO:1), TO-0201221 (SEQ ID NO:2), TO-0201222 (SEQ ID NO:3), TO-0201223 (SEQ ID NO:4), TO-0201224 (SEQ ID NO:5), TO-0201225 (SEQ ID NO:6), TO-0201226 (SEQ ID NO:7), TO-0201227 (SEQ ID NO:8), TO-0201228 (SEQ ID NO:9), TO-0201229 (SEQ ID NO:10), TO-0201230 (SEQ ID NO:11), TO-0201231 (SEQ ID NO:12), TO-0201232 (SEQ ID NO: 13) and TO-0201233 (SEQ ID NO:14).

4. The plant according to claim 3, wherein the QTL on chromosome 9 can be identified by SNP TO-0201220 and/or SNP TO-0201229.

5. The plant according to claim 1, wherein the QTL on chromosome 9 can be identified by at least one of the following alleles: allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-0201233.

6. The plant according to claim 1, wherein the QTL on chromosome 9 is to be found in the region delimited by TO-0201220 and TO-0201233.

7. The plant according to claim 1, wherein the QTL on chromosome 9 is present homozygously or heterozygously in the genome of the plant.

8. The plant according to claim 1, wherein the QTL on chromosome 11 can be identified with one of the SNPs chosen from the list comprising TO-0201237 (SEQ ID NO:102), TO-0201238 (SEQ ID NO:103), TO-0201239 (SEQ ID NO:104), TO-0201240 (SEQ ID NO:105) and TO-0201241 (SEQ ID NO:106), or with one of the markers chosen from the list comprising SL2.50ch11_9684449 (SEQ ID NO:112), SL2.50ch11_9779896 (SEQ ID NO:113), SL2.50ch11_9823405 (SEQ ID NO:114) and SL2.50ch11_9924232 (SEQ ID NO:115).

9. The plant according to claim 1, wherein the QTL on chromosome 11 can be identified by at least one of the following alleles: allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232.

10. The plant according to claim 1, wherein the QTL on chromosome 11 is to be found in the region delimited by TO-0201237 and SL2.50ch11_9924232.

11. The plant according to claim 1, wherein the QTL on chromosome 11 is present homozygously in the genome of the plant.

12. (canceled)

13. The plant according to claim 1, wherein the plant is a progeny of seeds of LVSTBRFVRES2 (NCIMB accession number 43591).

14. The plant according to claim 1 also containing in its genome sequences the TM-2² gene.

15. The plant according to claim 14, wherein the Tm-2² gene is present heterozygously.

16. The plant according to claim 1, further comprising the Tm-1 gene.

17. A cell of the S. lycopersicum plant according to claim 1, comprising in its genome the QTL9 on chromosome 9 and/or the QTL11 on chromosome 11, independently conferring resistance to ToBRFV.

18. (canceled)

19. A plant part, seeds, explants, reproductive material, scion, cutting, seed, fruit, root, rootstock, pollen, ovule, embryo, protoplast, leaf, anther, stem, petiole or flowers, of a S. lycopersicum plant according to claim 1, wherein the plant part, seeds, explants, reproductive material, scion, cutting, seed, fruit, root, rootstock, pollen, ovule, embryo, protoplast, leaf, anther, stem, petiole or flowers, comprises cells comprising in their genome a QTL9 on chromosome 9 and/or a QTL11 on chromosome 11, wherein each of said QTLs is introgressed from S. pimpinellifolium and confers resistance to ToBRFV, and wherein:
said QTL on chromosome 9 is located within the chromosomal region delimited by the SNP TO-0201220 (SEQ ID NO:1) and the SNP having SEQ ID NO:101, and
said QTL on chromosome 11 is located within the chromosomal region delimited by the SNP having SEQ ID NO:102 and the marker having SEQ ID NO:115.

20. A seed of a S. lycopersicum plant, which develops into the plant according to claim 1.

21. (canceled)

22. (canceled)

23. A method for detecting and/or selecting S. lycopersicum plants comprising a QTL on chromosome 9 and/or a QTL on chromosome 11, introgressed from S. pimpinellifolium and conferring independently ToBRFV resistance, wherein the method comprises the detection of at least one of the following alleles:
allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-0201233, for the QTL on chromosome 9, or
allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 or allele GT of SL2.50ch11_9924232 for the QTL on chromosome 11,
in a genetic material sample of the plant to be selected.

24. (canceled)

25. (canceled)

26. (canceled)

27. A method for conferring resistance to ToBRFV to S. lycopersicum plants, comprising the steps of:
a) Crossing a plant grown from the deposited seeds NCIMB 43591, or progeny thereof, bearing the QTL9 on chromosome 9 and/or the QTL11 on chromosome 11, introgressed from S. pimpinellifolium and conferring independently ToBRFV resistance in NCIMB 43591, and an initial S. lycopersicum plant devoid of the QTL(s),
b) Selecting a plant in the progeny thus obtained, bearing the QTL9 and/or the QTL11;
c) Optionally self-pollinating one or several times the plant obtained at step b) and selecting in the progeny thus obtained a plant having resistance to ToBRFV.

28. (canceled)

29. (canceled)

30. (canceled)
